SEQUENCE LISTING (18 sequences)

SEQ ID NO: 1 - protein, 23 aa, synthetic construct
  source 1..23: note = CD8alpha transmembrane region
YIWAPLAGTCGVLLLSLVITLYC

SEQ ID NO: 2 - protein, 46 aa, synthetic construct
  source 1..46: note = CD8alpha hinge region
TTTPAPRPPTPAPTIASQPLSLRPEACRPAAGGAVHTRGLDFACDI

SEQ ID NO: 3 - protein, 282 aa, synthetic construct
  source 1..282: note = TMIGD2 - Isoform 1
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 4 - protein, 278 aa, synthetic construct
  source 1..278: note = TMIGD2 - Isoform 2
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 5 - protein, 162 aa, synthetic construct
  source 1..162: note = TMIGD2 - Isoform 3
MGSPGMVLGLLVQIWDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 6 - protein, 113 aa, unidentified
  source 1..113: note = CD3zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - DNA, 69 bp, synthetic construct
  source 1..69: mol_type = other DNA; note = CD8alpha transmembrane region
tacatctgggctccactggcaggaacctgtggcgtgctgctgctgtccctggtcatcacactttactgc

SEQ ID NO: 8 - DNA, 138 bp, synthetic construct
  source 1..138: mol_type = other DNA; note = CD8alpha hinge region
accacaactcctgctccaagaccccctacacccgcccctactatcgcttctcagcctctgagtctgagaccagaggcttgcaggcccgcagctggaggagcagtgcacactaggggcctggacttcgcctgcgacatc

SEQ ID NO: 9 - DNA, 849 bp, synthetic construct
  source 1..849: mol_type = other DNA; note = TMIGD2 - Isoform 1
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 10 - DNA, 837 bp, synthetic construct
  source 1..837: mol_type = other DNA; note = TMIGD2 - Isoform 2
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 11 - DNA, 489 bp, synthetic construct
  source 1..489: mol_type = other DNA; note = TMIGD2 - Isoform 3
atggggtccccgggcatggtgctgggcctcctggtgcagatctgggatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 12 - DNA, 339 bp, synthetic construct
  source 1..339: mol_type = other DNA; note = CD3zeta
cgcgtcaagtttagccgatccgcagatgccccagcttaccagcagggacagaaccagctgtataacgagctgaatctgggccggagagaggaatacgacgtgctggacaaaaggcgcgggcgcgaccctgaaatgggaggaaagccacagcgacggaaaaacccccaggagggactgtacaatgaactgcagaaggacaaaatggcagaggcctattcagaaattgggatgaagggagaaagaaggcgcggcaaagggcacgatggcctgtaccaggggctgagcactgccaccaaggacacctatgatgcactgcacatgcaggccctgccccctcgc

SEQ ID NO: 13 - protein, 22 aa, synthetic construct
  source 1..22: note = P2A
GSGATNFSLLKQAGDVEENPGP

SEQ ID NO: 14 - protein, 19 aa, synthetic construct
  source 1..19: note = P2A
ATNFSLLKQAGDVEENPGP

SEQ ID NO: 15 - protein, 335 aa, synthetic construct
  source 1..335: note = EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 16 - DNA, 66 bp, synthetic construct
  source 1..66: mol_type = other DNA; note = P2A
ggaagcggagctactaacttcagcctgctgaagcaggctggagacgtggaggagaaccctggacct

SEQ ID NO: 17 - DNA, 57 bp, synthetic construct
  source 1..57: mol_type = other DNA; note = P2A
gcaacaaacttctctctgctgaaacaagccggagatgtcgaagagaatcctggaccg

SEQ ID NO: 18 - DNA, 1008 bp, synthetic construct
  source 1..1008: mol_type = other DNA; note = EGFRt
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaa

(19) United States
(12) Patent Application Publication
(10) Pub. No.: US 2026/0022157 A1
(43) Pub. Date: Jan. 22, 2026

(54) CHIMERIC ANTIGEN RECEPTORS COMPRISING A TMIGD2 COSTIMULATORY DOMAIN AND ASSOCIATED METHODS OF USING THE SAME

(71) Applicant: Albert Einstein College of Medicine, Bronx, NY (US)

(72) Inventors: Xingxing ZANG, New York, NY (US); Christopher NISHIMURA, Bronx, NY (US)

(21) Appl. No.: 18/994,798

(22) PCT Filed: Jul. 14, 2023
(86) PCT No.: PCT/US2023/070263

§ 371 Date: Jan. 15, 2025

Related U.S. Application Data
(60) Provisional application No. 63/368,549, filed on Jul. 15, 2022.

(51) Int. Cl. C07K 14/705; A61K 35/17; A61K 38/00; A61K 40/11; A61K 40/31; A61K 40/42; A61P 35/00; C07K 14/725; C07K 16/00; C12N 15/62; C12N 15/63

(52) U.S. Cl. CPC C07K 14/70517; A61K 35/17; A61K 38/00; A61K 40/11; A61K 40/31; A61K 40/421; A61P 35/00; C07K 14/7051; C07K 16/00; C12N 15/62; C12N 15/63; C07K 2317/53; C07K 2317/622; C07K 2319/03

(57) ABSTRACT

The present technology provides chimeric antigen receptors (CARs) comprising a TMIGD2 costimulatory domain. In certain embodiments, these CARs comprise an intracellular region comprising the TMIGD2 costimulatory domain and an effector domain, a transmembrane domain, and an extracellular region comprising an antigen binding domain and, optionally a hinge region and/or leader sequence. In other embodiments, these CARs comprise an intracellular region comprising the TMIGD2 costimulatory domain and an effector domain, a transmembrane domain, and an extracellular region comprising an antigen binding domain which specifically binds to tumor associated antigen B7-H3, and, optionally a hinge region and/or leader sequence. Also provided are nucleic acids, vectors, and cells relating to these CARs, as well as methods of using the CARs, nucleic acids, vectors, and cells in the treatment of conditions associated with expression of specific antigens.

### CROSS-REFERENCE TO RELATED APPLICATION AND PRIORITY CLAIM

This application claims priority to U.S. Provisional Application No. 63/368,549 filed on Jul. 15, 2022, the entire contents of which is incorporated herein by reference and relied upon.

### STATEMENT REGARDING FEDERAL FUNDING

This invention was made with government support under CA175495-08 and CA175495-06 awarded by the National Institutes of Health (NIH)/National Cancer Institute (NCI). The government has certain rights in this invention.

### SEQUENCE LISTING INCORPORATED BY REFERENCE

This application contains an ST.26 compliant Sequence Listing, which is submitted concurrently in xml format via Patent Center and is hereby incorporated by reference in its entirety. The .xml copy, created on Jul. 14, 2023, is named 1298078025WO00.xml and is 25 KB in size.

### BACKGROUND

Adoptive transfer of chimeric antigen receptor (CAR) modified T cells or NK cells or macrophages or other immune cells is a potent antigen-specific therapy for treating diseases such as human malignancies. CAR expressing T cells or NK cells or macrophages or other immune cells target tumor-associated antigens expressed on a malignant cell's surface. Since CAR-T or NK cell or macrophages or other immune cell-based therapeutic strategies target an antigen that is already expressed on a cell's surface, issues associated with tumor escape mechanisms involving major histocompatibility complexes can be overcome.

Advances in defining the mechanisms and molecules that regulate immune responses have provided a number of therapeutic targets for treating cancer. For example, costimulatory and coinhibitory molecules play a central role in the regulation of T cell immune responses. However, despite impressive patient responses to antibody agents targeting these costimulatory and coinhibitory molecules, including for example anti-PD-1/PD-L1, checkpoint inhibition therapy still fails in many patients.

There remains a need for new compositions and methods that can improve the effectiveness of these agents, especially those involving CAR modified T cells.

### SUMMARY

In some aspects, the present disclosure provides a chimeric antigen receptor (CAR) comprising: (a) an extracellular region comprising an antigen binding domain; (b) a transmembrane region; and (c) an intracellular region comprising an effector domain and a TMIGD2 costimulatory domain.

In some embodiments, the TMIGD2 costimulatory domain comprises the intracellular region of TMIGD2. In some embodiments, the TMIGD2 costimulatory domain comprises a sequence selected from the group consisting of residues 172-282 of SEQ ID NO:3, 172-278 of SEQ ID NO:4, and residues 52-162 of SEQ ID NO:5. In some embodiments, the TMIGD2 costimulatory domain comprises an amino acid sequence with at least 75% identity to a sequence selected from the group consisting of residues 172-282 of SEQ ID NO:3, 172-278 of SEQ ID NO:4, and residues 52-162 of SEQ ID NO:5.

In some embodiments, the antigen binding domain specifically binds a tumor-associated antigen. In some embodiments, the tumor-associated antigen is selected from the group consisting of HHLA2, CD19; CD20; BCMA; CD22; CD3; CEACAM6; c-Met; EGFR; EGFRvIII; ErbB2; ErbB3; ErbB4; EphA2; IGF1R; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTβR; LIFRβ; LRP5; MUC1; OSMRβ; TCRα; TCRβ; CD25; CD28; CD30; CD33; CD52; CD56; CD79a; CD79b; CD80; CD81; CD86; CD123; CD171; CD276; B7-H3; B7H4; TLR7; TLR9; PTCH1; WT-1; HA1-H; Robo1; α-fetoprotein (AFP); Frizzled; OX40; PRAME; and SSX-2 antigen.

In some embodiments, the antigen binding domain comprises an scFv. In some embodiments, the antigen binding domain comprises a linker. In some embodiments, the linker is a glycine-serine linker. In some embodiments, the glycine-serine linker comprises (GlyxSery)z, wherein x and y are each independently an integer from 0 to 10, provided that x and y are not both 0, and z is an integer from 1 to 10.

In some embodiments, the extracellular region further comprises an N-terminal leader sequence. In some embodiments, the extracellular region further comprises a hinge region. In some embodiments, the hinge region comprises the amino acid sequence set forth in SEQ ID NO:2.

In some embodiments, the transmembrane region comprises a CD8α transmembrane region. In some embodiments, the transmembrane region comprises an amino acid sequence with at least 75% identity to the amino acid sequence set forth in SEQ ID NO:1.

In some embodiments, the effector domain is a CD3ζ effector domain. In some embodiments, the effector domain comprises the amino acid sequence set forth in SEQ ID NO:6. In some embodiments, the effector domain comprises an amino acid sequence with at least 75% identity to the amino acid sequence set forth in SEQ ID NO:5.

In some embodiments, the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:1. In another embodiment, the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:6. In yet another embodiment, the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:1; and (c) the sequence set forth in SEQ ID NO:6. In some embodiments, the CAR further comprises the sequence set forth in SEQ ID NO:2.

In some aspects, the present disclosure provides an isolated polynucleotide, encoding any of the CARs described herein.

In some aspects, the present disclosure provides an expression vector, comprising any of the isolated polynucleotides described herein operably linked to an expression control sequence. In some embodiments, the expression control sequence is a promoter. In some embodiments, the expression control sequence is a promoter.

In some embodiments, the expression vector further comprises an isolated polynucleotide encoding a self-cleaving peptide. In some embodiments, the self-cleaving peptide is a 2A self-cleaving peptide. In some embodiments, the 2A self-cleaving peptide is a P2A peptide. In some embodiments, the isolated polynucleotide encoding the self-cleaving peptide is 3′ of the polynucleotide encoding the CAR. In some embodiments, the isolated polynucleotide encoding the self-cleaving peptide is 5′ of the isolated polynucleotide encoding the marker polypeptide.

In some embodiments, the expression vector further comprises an isolated polynucleotide encoding a transduction marker polypeptide. In some embodiments, the transduction marker polypeptide is a truncated form of epidermal growth factor receptor (EGFRt) or a portion or variant thereof or GFP or a portion or variant thereof.

In some embodiments, the vector is a viral vector.

In some aspects, the present disclosure provides a host cell expressing any of the CARs of the present disclosure, and/or comprising any of the isolated polynucleotides of the present disclosure, and/or comprising the expression vector of the present disclosure.

In some embodiments, the host cell is a T cell, a natural killer (NK) cell, a macrophage, or other immune cell. In some embodiments, the T cell is a CD4+ T cell, a CD8+ T cell, a CD4 CD8 double negative T cell, an NK cell, a macrophage, other immune cell, or any combination thereof. In some embodiments, the T cell is a naïve T cell, a central memory T cell, a stem cell memory T cell, an effector memory T cell, an NK cell, a macrophage, other immune cell, or any combination thereof.

In some embodiments, the host cell further expresses a transduction marker at its cell surface. In some embodiments, the transduction marker is a truncated form of epidermal growth factor receptor (EGFRt) or a portion or variant thereof or GFP or a portion or variant thereof.

In some aspects, the present disclosure provides a method of treating a disease or condition in a subject in need thereof comprising administering to the subject an effective amount of the host cell of the present disclosure.

In some embodiments, the disease or condition is a malignancy. In some embodiments, the malignancy is a cancer. In some embodiments, the cancer is selected from the group consisting of prostate cancer, liver cancer, melanoma, leukemia, lymphoma, breast cancer, ovarian cancer, pancreatic cancer, colorectal cancer, lung cancer, bladder cancer, renal cancer, brain cancer, stomach, thyroid, anus, small intestine, bone, cervix, endometrium, esophagus, eye, gallbladder, thymus, sarcoma and osteosarcoma. In some embodiments, the cancer comprises a solid tumor. In some embodiments, the cancer comprises a hematologic malignancy.

In some aspects, the present disclosure provides a method of eliciting an immune response against a tumor-associated antigen that specifically binds a CAR of the present disclosure, the method comprising administering to a subject in need thereof an effective amount of any of the host cells of the present disclosure.

In some aspects, the present disclosure provides a composition comprising a CAR of the present disclosure and pharmaceutically acceptable excipient, carrier, or diluent.

In some aspects, the present disclosure provides a composition comprising a cell of the present disclosure and a pharmaceutically acceptable excipient, carrier, or diluent.

These and other embodiments of the present disclosure will be disclosed in further detail herein below.

### BRIEF DESCRIPTION OF THE DRAWINGS

FIG. 1 is a schematic representation of a CAR vector in accordance with an embodiment of the present disclosure. The vector encodes a CAR comprising an IgG1 Vk leader sequence, VL and VH domains with an intervening linker sequence, a CD8a hinge and transmembrane region, a TMIGD2 intracellular tail costimulatory domain, and a CD3ζ effector domain. The vector further comprises a self-cleaving peptide P2A, the GMCSFR leader and the polypeptide marker EGFRt.

FIG. 2 depicts a representative graph showing that T cells expressing a CAR in accordance with an embodiment of the present disclosure kill Raji human tumor cells.

FIG. 3 shows the in vivo therapeutic efficacy of CD19-TMIGD2 CAR-T cells in a Raji lymphoma model. Statistics was calculated using the log-rank (Mantel-Cox) test. N=3, p=0.0295.

FIG. 4A is a schematic representation depicting example B7-H3 and CD19 CAR construct designs.

FIG. 4B depicts a representative graph showing a single-killing co-culture assay of B7-H3.28.ζ, B7-H3.BB.ζ, B7-H3.TMI.ζ, B7-H3.28.BB.ζ, B7-H3.TMI.ζ, CD19.28.BB.ζ CAR-T cells and U118-Luc tumor cells. CAR transduction efficiency was adjusted to 50%.

FIG. 4C depicts a representative graph showing a single-killing co-culture assay of B7-H3.28.ζ, B7-H3.BB.ζ, B7-H3.TMI.ζ, B7-H3.28.BB.ζ, B7-H3.TMI.ζ, CD19.28.BB.ζ CAR-T cells and HCC827-Luc tumor cells. CAR transduction efficiency was adjusted to 50%.

FIG. 4D depicts a representative graph showing a single-killing co-culture assay of B7-H3.28.ζ, B7-H3.BB.ζ, B7-H3.TMI.ζ, B7-H3.28.BB.ζ, B7-H3.TMI.BB.ζ, CD19.28.BB.ζ CAR-T cells and THP-1 tumor cells. CAR transduction efficiency was adjusted to 50%.

FIG. 4E depicts a representative graph showing a tumor growth curve from time lapse co-culture assay of B7-H3.TMI.ζ, B7-H3.28.BB.ζ, CD19.28.BB.ζ CAR-T cells and U118-Luc tumor cells where the tumor burden was quantified based on area of signal (tdTomato) over time.

FIG. 4F depicts a representative graph showing a tumor growth curve from time lapse co-culture assay of B7-H3.TMI.ζ, B7-H3.28.BB.ζ, CD19.28.BB.ζ CAR-T cells and HCC827-Luc tumor cells where the tumor burden was quantified based on area of signal (tdTomato) over time.

FIG. 4G depicts representative graphs showing B7-H3.TMI.ζ, B7-H3.28.BB.ζ, CD19.28.BB.ζ CAR-T cytokine release after 24-hour co-culture with HCC827 tumor cells.

FIG. 4H shows a gating strategy used to detect B7-H3 and CD19 CAR-T cell and memory subsets.

FIG. 4I depicts representative flow cytometry plots and bar graph showing B7-H3.28.ζ, B7-H3.BB.ζ, B7-H3.TMI.ζ, B7-H3.28.BB.ζ, B7-3.TMI.BB.ζ, CD19.28.BB.ζ CAR-T cell transduction efficiency.

FIG. 4J depicts representative graphs showing memory populations of B7-H3.28.ζ, B7-H3.BB.ζ, B7-H3.TMI.ζ, B7-H3.28.BB.ζ, B7-H3.BB.ζ, CD19.28.TMI.BB.ζ CAR-T cell constructions.

FIG. 4K depicts representative graphs showing summary data regarding expansion of B7-H3.28.ζ, B7-H3.BB.ζ, B7-H3.TMI.ζ, B7-H3.28.BB.ζ, B7-H3.TMI.BB.ζ, CD19.28.BB.ζ CAR-T cell constructs in the absence of tumor antigen.

FIG. 5A is a schematic representation of a representative experimental design timeline for in vivo HCC827-Luc cell killing assays using TMIGD2 CAR-T cells.

FIG. 5B shows representative bioluminescent images of mice at representative survival timepoints following administration of B7-H3.TMI.ζ, B7-H3.28.BB.ζ, CD19.28.BB.ζ CAR-T cells targeting engrafted HCC827-Luc cells according to the experimental design illustrated, in part, in FIG. 5A.

FIG. 5C depicts representative graphs showing the quantification of tumor signal from individual mice and summary data comparing experimental groups at representative survival timepoints following administration of B7-H3.TMI.ζ, B7-H3.28.BB.ζ, CD19.28.BB.ζ CAR-T cells targeting engrafted HCC827-Luc cells according to the experimental design illustrated, in part, in FIG. 5A.

FIG. 5D depicts representative graphs showing the Kaplan-Meier survival curve of the experimental groups of B7-H3.TMI.ζ, B7-H3.28.BB.ζ, CD19.28.BB.ζ CAR-T cells from FIG. 5C.

FIG. 5E is a schematic representation of a representative experimental design timeline for in vivo U118-Luc cell killing assays using TMIGD2 CAR-T cells.

FIG. 5F shows representative bioluminescent images of mice at representative survival timepoints following administration of B7-H3.TMI.ζ, B7-H3.28.BB.ζ, CD19.28.BB.ζ CAR-T cells targeting engrafted U118-Luc cells according to the experimental design illustrated, in part, in FIG. 5E.

FIG. 5G depicts representative graphs showing the quantification of tumor signal from individual mice and summary data comparing experimental groups at representative survival timepoints following administration of B7-H3.TMI.ζ, B7-H3.28.BB.ζ, CD19.28.BB.ζ CAR-T cells targeting engrafted U118-Luc cells according to the experimental design illustrated, in part, in FIG. 5E.

FIG. 5H depicts representative graphs showing the Kaplan-Meier survival curve of the experimental groups of B7-H3.TMI.ζ, B7-H3.28.BB.ζ, CD19.28.BB.ζ CAR-T cells from FIG. 5F.

FIG. 5I is a schematic representation of a representative experimental design timeline for in vivo PANC-1-Luc cell killing assays using TMIGD2 CAR-T cells.

FIG. 5J shows representative bioluminescent images of mice at representative survival timepoints following administration of B7-H3.TMI.ζ, B7-H3.28.BB.ζ, CD19.28.BB.ζ CAR-T cells targeting engrafted PANC-1-Luc cells according to the experimental design illustrated, in part, in FIG. 5I.

FIG. 5K depicts representative graphs showing the quantification of tumor signal from individual mice and summary data comparing experimental groups at representative survival timepoints following administration of B7-H3.TMI.ζ, B7-H3.28.BB.ζ, CD19.28.BB.ζ CAR-T cells targeting engrafted PANC-1-Luc according to the experimental design illustrated, in part, in FIG. 5I.

FIG. 5L depicts representative graphs showing the Kaplan-Meier survival curve of the experimental groups of B7-H3.TMI.ζ, B7-H3.28.BB.ζ, CD19.28.BB.ζ CAR-T cells from FIG. 5I.

FIG. 6A is a schematic representation depicting example B7-H3.TMI.ζ, B7-H3.28.BB.ζ, CD19.28.BB.ζ CAR-Luc construct designs.

FIG. 6B is a schematic representation of a representative experimental design timeline for in vivo HCC827-Luc cell killing assays using TMIGD2 CAR-T cells.

FIG. 6C shows representative bioluminescent images of mice at representative survival timepoints following administration of B7-H3.TMI.ζ, B7-H3.28.BB.ζ, CD19.28.BB.ζ CAR-T cells targeting engrafted UCC827-Luc cells according to the experimental design illustrated, in part, in FIG. 6B.

FIG. 6D depicts representative graphs showing line graphs depicting B7-H3.TMI.ζ, B7-H3.28.BB.ζ, CD19.28.BB.ζ CAR-T luciferase signals from individual mice according to the experimental design illustrated, in part, in FIG. 6B.

FIG. 6E depicts representative graphs showing a bar graph comparing B7-H3.TMI.ζ, B7-H3.28.BB.ζ, CD19.28.BB.ζ CAR-T luciferase signals according to the experimental design illustrated, in part, in FIG. 6B.

FIG. 6F depicts representative graphs showing peak bioluminescent signal from each mouse for B7-H3.TMI.ζ, B7-H3.28.BB.ζ, CD19.28.BB.ζ CAR-T cells over the course of the experiment.

FIG. 6G depicts representative graphs showing the quantification of B7-H3.TMI.ζ, B7-H3.28.BB.ζ, CD19.28.BB.ζ CAR-T cells in lung tissue in the experiment.

FIG. 6H depicts representative graphs showing quantification of B7-H3.TMI.ζ, B7-H3.28.BB.ζ, CD19.28.BB.ζ CAR-T cells in spleen tissue in the experiment.

FIG. 6I depicts representative graphs showing quantification of B7-H3.TMI.ζ, B7-H3.28.BB.ζ, CD19.28.BB.ζ CAR-T cells in blood in the experiment.

FIG. 7A shows a heatmap depicting all differentially expressed genes (DEGs) (p adj<0.05; fold change >0.5) from B7-H3.TMI.ζ CAR-T cells and B7-H3.28.BB.ζ CAR-T cells vs CD19.28.BB.ζ CAR-T cells as shown in FIG. 4A.

FIG. 7B depicts a representative graph showing a Venn diagram containing unique and shared DEGs between B7-H3.TMI.ζ CAR-T cells B7-H3.28.BB.ζ CAR-T cells.

FIG. 7C depicts representative graphs showing gene set enrichment analysis (GSEA) pathway analysis comparing B7-H3.TMI.ζ and B7-H3.28.BB.ζ CAR-T cells vs CD19.28.BB.ζ CAR-T cells obtained from Reactome and Hallmark data sets.

FIG. 7D depicts representative graphs showing a comparison of percent ATP generated from glycolysis and mitochondria in B7-H3.TMI.ζ vs B7-H3.28.BB.ζ CAR-T cells cultured without tumor cells (baseline) or with HCC827 tumor cells (coculture) for 24 hours.

FIG. 7E depicts representative graphs showing a comparison of percent ATP generated from glycolysis and mitochondria in B7-H3.TMI.ζ vs B7-H3.28.BB.ζ CAR-T cells cultured without tumor cells (baseline) or with HCC827 tumor cells (coculture) for 7 days with repeat additions of tumor cells given every two days.

FIG. 7F shows a heatmap depicting DEGs from B7-H3.TMI.ζ CAR-T cells compared to B7-H3.28.BB.ζ CAR-T cells.

FIG. 7G depicts representative graphs showing GSEA pathway analysis of RNA sequencing data obtained from Reactome and Hallmark data sets for B7-H3.TMI.ζ vs B7-H3.28.BB.ζ CAR-T cells.

FIG. 7H depicts representative graphs showing dot plot containing overrepresentation analysis of highly expressed DEGs (fold change >±1.5) for B7-H3. TMI.ζ vs B7-H3.28.BB.ζ CAR-T cells.

FIG. 7I shows a heatmap of DEGs from curated gene set describing glycolysis and oxidative phosphorylation proteins for B7-H3.TMI.ζ vs B7-H3.28.BB.ζ CAR-T cells.

FIG. 8A shows a heatmap depicting DEGs from in vivo lung-infiltrating T cells in mice injected with B7-H3.TMI.ζ CAR-T cells compared to B7-H3.28.BB.ζ CAR-T cells.

FIG. 8B depicts representative graphs showing GSEA pathway analysis of DEGs.

FIG. 8C depicts representative graphs showing overrepresentation analysis of highly expressed DEGs (fold change >±1.5) for B7-H3.TMI.ζ vs B7-H3.28.BB.ζ CAR-T cells.

FIG. 8D shows a heatmap depicting average gene expression of DEGs found in metabolism gene signature for B7-H3.TMI.ζ vs B7-H3.28.BB.ζ CAR-T cells.

FIG. 8E shows a heatmap depicting average gene expression of DEGs found in T cell dysfunction gene signature for B7-H3.TMI.ζ vs B7-H3.28.BB.ζ CAR-T cells.

FIG. 8F shows a heatmap depicting average gene expression of DEGs found in inhibitory protein gene signature for B7-H3.TMI.ζ vs B7-H3.28.BB.ζ CAR-T cells.

FIG. 8G depicts representative graphs showing a Venn diagram containing overlapping significant genes (p adj<0.05) in either B7-H3.TMI.ζ or B7-H3.28.BB.ζ CAR-T cells from in vivo and in vitro RNA sequencing experiments directly comparing the two constructs.

FIG. 8H depicts representative graphs showing ORA analysis of shared genes in FIG. 8G for B7-H3.TMI.ζ vs B7-H3.28.BB.ζ CAR-T cells.

FIG. 9A shows tSNE plots from CAR+ populations concerning phenotypic analysis comparing B7-H3.TMI.ζ and B7-H3.28.BB.ζ CAR-T cells after CAE with HCC827 tumor cells.

FIG. 9B shows CAR transduction efficiency during chronic antigen exposure (CAE) concerning phenotypic analysis comparing B7-H3.TMI.ζ and B7-H3.28.BB.ζ CAR-T cells after CAE with HCC827 tumor cells.

FIG. 9C depicts representative graphs showing cell surface markers used phenotype exhaustion, memory, activation, and CD4/CD8 CAR-T cell populations during CAE.

FIG. 9D depicts representative graphs showing the luciferase signal from non-transduced, B7-H3.TMI.ζ, B7-H3.28.BB.ζ, or CD19-28.BB.ζ CAR-transduced Jurkat (NFAT responsive luciferase reporter) cells after culture in the indicated conditions.

FIG. 9E depicts representative graphs showing a phenotypic analysis of CAR+CD4+ T cells during CAE.

FIG. 9F depicts representative graphs showing a phenotypic analysis of CAR+CD8+ T cells during CAE.

### DETAILED DESCRIPTION

The present disclosure provides CARs comprising a TMIGD2 costimulatory domain, as well as nucleic acid molecules and vectors encoding these CARs, cells expressing these CARs, and methods of using said CARs, nucleic acid molecules, vectors, and cells.

HHLA2 (HERV-H LTR Associating 2) is a functional member of the B7 family and TMIGD2 (NCBI accession number NP_653216.2) has previously been identified as a costimulatory receptor for HHLA2 (Zhao 2013; Janakiram 2015a; Janakiram 2015b). There are at least three isoforms of TMIGD2: isoform 1 (SEQ ID NO:3, NCBI NP_653216.2), isoform 2 (SEQ ID NO:4, NCBI NP_001162597.1), and isoform 3 (SEQ ID NO: 5, NCBI NP_001295161.1). Exemplary DNA sequences encoding isoforms 1-3 are set forth in SEQ ID NO:9 (NCBI NM_144615.), SEQ ID NO:10 (NCBI NM_001169126.1), and SEQ ID NO:11 (NCBI NM_001308232), respectively.

While most CARs incorporate CD28 or 41-BB as costimulatory molecules, the present disclosure relates to the CARs using TMIGD2 as a costimulatory molecule. TMIGD2 is expressed on the majority of naïve T cells in humans. After antigenic stimulation almost all T cells lose TMIGD2 expression, and this loss is associated with increased levels of PD-1, exhaustion, and senescence (Zhu 2013; Crespo 2017; Janakiram 2017). The use of TMIGD2 to provide costimulation in a CAR-T cell therapy presents a unique opportunity to maintain TMIGD2 costimulatory signaling and preserve functionally active CAR-T cells.

Exemplary CARs of the present disclosure comprise (a) an extracellular region comprising a binding domain (e.g., an scFv); (b) a transmembrane region; and (c) an intracellular region comprising an effector domain and a TMIGD2 costimulatory domain. The TMIGD2 costimulatory domain may comprise all or a portion of the intracellular region of any TMIGD2 isoform, including any of isoforms 1-3, or a variant thereof.

The CARs of the present disclosure are useful in cellular immunotherapies (e.g., T cell and/or natural killer (NK) cell) for treating a disease associated with expression of one or more antigens, such as cancers. In some embodiments, when administered to a subject having malignant cells that express one or more antigens associated with cancer, CARs of the present disclosure reduce and/or suppress growth, area, volume, and/or spread of the malignant cells, eliminate (e.g., kill) malignant cells, and/or increase survival of the subject to a greater degree and/or for a longer period of time than cells that do not comprise a CAR of the present disclosure.

The following description of the present disclosure is merely intended to illustrate various embodiments of the present disclosure. As such, the specific modifications discussed herein are not to be construed as limitations on the scope of the present disclosure. It will be apparent to one skilled in the art that various equivalents, changes, and modifications may be made without departing from the scope of the present disclosure, and it is understood that such equivalent embodiments are to be included herein.

Reference throughout this specification to “one example,” “an example,” “one embodiment,” “an embodiment,” “one aspect,” or “an aspect” means that a particular feature, structure, or characteristic described in connection with the example is included in at least one example of the present disclosure. Thus, the occurrences of the phrases “in one example,” “in an example,” “one embodiment,” “an embodiment,” “one aspect,” or “an aspect” in various places throughout this specification are not necessarily all referring to the same example, embodiment, and/or aspect.

The headings provided herein are for convenience only and are not intended to limit or interpret the scope or meaning of the present disclosure.

### Definitions

Any concentration range, percentage range, ratio range, or integer range referenced herein is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense that one having skill in the art would understand the convention (e.g., “a system having at least one of A, B, or C” would include but not be limited to systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting.

A “nucleic acid molecule” or “polynucleotide” refers to a polymeric compound including covalently linked nucleotides comprising natural subunits (e.g., purine or pyrimidine bases). Purine bases include adenine, and guanine, and pyrimidine bases including uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

“Percent identity” or “percent sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

A “conservative substitution” refers to an amino acid substitution that does not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. Here, “plasmid,” “expression plasmid,” “virus” and “vector” are often used interchangeably.

The term “introduced” in the context of inserting a nucleic acid molecule into a cell, means “transfection,” or “transformation” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, CARs or enzymes, or other nucleic acid molecule additions, deletions, substitutions or other functional disruption of a cell's genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4+ cells while depleting CD8+ cells, or enriching for CD8+ cells while depleting CD4+ cells, or combinations thereof.

“Chimeric antigen receptor” (CAR) refers to a CAR of the present disclosure engineered to contain two or more naturally occurring (or engineered) amino acid sequences linked together in a way that does not occur naturally or does not occur naturally in a particular cell, which CAR can function as a receptor when present on a surface of a cell. CARs of the present disclosure include an extracellular portion comprising an antigen-binding domain, such as one obtained or derived from an immunoglobulin, such as an scFv derived from an antibody linked to a transmembrane region and one or more intracellular signaling domains (optionally containing costimulatory domain(s)) (see, e.g., Sadelain et al., 2013; see also Harris & Kranz, 2016; Stone et al., 2014).

The term “variable region” or “variable domain” refers to an antibody heavy or light chain, that is involved in binding to antigen. Variable domains of antibody heavy (V_{H}) and light (V_{L}) chains each generally comprise four generally conserved framework regions (FRs) and three CDRs. Framework regions separate CDRs and CDRs are situated between framework regions.

The terms “complementarity determining region,” and “CDR,” are synonymous with “hypervariable region” or “HVR,” and are known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” as used herein refers to an immunogenic molecule that provokes an immune response. This immune response may involve antibody production, activation of specific immunologically-competent cells, or both. An antigen may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

A “binding domain” (also referred to as a “binding region”), as used herein, refers to a molecule or portion thereof that possesses the ability to specifically and non-covalently associate, unite, or combine with a target, such as an scFv. A binding domain includes any naturally occurring, synthetic, semi-synthetic, or recombinantly produced binding partner for a biological molecule, a molecular complex, or other target of interest. Exemplary binding domains include single chain immunoglobulin variable regions, receptor ectodomains, ligands, or synthetic polypeptides selected for their specific ability to bind to a biological molecule, a molecular complex or other target of interest.

As used herein, an “effector domain” is an intracellular portion or domain of a CAR or receptor that can directly or indirectly promote a biological or physiological response in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

A “transmembrane region,” as used herein, is a portion of a transmembrane protein that can insert into or span a cell membrane.

“Treat” or “treatment” or “ameliorate” refers to medical management of a disease, disorder, or condition of a subject. In general, an appropriate dose or treatment regimen comprising a cell expressing a CAR of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof.

As used herein, “hyperproliferative disorder” and “proliferative disorder” refer to excessive growth or proliferation as compared to a normal or undiseased cell. Exemplary hyperproliferative disorders and proliferative disorders include tumors, cancers, neoplastic tissue, carcinoma, sarcoma, malignant cells, pre malignant cells.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

A “therapeutically effective amount” or “effective amount” of a cell expressing a CAR of this disclosure, refers to an amount of CAR expressing cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

The term “pharmaceutically acceptable” with regard to a carrier, excipient, or diluent means that the carrier, excipient, or diluent is suitable for administration to a human or other non-human mammalian subject and generally recognized as safe or not causing a serious adverse event.

The term “adoptive immune therapy” or “adoptive immunotherapy” as used herein refers to administration of naturally occurring or genetically engineered, disease-antigen-specific immune cells, such as T cells. Adoptive cellular immunotherapy may be autologous (immune cells are from the recipient), allogeneic (immune cells are from a donor of the same species) or syngeneic (immune cells are from a donor genetically identical to the recipient).

A “T cell” or “T lymphocyte” is an immune system cell that matures in the thymus and produces T cell receptors (TCRs), including αβT cells and γδT cells. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or T_{CM}).

A “natural killer cell” or “NK cell” as used herein refers to a cell that is activated in response to interferons or macrophage-derived cytokines, contains viral infections while the adaptive immune response is generating antigen-specific cytotoxic T cells that can clear the infection, and expresses CD56.

In addition, it should be understood that the individual constructs, or groups of constructs, derived from the various combinations of the structures and subunits described herein, are disclosed by the present disclosure to the same extent as if each construct or group of constructs was set forth individually. Thus, selection of particular structures or particular subunits is within the scope of the present disclosure.

The terminology used in the description is intended to be interpreted in its broadest reasonable manner, even though it is being used in conjunction with a detailed description of identified embodiments.

### Chimeric Antigen Receptors (CARs)

Provided herein are CARs comprising a TMIGD2 costimulatory domain, as well as compositions comprising these CARs. In certain embodiments, the CARs provided herein comprise (a) an extracellular region comprising a binding domain that specifically binds to a tumor-associated antigen expressed on the surface of a cell, such as a malignant cell, (b) a transmembrane region, and (c) an intracellular region comprising an effector domain and a TMIGD2 costimulatory domain. Also provided herein are cells expressing these CARs and compositions thereof, and unit doses of these cells and compositions. In some embodiments, these unit doses comprise (i) a composition comprising at least about 50% modified CD4+ T cells, combined with (ii) a composition comprising at least about 50% modified CD8+ T cells, in about a 1:1 ratio, wherein the unit dose contains a reduced amount or substantially no naïve T cells.

### TMIGD2 Costimulatory Domain

A costimulatory domain refers to a portion of an intracellular domain that includes a costimulatory molecule. Costimulatory molecules are cell surface molecules required for efficient response of lymphocytes to antigens, rather than antigen receptors or their ligands.

The CARs provided herein contain a TMIGD2 costimulatory domain comprising all or a portion of the intracellular region of TMIGD2 or a variant thereof. In certain embodiments, the TMIGD2 intracellular region is derived from TMIGD2 isoform 1 (SEQ ID NO:3), isoform 2 (SEQ ID NO:4), or isoform 3 (SEQ ID NO:5). In certain embodiments wherein the TMIGD2 costimulatory domain comprises a portion of the intracellular region of TMIGD2, that portion is sufficient to transduce a signal normally associated with TMIGD2 binding to HHLA2.

In certain embodiments, the TMIGD2 intracellular region is derived from isoform 1 or 3 and comprises, consists of, or consists essentially of the amino acid sequence set forth at residues 172-282 of SEQ ID NO:3 or residues 52-162 of SEQ ID NO: 5 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or residues 52-162 of SEQ ID NO:5 or a portion thereof.

In certain embodiments, the TMIGD2 intracellular region is derived from isoform 2 and comprises, consists of, or consists essentially of the amino acid sequence set forth at residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to amino acids 172-278 of SEQ ID NO:4 or a portion thereof.

In certain embodiments wherein the TMIGD2 costimulatory domain comprises an amino acid sequence with less than 100% identity to a TMIGD2 intracellular region or a portion thereof, e.g., a TMIGD2 intracellular region of isoforms 1-3, all of the substitutions giving rise to the sequence differences are conservative substitutions. In other embodiments, the TMIGD2 costimulatory domain may comprise one or more non-conservative substitutions versus the TMIGD2 intracellular region. In certain embodiments, the TMIGD2 costimulatory domain comprises one, two, three, four, or five or more conservative substitutions versus a TMIGD2 intracellular region.

### Intracellular Effector Domain

In certain embodiments, the CARs provided herein comprise an intracellular effector domain. In some embodiments, the intracellular effector domain is a CD3ζ effector domain or a functional portion or variant thereof.

In some embodiments, the CD3ζ effector domain comprises, consists of, or consists essentially of the amino acid sequence of SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO:6.

In certain embodiments wherein the CARs provided herein comprise an intracellular effector domain, the effector domain is directly adjacent to the TMIGD2 costimulatory domain. In other embodiments, the effector domain and TMIGD2 costimulatory domain are separated by one or more amino acids.

### Transmembrane Region

In certain embodiments, the CARs provided herein comprise a transmembrane region connecting the extracellular and intracellular regions.

In certain embodiments, the transmembrane region is derived from CD8α. In certain of these embodiments, the transmembrane region comprises, consists of, or consists essentially of the amino acid sequence of SEQ ID NO:1 or a portion thereof, or a sequence with at least 75%, 80%, 85%, 90%, 91%, 92%, 93%, 94%, 95%, 96%, 97%, 98%, 99% identity to SEQ ID NO:1.

### Extracellular Region

In certain embodiments, the CARs provided herein comprise an extracellular region that includes a target specific-binding element (also known as an antigen binding domain).

In certain embodiments, the antigen binding domains in the extracellular region are derived from antibodies and comprise antigen-binding portions thereof. For example, in certain embodiments the antigen binding domains comprise all or a portion of an antibody V_{H} chain (e.g., a V_{H} variable domain or one or more CDRs thereof), all or a portion of an antibody V_{L} chain (e.g., a V_{L} variable domain or one or more CDRs thereof), or both. In certain of these embodiments, the antigen binding domain is an scFv. In certain of these embodiments, the scFv linker domain comprises a peptide linker between the V_{L} and V_{H} components. For example, the scFvs may be designed so that the C-terminal end of the V_{L} domain is linked to the N-terminal end of the V_{H} domain by the peptide linker ((N)V_{L}(C)-linker-(N)V_{H}(C)), or such that the C-terminal end of the V_{H} domain is linked to the N-terminal end of the V_{L} domain by the peptide linker (N)V_{H}(C)-linker-(N)V_{L}(C). Exemplary linkers include those having a glycine-serine amino acid chain comprising one to ten repeats of GlyₓSer_{y}, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser).

Anti-B7-H3 CAR-T binding domain sequences useful in the present technology are described, for example, in PCT/US2020/66002 entitled “Chimeric Antigen Receptors Targeting B7-H3 (CD276) and Associated Methods, and PCT/US2019/61887 entitled “Monoclonal antibodies against IgV domain of B7-H3 and uses thereof,” the techniques and sequences of which are herein incorporated by reference in their entireties.

In some embodiments, the binding domain specifically binds to one or more tumor-associated antigens selected from a: HHLA2; CD19; CD20; BCMA; CD22; CD3; CEACAM6; c-Met; EGFR; EGFRvIII; ErbB2; ErbB3; ErbB4; EphA2; IGF1R; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTβR; LIFRβ; LRP5; MUC1; OSMRβ; TCRα; TCRβ; CD25; CD28; CD30; CD33; CD52; CD56; CD79a; CD79b; CD80; CD81; CD86; CD123; CD171; CD276; B7-H3; B7H4; TLR7; TLR9; PTCH1; WT-1; HA1-H; Robo1; α-fetoprotein (AFP); Frizzled; OX40; PRAME; and/or SSX-2 antigen.

Sources of binding domains are known in the art, including known antibodies, methods of generating antibodies, and binding domains described herein. In some embodiments, the tumor associated antigen is CD19. In particular embodiments, the binding domain is derived from an anti-CD19 antibody, such as, for example, FMC-63 antibody, MOR208, blinatumomab, MEDI-551, Meck patent anti-CD19 antibody, Xmab5871, or MDX-1342.

In some embodiments, the antigen-specific receptor binding domain is derived from FMC-63 antibody, MOR208, blinatumomab, MEDI-551, Merck patent anti-CD19 antibody, Xmab5871, or MDX-1342 has a VH, or (i.e., and/or) a VL having at least about 80%, 85%, 90%, 95%, 96%, 96%, 98%, 99%, or more amino acid sequence identity to that of the antibody variable regions or scFv thereof from FMC-63 antibody, MOR208, blinatumomab, MEDI-551, Merck patent anti-CD19 antibody, Xmab5871, or MDX-1342, or has CDRs or functional CDR variants according to any one of these antibodies.

In some embodiments, the antigen binding domain is directly adjacent to the transmembrane domain. In other embodiments, there are one or more intervening residues linking the antigen binding domain and the transmembrane domain, for example a hinge region. In certain embodiments wherein the CARs comprise a hinge region, the hinge region is derived from CD8α, and in certain of these embodiments the hinge region comprises, consists of, or consists essentially of the amino acid sequence of SEQ ID NO: 2 or a portion thereof, or a sequence with at least 75%, 80%, 85%, 90%, 91%, 92%, 93%, 94%, 95%, 96%, 97%, 98%, 99% identity to SEQ ID NO:2.

In some embodiments, the extracellular region further comprises a leader sequence, which includes but is not limited to a leader peptide. For example, the leader peptide can be an IgG1 V_{K} leader domain, or a portion or variant thereof bound to the N-terminal end of the V_{H} domain or the V_{L} domain of the sFv.

### SPECIFIC EMBODIMENTS

In certain embodiments, the CARs provided herein comprise (a) a TMIGD2 costimulatory domain and (b) an intracellular CD3ζ effector domain or a portion or variant thereof. In certain of these embodiments (a) the TMIGD2 costimulatory domain comprises, consists of, or consists essentially of the amino acid sequence set forth at residues 172-282 of SEQ ID NO:3 or a portion thereof, residues 52-162 of SEQ ID NO:5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; and (b) the CD3ζ effector domain comprises, consists of, or consists essentially of the amino acid sequence set forth in SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO:6 or a portion thereof. In certain of these embodiments, the TMIGD2 costimulatory domain and the intracellular CD3ζ effector domain are directly adjacent to one another. In other embodiments, there are one or more amino acids separating the TMIGD2 costimulatory domain and the intracellular CD3ζ effector domain. In certain embodiments, the CARs provided herein comprise the amino acid sequence set forth at residues 172-282 of SEQ ID NO:3 or a portion thereof and the amino acid sequence set forth in SEQ ID NO:6. In certain embodiments, the CARs provided herein comprise the amino acid sequence set forth at residues 52-162 of SEQ ID NO:5 or a portion thereof and the amino acid sequence set forth in SEQ ID NO:6. In certain embodiments, the CARs provided herein comprise the amino acid sequence set forth at residues 172-278 of SEQ ID NO:4 or a portion thereof and the amino acid sequence set forth in SEQ ID NO:6.

In certain embodiments, the CARs provided herein comprise (a) a TMIGD2 costimulatory domain and (b) a CD8α transmembrane region or a portion or variant thereof. In certain of these embodiments (a) the TMIGD2 costimulatory domain comprises, consists of, or consists essentially of the amino acid sequence set forth at residues 172-282 of SEQ ID NO:3 or a portion thereof, residues 52-162 of SEQ ID NO:5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; and (b) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to the sequence set forth in SEQ ID NO:1 or a portion thereof. In certain embodiments, the CD8α transmembrane region is directly adjacent to the TMIGD2 costimulatory domain, while in other embodiments there are one or more amino acids separating the CD8α transmembrane region and the TMIGD2 costimulatory domain. In certain embodiments, the CARs provided herein comprise the amino acid sequence set forth at residues 172-282 of SEQ ID NO:3 or a portion thereof and the amino acid sequence set forth in SEQ ID NO:1. In certain embodiments, the CARs provided herein comprise the amino acid sequence set forth at residues 52-162 of SEQ ID NO: 5 or a portion thereof and the amino acid sequence set forth in SEQ ID NO:1. In certain embodiments, the CARs provided herein comprise the amino acid sequence set forth at residues 172-278 of SEQ ID NO:4 or a portion thereof and the amino acid sequence set forth in SEQ ID NO:1.

In certain embodiments, the CARs provided herein comprise (a) a TMIGD2 costimulatory domain, (b) an intracellular CD3ζ effector domain or a portion or variant thereof, and (c) a CD8α transmembrane region or a portion or variant thereof. In certain of these embodiments (a) the TMIGD2 costimulatory domain comprises, consists of, or consists essentially of the amino acid sequence set forth at residues 172-282 of SEQ ID NO: 3 or a portion thereof, residues 52-162 of SEQ ID NO:5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; (b) the CD3ζ effector domain comprises, consists of, or consists essentially of the amino acid sequence set forth in SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO: 6 or a portion thereof; and (c) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof. In certain embodiments, the TMIGD2 costimulatory domain is located between the CD8α transmembrane region and the CD3ζ effector domain. In other embodiments, the CD3ζ effector domain is located between the CD8α transmembrane region and the TMIGD2 costimulatory domain. In certain embodiments, the CD8α transmembrane region is directly adjacent to the CD3ζ effector domain or TMIGD2 costimulatory domain, while in other embodiments there are one or more amino acids separating the CD8α transmembrane region and the CD3ζ effector domain or TMIGD2 costimulatory domain. Similarly, in certain embodiments the TMIGD2 costimulatory domain and CD3ζ effector domain are directly adjacent to one another, while in other embodiments there are one or more amino acids separating the TMIGD2 costimulatory domain and the intracellular CD3ζ effector domain. In certain embodiments, the CARs provided herein comprise the amino acid sequence set forth at residues 172-282 of SEQ ID NO:3 or a portion thereof, the amino acid sequence set forth in SEQ ID NO:6, and the amino acid sequence set forth in SEQ ID NO:1. In certain embodiments, the CARs provided herein comprise the amino acid sequence set forth at residues 52-162 of SEQ ID NO:5 or a portion thereof, the amino acid sequence set forth in SEQ ID NO:6, and the amino acid sequence set forth in SEQ ID NO:1. In certain embodiments, the CARs provided herein comprise the amino acid sequence set forth at residues 172-278 of SEQ ID NO:4 or a portion thereof, the amino acid sequence set forth in SEQ ID NO:6, and the amino acid sequence set forth in SEQ ID NO:1.

In certain embodiments, the CARs provided herein comprise (a) a TMIGD2 costimulatory domain, (b) an intracellular CD3ζ effector domain or a portion or variant thereof, (c) a CD8α transmembrane region or a portion or variant thereof, and (d) a CD8α hinge region or a portion or variant thereof. In certain of these embodiments (a) the TMIGD2 costimulatory domain comprises, consists of, or consists essentially of the amino acid sequence set forth at residues 172-282 of SEQ ID NO:3 or a portion thereof, residues 52-162 of SEQ ID NO:5 or a portion thereof, or residues 172-278 of SEQ ID NO: 4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; (b) the CD3ζ effector domain comprises, consists of, or consists essentially of the amino acid sequence set forth in SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO:6 or a portion thereof; (c) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof; and (d) the CD8α hinge region comprises, consists of, or consists essentially of the amino acid sequence set forth in SEQ ID NO:2 or a portion thereof, or a sequence with at least 75%, 80%, 85%, 90%, 91%, 92%, 93%, 94%, 95%, 96%, 97%, 98%, or 99% identity to SEQ ID NO:2 or a portion thereof. In certain embodiments, the TMIGD2 costimulatory domain is located between the CD8α transmembrane region and the CD3ζ effector domain. In other embodiments, the CD3ζ effector domain is located between the CD8α transmembrane region and the TMIGD2 costimulatory domain. In certain embodiments, the CD8α transmembrane region is directly adjacent to the CD3ζ effector domain or TMIGD2 costimulatory domain, while in other embodiments there are one or more amino acids separating the CD8α transmembrane region and the CD3ζ effector domain or TMIGD2 costimulatory domain. Similarly, in certain embodiments the TMIGD2 costimulatory domain and CD3ζ effector domain are directly adjacent to one another, while in other embodiments there are one or more amino acids separating the TMIGD2 costimulatory domain and the intracellular CD3ζ effector domain. In certain embodiments, the CD8α hinge region is directly adjacent to the CD8α transmembrane region. In other embodiments, there are one or more amino acids separating the hinge region and the transmembrane region. In certain embodiments, the CARs provided herein comprise the amino acid sequence set forth at residues 172-282 of SEQ ID NO:3 or a portion thereof, the amino acid sequence set forth in SEQ ID NO:6, the amino acid sequence set forth in SEQ ID NO:1, and the amino acid sequence set forth in SEQ ID NO:2. In certain embodiments, the CARs provided herein comprise the amino acid sequence set forth at residues 52-162 of SEQ ID NO:5 or a portion thereof, the amino acid sequence set forth in SEQ ID NO:6, the amino acid sequence set forth in SEQ ID NO:1, and the amino acid sequence set forth in SEQ ID NO: 2. In certain embodiments, the CARs provided herein comprise the amino acid sequence set forth at residues 172-278 of SEQ ID NO:4 or a portion thereof, the amino acid sequence set forth in SEQ ID NO:6, the amino acid sequence set forth in SEQ ID NO: 1, and the amino acid sequence set forth in SEQ ID NO:2.

In certain embodiments, the CARs provided herein comprise (a) a TMIGD2 costimulatory domain, (b) a CD8α transmembrane region or a portion or variant thereof, and (c) a CD8α hinge region. In certain of these embodiments the TMIGD2 costimulatory domain comprises, consists of, or consists essentially of the amino acid sequence set forth at residues 172-282 of SEQ ID NO:3 or a portion thereof, residues 52-162 of SEQ ID NO: 5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO: 4 or a portion thereof; (b) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof; and (c) the CD8α hinge region comprises, consists of, or consists essentially of the amino acid sequence set forth in SEQ ID NO:2 or a portion thereof, or a sequence with at least 75%, 80%, 85%, 90%, 91%, 92%, 93%, 94%, 95%, 96%, 97%, 98%, or 99% identity to SEQ ID NO:2 or a portion thereof. In certain embodiments, the CD8α transmembrane region is directly adjacent to the TMIGD2 costimulatory domain, while in other embodiments there are one or more amino acids separating the CD8α transmembrane region and the TMIGD2 costimulatory domain. In certain embodiments, the CD8α hinge region is directly adjacent to the CD8α transmembrane region. In other embodiments, there are one or more amino acids separating the hinge region and the transmembrane region. In certain embodiments, the CARs provided herein comprise the amino acid sequence set forth at residues 172-282 of SEQ ID NO:3 or a portion thereof, the amino acid sequence set forth in SEQ ID NO:1, and the amino acid sequence set forth in SEQ ID NO:2. In certain embodiments, the CARs provided herein comprise the amino acid sequence set forth at residues 52-162 of SEQ ID NO:5 or a portion thereof, the amino acid sequence set forth in SEQ ID NO:1, and the amino acid sequence set forth in SEQ ID NO:2. In certain embodiments, the CARs provided herein comprise the amino acid sequence set forth at residues 172-278 of SEQ ID NO:4 or a portion thereof, the amino acid sequence set forth in SEQ ID NO:1, and the amino acid sequence set forth in SEQ ID NO:2.

In certain embodiments, the CARs provided herein comprise (a) a TMIGD2 costimulatory domain, (b) an intracellular CD3ζ effector domain or a portion or variant thereof, (c) a CD8α transmembrane region or a portion or variant thereof, and (d) an extracellular region comprising an antigen binding domain. In certain of these embodiments (a) the TMIGD2 costimulatory domain comprises, consists of, or consists essentially of the amino acid sequence set forth at residues 172-282 of SEQ ID NO:3 or a portion thereof, residues 52-162 of SEQ ID NO:5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; (b) the CD3ζ effector domain comprises, consists of, or consists essentially of the amino acid sequence set forth in SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO:6 or a portion thereof; (c) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof; and (d) the antigen binding domain is an scFv that specifically binds a tumor-associated antigen. In certain embodiments, the TMIGD2 costimulatory domain is located between the CD8α transmembrane region and the CD3ζ effector domain. In other embodiments, the CD3ζ effector domain is located between the CD8α transmembrane region and the TMIGD2 costimulatory domain. In certain embodiments, the CD8α transmembrane region is directly adjacent to the CD3ζ effector domain or TMIGD2 costimulatory domain, while in other embodiments there are one or more amino acids separating the CD8α transmembrane region and the CD3ζ effector domain or TMIGD2 costimulatory domain. Similarly, in certain embodiments the TMIGD2 costimulatory domain and CD3ζ effector domain are directly adjacent to one another, while in other embodiments there are one or more amino acids separating the TMIGD2 costimulatory domain and the intracellular CD3ζ effector domain. Likewise, in certain embodiments the antigen binding domain is directly adjacent to the CD8α transmembrane region, while in other embodiments there are one or more amino acids separating the antigen binding domain and the CD8α transmembrane region. In certain of these embodiments, the amino acids separating the antigen binding domain and the CD8α transmembrane region comprise a hinge region, and in certain of these embodiments the hinge region is a CD8 hinge region comprising, consisting of, or consisting essentially of the amino acid sequence set forth in SEQ ID NO: 2 or a portion thereof, or a sequence with at least 75%, 80%, 85%, 90%, 91%, 92%, 93%, 94%, 95%, 96%, 97%, 98%, or 99% identity to SEQ ID NO:2 or a portion thereof.

In certain embodiments, the CARs provided herein comprise (a) a TMIGD2 costimulatory domain, (b) a CD8α transmembrane region or a portion or variant thereof, and (c) an extracellular region comprising an antigen binding domain. In certain of these embodiments (a) the TMIGD2 costimulatory domain comprises, consists of, or consists essentially of the amino acid sequence set forth at residues 172-282 of SEQ ID NO:3 or a portion thereof, residues 52-162 of SEQ ID NO:5 or a portion thereof, or residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or a portion thereof, residues 52-162 of SEQ ID NO:5 or a portion thereof, or residues 172-278 of SEQ ID NO:4 or a portion thereof; (b) the CD8α transmembrane region comprises, consists of, or consists essentially of the amino acid sequence set forth in SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof; and (c) the antigen binding domain is an scFv that specifically binds a tumor-associated antigen. In certain embodiments, the CD8α transmembrane region is directly adjacent to the TMIGD2 costimulatory domain, while in other embodiments there are one or more amino acids separating the CD8α transmembrane region and the TMIGD2 costimulatory domain. Similarly, in certain embodiments the antigen binding domain is directly adjacent to the CD8α transmembrane region, while in other embodiments there are one or more amino acids separating the antigen binding domain and the CD8α transmembrane region. In certain of these embodiments, the amino acids separating the antigen binding domain and the CD8α transmembrane region comprise a hinge region, and in certain of these embodiments the hinge region is a CD8α hinge region comprising, consisting of, or consisting essentially of the amino acid sequence set forth in SEQ ID NO: 2 or a portion thereof, or a sequence with at least 75%, 80%, 85%, 90%, 91%, 92%, 93%, 94%, 95%, 96%, 97%, 98%, or 99% identity to SEQ ID NO:2 or a portion thereof.

In certain embodiments, the CARs provided herein comprise the amino acid sequence set forth at residues 172-282 of SEQ ID NO:3 or a portion thereof, the amino acid sequence set forth in SEQ ID NO:6, the amino acid sequence set forth in SEQ ID NO: 1, the amino acid sequence set forth in SEQ ID NO:2, and an antigen binding domain. In certain embodiments, the CARs provided herein comprise the amino acid sequence set forth at residues 52-162 of SEQ ID NO:5 or a portion thereof, the amino acid sequence set forth in SEQ ID NO:6, the amino acid sequence set forth in SEQ ID NO:1, the amino acid sequence set forth in SEQ ID NO:2, and an antigen binding domain. In certain embodiments, the CARs provided herein comprise the amino acid sequence set forth at residues 172-278 of SEQ ID NO:4 or a portion thereof, the amino acid sequence set forth in SEQ ID NO:6, the amino acid sequence set forth in SEQ ID NO:1, the amino acid sequence set forth in SEQ ID NO:2, and an antigen binding domain.

TABLE 1
Exemplary CAR Components

| SEQ | | |
|---|---|---|
| ID NO. | NAME | SEQUENCE |
| 1 | CD8α | YIWAPLAGTCGVLLLSLVITLYC |
| | transmembrane | |
| | region | |
| 2 | CD8α hinge | TTTPAPRPPTPAPTIASQPLSLRPEACRPAAGGAVHTRG |
| | region | LDFACDI |
| 3 | TMIGD2 - | MGSPGMVLGLLVQIWALQEASSLSVQQGPNLLQVRQGSQ |
| | Isoform 1* | ATLVCQVDQATAWERLRVKWTKDGAILCQPYITNGSLSL |
| | | GVCGPQGRLSWQAPSHLTLQLDPVSLNHSGAYVCWAAVE |
| | | IPELEEAEGNITRLFVDPDDPTQNRNRIASFPGFLFVLL |
| | | GVGSMGVAAIVWGAWFWGRRSCQQRDSGNSPGNAFYSNV |
| | | LYRPRGAPKKSEDCSGEGKDQRGQSIYSTSFPQPAPRQP |
| | | HLASRPCPSPRPCPSPRPGHPVSMVRVSPRPSPTQQPRP |
| | | KGFPKVGEE |
| 4 | TMIGD2 - | MGSPGMVLGLLVQIWALQEASSLSVQQGPNLLQVRQGSQ |
| | Isoform 2* | ATLVCQVDQATAWERLRVKWTKDGAILCQPYITNGSLSL |
| | | GVCGPQGRLSWQAPSHLTLQLDPVSLNHSGAYVCWAAVE |
| | | IPELEEAEGNITRLFVDPDDPTQNRNRIASFPGFLFVLL |
| | | GVGSMGVAAIVWGAWFWGRRSCQQRDSGNAFYSNVLYRP |
| | | RGAPKKSEDCSGEGKDQRGQSIYSTSFPQPAPRQPHLAS |
| | | RPCPSPRPCPSPRPGHPVSMVRVSPRPSPTQQPRPKGFP |
| | | KVGEE |
| 5 | TMIGD2 - | MGSPGMVLGLLVQIWDDPTQNRNRIASFPGFLFVLLGVG |
| | Isoform 3* | SMGVAAIVWGAWFWGRRSCQQRDSGNSPGNAFYSNVLYR |
| | | PRGAPKKSEDCSGEGKDQRGQSIYSTSFPQPAPRQPHLA |
| | | SRPCPSPRPCPSPRPGHPVSMVRVSPRPSPTQQPRPKGF |
| | | PKVGEE |
| 6 | CD3ζ | RVKFSRSADAPAYQQGQNQLYNELNLGRREEYDVLDKRR |
| | | GRDPEMGGKPQRRKNPQEGLYNELQKDKMAEAYSEIGMK |
| | | GERRRGKGHDGLYQGLSTATKDTYDALHMQALPPR |

*Intracellular regions of TMIGD2 isoforms 1, 2, and 3 are underlined.

### Nucleic Acid Molecules, Vectors, and Cells

Provided herein in certain embodiments are nucleic acid molecules encoding any one or more of the CARs provided herein, as well as compositions and vectors comprising these nucleic acid molecules. Also provided herein are cells comprising these nucleic acid molecules or vectors and compositions thereof, and unit doses of these cells and compositions. In some embodiments, these unit doses comprise (i) a composition comprising at least about 50% modified CD4+ T cells, combined with (ii) a composition comprising at least about 50% modified CD8+ T cells, in about a 1:1 ratio, wherein the unit dose contains a reduced amount or substantially no naïve T cells.

### TMIGD2 Costimulatory Domain

In certain embodiments, the nucleic acid molecules provided herein encode a CAR comprising a TMIGD2 costimulatory domain derived from TMIGD2 isoform 1 (SEQ ID NO:9), isoform 2 (SEQ ID NO:10), or isoform 3 (SEQ ID NO:11).

In certain embodiments, the nucleic acid molecules provided herein encode residues 172-282 of SEQ ID NO:3 or a portion thereof, residues 52-162 of SEQ ID NO:5 or a portion thereof, or a sequence with at least 75%, 80%, 85%, 90%, 91%, 92%, 93%, 94%, 95%, 96%, 97%, 98%, or 99% identity to residues 172-282 of SEQ ID NO:3 or residues 52-162 of SEQ ID NO:5 or a portion thereof. In certain of these embodiments, the nucleic acid molecules comprise, consist of, or consist essentially of nucleotides 514-849 of SEQ ID NO:9 or a portion thereof, nucleotides 154-489 of SEQ ID NO:11 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or nucleotides 154-489 of SEQ ID NO:11 or a portion thereof.

In certain embodiments, the nucleic acid molecules provided herein encode residues 172-278 of SEQ ID NO:4 or a portion thereof, or a sequence with at least 75%, 80%, 85%, 90%, 91%, 92%, 93%, 94%, 95%, 96%, 97%, 98%, or 99% identity to residues 172-278 of SEQ ID NO:4 or a portion thereof. In certain of these embodiments, the nucleic acid molecules comprise, consist of, or consist essentially of nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-837 of SEQ ID NO:10 or a portion thereof.

### Intracellular Effector Domain

In certain embodiments, the nucleic acid molecules provided herein encode a CAR comprising an intracellular effector domain. In some embodiments, the intracellular effector domain is a CD3ζ effector domain or a functional portion or variant thereof.

In certain embodiments, the nucleic acid molecules provided herein encode the amino acid sequence of SEQ ID NO:6 or a portion thereof, or a sequence with at least 75%, 80%, 85%, 90%, 91%, 92%, 93%, 94%, 95%, 96%, 97%, 98%, or 99% identity to SEQ ID NO:6 or a portion thereof. In certain of these embodiments, the nucleic acid molecules comprise, consist of, or consist essentially of the nucleotide sequence set forth in SEQ ID NO:12 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:12 or a portion thereof.

### Transmembrane Region

In certain embodiments, the nucleic acid molecules provided herein encode a CAR comprising a transmembrane domain. In some embodiments, the transmembrane domain is a CD8α transmembrane domain or a variant thereof.

In certain embodiments, the nucleic acid molecules provided herein encode the amino acid sequence of SEQ ID NO:1 or a portion thereof, or a sequence with at least 75%, 80%, 85%, 90%, 91%, 92%, 93%, 94%, 95%, 96%, 97%, 98%, or 99% identity to SEQ ID NO:1 or a portion thereof. In certain of these embodiments, the nucleic acid molecules comprise, consist of, or consist essentially of the nucleotide sequence set forth in SEQ ID NO:7 or a portion thereof, or a sequence with at least 75%, 80%, 85%, 90%, 91%, 92%, 93%, 94%, 95%, 96%, 97%, 98%, or 99% identity to SEQ ID NO:7.

### Extracellular Region

In certain embodiments, the nucleic acid molecules provided herein comprise a nucleic acid sequence encoding an extracellular region. In certain of these embodiments the extracellular region comprises an antigen binding domain, and in certain of these embodiments the antigen binding domain specifically binds a tumor-associated antigen selected from HHLA2, CD19; CD20; BCMA; CD22; CD3; CEACAM6; c-Met; EGFR; EGFRvIII; ErbB2; ErbB3; ErbB4; EphA2; IGF1R; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTβR; LIFRβ; LRP5; MUC1; OSMRβ; TCRα; TCRβ; CD25; CD28; CD30; CD33; CD52; CD56; CD79a; CD79b; CD80; CD81; CD86; CD123; CD171; CD276; B7-H3; B7H4; TLR7; TLR9; PTCH1; WT-1; HA1-H; Robo1; α-fetoprotein (AFP); Frizzled; OX40; PRAME; and/or SSX-2 antigen.

In certain embodiments, the extracellular region further comprises a hinge region that connects the antigen binding domain to a transmembrane domain. In certain of these embodiments, the hinge region is a CD8α hinge region. In certain embodiments, the nucleic acid molecules provided herein encode the CD8α hinge region of SEQ ID NO: 2 or a portion thereof, or a sequence with at least 75%, 80%, 85%, 90%, 91%, 92%, 93%, 94%, 95%, 96%, 97%, 98%, or 99% identity to SEQ ID NO:2 or a portion thereof. In certain of these embodiments, the nucleic acid molecules comprise, consist of, or consist essentially of the nucleotide sequence set forth in SEQ ID NO:8 or a portion thereof, or a sequence with at least 75%, 80%, 85%, 90%, 91%, 92%, 93%, 94%, 95%, 96%, 97%, 98%, or 99% identity to SEQ ID NO:8.

### Markers and Self-Cleaving Peptides

In certain embodiments, the nucleic acid molecules provided herein comprise a nucleotide sequence encoding an encoding marker, such as GFP and/or EGFRt. In certain of these embodiments, the nucleic acid molecules encode the EGFRt encoding marker of SEQ ID NO:15 or a portion thereof, or a sequence with at least 75%, 80%, 85%, 90%, 91%, 92%, 93%, 94%, 95%, 96%, 97%, 98%, or 99% identity to SEQ ID NO: 15 or a portion thereof. In certain of these embodiments, the nucleic acid molecules comprise, consist of, or consist essentially of the nucleotide sequence set forth in SEQ ID NO:18 or a portion thereof, or a sequence with at least 70%, 75%, 80%, 85%, 90%, 91%, 92%, 93%, 94%, 95%, 96%, 97%, 98%, 99%, or more identity to SEQ ID NO. 18.

In certain embodiments, the nucleic acid molecules provided herein comprise a nucleotide sequence encoding a self-cleaving peptide, such as a 2A peptide. Exemplary 2A peptides are P2A and F2A.

In certain of these embodiments, the nucleic acid molecules encode the P2A self-cleaving peptide of SEQ ID NO:13 or a portion thereof or SEQ ID NO:14 or portion thereof, or a sequence with at least 75%, 80%, 85%, 90%, 91%, 92%, 93%, 94%, 95%, 96%, 97%, 98%, or 99% identity to SEQ ID NO:13 or a portion thereof or SEQ ID NO:14 or portion thereof. In certain of these embodiments, the nucleic acid molecules comprise, consist of, or consist essentially of the nucleotide sequence set forth in SEQ ID NO:16 or a portion thereof or SEQ ID NO:17 or portion thereof, or a sequence with at least 70%, 75%, 80%, 85%, 90%, 91%, 92%, 93%, 94%, 95%, 96%, 97%, 98%, 99%, or more identity to SEQ ID NO. 16 or SEQ ID NO:17.

In certain embodiments, the nucleic acid molecules provided herein comprising a nucleotide sequence encoding an encoding marker, such as GFP and/or EGFRt are attached to a signal leader sequence. One exemplar sequence is the GMCSFR alpha chain signal sequence, which directs surface expression, attached to EGFRt.

### SPECIFIC EMBODIMENTS

In certain embodiments, the nucleic acid molecules provided herein encode (a) a TMIGD2 costimulatory domain and (b) an intracellular CD3ζ effector domain or a portion or variant thereof. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO: 11 or a portion thereof, or nucleotides 514-837 of SEQ ID NO:10 or a portion thereof; and (b) SEQ ID NO:12 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:12 or a portion thereof. In certain embodiments, the nucleic acid sequence encoding the TMIGD2 costimulatory domain and the nucleic acid sequence encoding the CD3ζ effector domain are directly adjacent to one another, while in other embodiments there are one or more nucleotides separating the nucleic acid sequences encoding the TMIGD2 costimulatory domain and the CD3ζ effector domain. In certain embodiments, the nucleic acid molecules further comprise, consist, or consist essentially of SEQ ID NO:16 or a portion thereof or SEQ ID NO:17 or a portion thereof, SEQ ID NO:18 or a portion thereof, both SEQ ID NO:16 and SEQ ID NO: 18 or portions thereof, or both SEQ ID NO:17 and SEQ ID NO:18 or a portion thereof.

In certain embodiments, the nucleic acid molecules provided herein encode (a) a TMIGD2 costimulatory domain and (b) a CD8α transmembrane region or a portion or variant thereof or a portion or variant thereof. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof; and (b) SEQ ID NO:7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:7 or a portion thereof. In certain embodiments, the nucleic acid sequence encoding the TMIGD2 costimulatory domain and the nucleic acid sequence encoding the CD8α transmembrane region are directly adjacent to one another, while in other embodiments there are one or more nucleotides separating the nucleic acid sequences encoding the TMIGD2 costimulatory domain and the CD8α transmembrane region. In certain embodiments, the nucleic acid molecules further comprise, consist, or consist essentially of SEQ ID NO: 16 or a portion thereof or SEQ ID NO:17 or a portion thereof, SEQ ID NO:18 or a portion thereof, both SEQ ID NO:16 and SEQ ID NO:18 or portions thereof, or both SEQ ID NO: 17 and SEQ ID NO:18 or a portion thereof.

In certain embodiments, the nucleic acid molecules provided herein encode (a) a TMIGD2 costimulatory domain, (b) an intracellular CD3ζ effector domain or a portion or variant thereof, and (c) a CD8α transmembrane region or a portion or variant thereof or a portion or variant thereof. In certain of these embodiments, the nucleic acid molecules comprise, consist of, or consist essentially of (a) nucleotides 514-849 of SEQ ID NO: 9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO: 11 or a portion thereof, or nucleotides 514-837 of SEQ ID NO:10 or a portion thereof; (b) SEQ ID NO:12 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:12 or a portion thereof; and (c) SEQ ID NO:7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO: 7 or a portion thereof. In certain embodiments, the nucleic acid sequence encoding the TMIGD2 costimulatory domain is located between the nucleic acid sequences encoding the CD8α transmembrane region and the CD3ζ effector domain. In other embodiments, the nucleic acid sequence encoding the CD3ζ effector domain is located between the nucleic acid sequences encoding the CD8α transmembrane region and the TMIGD2 costimulatory domain. In certain embodiments, the nucleic acid sequence encoding the CD8α transmembrane region is directly adjacent to the nucleic acid sequence encoding the CD3ζ effector domain or TMIGD2 costimulatory domain, while in other embodiments there are one or more nucleotides separating the nucleic acid sequences encoding the CD8α transmembrane region and the CD3ζ effector domain or TMIGD2 costimulatory domain. Similarly, in certain embodiments the nucleic acid sequences encoding the TMIGD2 costimulatory domain and CD3ζ effector domain are directly adjacent to one another, while in other embodiments there are one or more nucleotides separating the nucleic acid sequences encoding the TMIGD2 costimulatory domain and the intracellular CD3ζ effector domain. In certain embodiments, the nucleic acid molecules further comprise, consist, or consist essentially of SEQ ID NO:16 or a portion thereof or SEQ ID NO:17 or a portion thereof, SEQ ID NO:18 or a portion thereof, both SEQ ID NO:16 and SEQ ID NO:18 or portions thereof, or both SEQ ID NO:17 and SEQ ID NO:18 or a portion thereof.

In certain embodiments, the nucleic acid molecules provided herein encode (a) a TMIGD2 costimulatory domain, (b) an intracellular CD3ζ effector domain or a portion or variant thereof, (c) a CD8α transmembrane region or a portion or variant thereof or a portion or variant thereof, and (d) a CD8α hinge region or a portion or variant thereof. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO:11 or a portion thereof, or nucleotides 514-837 of SEQ ID NO: 10 or a portion thereof; (b) SEQ ID NO:12 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:12 or a portion thereof; (c) SEQ ID NO:7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:7 or a portion thereof, and (d) SEQ ID NO: 8 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:8 or a portion thereof. In certain embodiments, the nucleic acid sequence encoding the TMIGD2 costimulatory domain is located between the nucleic acid sequences encoding the CD8α transmembrane region and the CD3ζ effector domain. In other embodiments, the nucleic acid sequence encoding the CD3ζ effector domain is located between the nucleic acid sequences encoding the CD8α transmembrane region and the TMIGD2 costimulatory domain. In certain embodiments, the nucleic acid sequence encoding the CD8α transmembrane region is directly adjacent to the nucleic acid sequence encoding the CD3ζ effector domain or TMIGD2 costimulatory domain, while in other embodiments there are one or more nucleotides separating the nucleic acid sequences encoding the CD8α transmembrane region and the CD3ζ effector domain or TMIGD2 costimulatory domain. Similarly, in certain embodiments the nucleic acid sequences encoding the TMIGD2 costimulatory domain and CD3ζ effector domain are directly adjacent to one another, while in other embodiments there are one or more nucleotides separating the nucleic acid sequences encoding the TMIGD2 costimulatory domain and the intracellular CD3ζ effector domain. In certain embodiments, nucleic acid sequences encoding the CD8α hinge region are directly adjacent to the nucleic acid sequences encoding the CD8α transmembrane region, whereas in other embodiments there are one or more nucleotides separating the nucleic acid sequences encoding the hinge region and the transmembrane region. In certain embodiments, the nucleic acid molecules further comprise, consist, or consist essentially of SEQ ID NO:16 or a portion thereof or SEQ ID NO:17 or a portion thereof, SEQ ID NO:18 or a portion thereof, both SEQ ID NO:16 and SEQ ID NO:18 or portions thereof, or both SEQ ID NO:17 and SEQ ID NO: 18 or a portion thereof.

In certain embodiments, the nucleic acid molecules provided herein encode (a) a TMIGD2 costimulatory domain, (b) a CD8α transmembrane region or a portion or variant thereof or a portion or variant thereof, and (c) a CD8α hinge region or a portion or variant thereof. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof; (b) SEQ ID NO: 7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:7 or a portion thereof, and (c) SEQ ID NO:8 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:8 or a portion thereof. In certain embodiments, the nucleic acid sequence encoding the TMIGD2 costimulatory domain is located between the nucleic acid sequences encoding the CD8α transmembrane region and the CD3ζ effector domain. In other embodiments, the nucleic acid sequence encoding the CD3ζ effector domain is located between the nucleic acid sequences encoding the CD8α transmembrane region and the TMIGD2 costimulatory domain. In certain embodiments, the nucleic acid sequence encoding the CD8α transmembrane region is directly adjacent to the nucleic acid sequence encoding the CD3ζ effector domain or TMIGD2 costimulatory domain, while in other embodiments there are one or more nucleotides separating the nucleic acid sequences encoding the CD8α transmembrane region and the CD3ζ effector domain or TMIGD2 costimulatory domain. Similarly, in certain embodiments the nucleic acid sequences encoding the TMIGD2 costimulatory domain and CD3ζ effector domain are directly adjacent to one another, while in other embodiments there are one or more nucleotides separating the nucleic acid sequences encoding the TMIGD2 costimulatory domain and the intracellular CD3ζ effector domain. In certain embodiments, nucleic acid sequences encoding the CD8α hinge region are directly adjacent to the nucleic acid sequences encoding the CD8α transmembrane region, whereas in other embodiments there are one or more nucleotides separating the nucleic acid sequences encoding the hinge region and the transmembrane region. In certain embodiments, the nucleic acid molecules further comprise, consist, or consist essentially of SEQ ID NO:16 or a portion thereof or SEQ ID NO: 17 or a portion thereof, SEQ ID NO:18 or a portion thereof, both SEQ ID NO:16 and SEQ ID NO:18 or portions thereof, or both SEQ ID NO:17 and SEQ ID NO:18 or a portion thereof.

In certain embodiments, the nucleic acid molecules provided herein encode (a) a TMIGD2 costimulatory domain, (b) an intracellular CD3ζ effector domain or a portion or variant thereof, (c) a CD8α transmembrane region or a portion or variant thereof or a portion or variant thereof, and (d) an extracellular region comprising an antigen binding domain. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof; (b) SEQ ID NO:12 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO: 12 or a portion thereof; (c) SEQ ID NO:7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:7 or a portion thereof, and (d) the antigen binding domain is an scFv that specifically binds a tumor-associated antigen. In certain embodiments, the nucleic acid sequence encoding the TMIGD2 costimulatory domain is located between the nucleic acid sequences encoding the CD8α transmembrane region and the CD3ζ effector domain. In other embodiments, the nucleic acid sequence encoding the CD3ζ effector domain is located between the nucleic acid sequences encoding the CD8α transmembrane region and the TMIGD2 costimulatory domain. In certain embodiments, the nucleic acid sequence encoding the CD8α transmembrane region is directly adjacent to the nucleic acid sequence encoding the CD3ζ effector domain or TMIGD2 costimulatory domain, while in other embodiments there are one or more nucleotides separating the nucleic acid sequences encoding the CD8α transmembrane region and the CD3ζ effector domain or TMIGD2 costimulatory domain. Similarly, in certain embodiments the nucleic acid sequences encoding the TMIGD2 costimulatory domain and CD3ζ effector domain are directly adjacent to one another, while in other embodiments there are one or more nucleotides separating the nucleic acid sequences encoding the TMIGD2 costimulatory domain and the intracellular CD3ζ effector domain. Likewise, in certain embodiments the antigen binding domain is directly adjacent to the CD8α transmembrane region, while in other embodiments there are one or more nucleotides separating the antigen binding domain and the CD8α transmembrane region. In certain of these embodiments, the nucleotides separating the antigen binding domain and the CD8α transmembrane region comprise a hinge region and in certain of these embodiments the hinge region is a CD8α hinge region comprising, consisting of, or consisting essentially of the nucleotide sequence set forth in SEQ ID NO: 8 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:8 or a portion thereof. In certain embodiments, the nucleic acid molecules further comprise, consist, or consist essentially of SEQ ID NO:16 or a portion thereof or SEQ ID NO:17 or a portion thereof, SEQ ID NO:18 or a portion thereof, both SEQ ID NO: 16 and SEQ ID NO: 18 or portions thereof, or both SEQ ID NO:17 and SEQ ID NO:18 or a portion thereof.

In certain embodiments, the nucleic acid molecules provided herein encode (a) a TMIGD2 costimulatory domain, (b) a CD8α transmembrane region or a portion or variant thereof or a portion or variant thereof, and (c) an extracellular region comprising an antigen binding domain. In certain of these embodiments, the nucleic acid molecules comprise, consist of, or consist essentially of (a) nucleotides 514-849 of SEQ ID NO:9 or a portion thereof, nucleotides 154-489 of SEQ ID NO:11 or a portion thereof, or nucleotides 514-837 of SEQ ID NO:10 or a portion thereof, or a sequence with at least 75%, 80%, 85%, 90%, 91%, 92%, 93%, 94%, 95%, 96%, 97%, 98%, or 99% identity to nucleotides 514-849 of SEQ ID NO:9 or a portion thereof, nucleotides 154-489 of SEQ ID NO: 11 or a portion thereof, or nucleotides 514-837 of SEQ ID NO:10 or a portion thereof; (b) SEQ ID NO:7 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:7 or a portion thereof, and (c) the antigen binding domain is an scFv that specifically binds a tumor-associated antigen. In certain embodiments, the nucleic acid sequence encoding the TMIGD2 costimulatory domain and the nucleic acid sequence encoding the CD8α transmembrane region are directly adjacent to one another, while in other embodiments there are one or more nucleotides separating the nucleic acid sequences encoding the TMIGD2 costimulatory domain and the CD8α transmembrane region. In certain embodiments the antigen binding domain is directly adjacent to the CD8α transmembrane region, while in other embodiments there are one or more nucleotides separating the antigen binding domain and the CD8α transmembrane region. In certain of these embodiments, the nucleotides separating the antigen binding domain and the CD8α transmembrane region comprise a hinge region and in certain of these embodiments the hinge region is a CD8α hinge region comprising, consisting of, or consisting essentially of the nucleotide sequence set forth in SEQ ID NO: 8 or a portion thereof, or a sequence with at least 75%, 80%, 85%, 90%, 91%, 92%, 93%, 94%, 95%, 96%, 97%, 98%, or 99% identity to the nucleotide sequence set forth in SEQ ID NO:8 or a portion thereof. In certain embodiments, the nucleic acid molecules further comprise, consist, or consist essentially of SEQ ID NO:16 or a portion thereof or SEQ ID NO:17 or a portion thereof, SEQ ID NO:18 or a portion thereof, both SEQ ID NO: 16 and SEQ ID NO:18 or portions thereof, or both SEQ ID NO: 17 and SEQ ID NO:18 or a portion thereof.

In certain embodiments, the nucleic acid molecules provided herein comprise the nucleotide sequence set forth at nucleotides 514-849 of SEQ ID NO:9 or a portion thereof, the nucleotide sequence set forth in SEQ ID NO:12, the nucleotide sequence set forth in SEQ ID NO:7, the nucleotide sequence set forth in SEQ ID NO:8, and a nucleic acid sequence encoding an antigen binding domain. In certain embodiments, the nucleic acid molecules provided herein comprise the nucleotide sequence set forth at nucleotides 514-837 of SEQ ID NO:10 or a portion thereof, the nucleotide sequence set forth in SEQ ID NO:12, the nucleotide sequence set forth in SEQ ID NO:7, the nucleotide sequence set forth in SEQ ID NO:8, and a nucleic acid sequence encoding an antigen binding domain. In certain embodiments, the nucleic acid molecules provided herein comprise the nucleotide sequence set forth at nucleotides 154-489 of SEQ ID NO:11 or a portion thereof, the nucleotide sequence set forth in SEQ ID NO: 12, the nucleotide sequence set forth in SEQ ID NO:7, the nucleotide sequence set forth in SEQ ID NO:8, and a nucleic acid sequence encoding an antigen binding domain.

TABLE 2
Exemplary CAR Components

| SEQ | | |
|---|---|---|
| ID NO. | NAME | SEQUENCE |
| 7 | CD8α | tacatctgggctccactggcaggaacctgtggcgtgctgctgctgtccctggt |
| | transmembrane | catcacactttactgc |
| | region | |
| 8 | CD8α hinge | accacaactcctgctccaagaccccctacacccgcccctactatcgcttctca |
| | region | gcctctgagtctgagaccagaggcttgcaggcccgcagctggaggagcagtgc |
| | | acactaggggcctggacttcgcctgcgacatc |
| 9 | TMIGD2 - | atggggtccccgggcatggtgctgggcctcctggtgcagatctgggccctgca |
| | Isoform 1* | agaagcctcaagcctgagcgtgcagcaggggcccaacttgctgcaggtgaggc |
| | | agggcagtcaggcgaccctggtctgccaggtggaccaggccacagcctgggaa |
| | | cggctccgtgttaagtggacaaaggatggggccatcctgtgtcaaccgtacat |
| | | caccaacggcagcctcagcctgggggtctgcgggccccagggacggctctcct |
| | | ggcaggcacccagccatctcaccctgcagctggaccctgtgagcctcaaccac |
| | | agcggggcgtacgtgtgctgggcggccgtagagattcctgagttggaggaggc |
| | | tgagggcaacataacaaggctctttgtggacccagatgaccccacacagaaca |
| | | gaaaccggatcgcaagcttcccaggattcctcttcgtgctgctggggggggaa |
| | | gcatgggtgtggctgcgatcgtgtggggtgcctggttctggggccgccgcagc |
| | | tgccagcaaagggactcaggtaacagcccaggaaatgcattctacagcaacgt |
| | | cctataccggccccggggggccccaaagaagagtgaggactgctctggagagg |
| | | ggaaggaccagaggggccagagcatttattcaacctccttcccgcaaccggcc |
| | | ccccgccagccgcacctggcgtcaagaccctgccccagcccgagaccctgccc |
| | | cagccccaggcccggccaccccgtctctatggtcagggtctctcctagaccaa |
| | | gccccacccagcagccgaggccaaaagggttccccaaagtgggagaggagtga |
| 10 | TMIGD2 - | atggggtccccgggcatggtgctgggcctcctggtgcagatctgggccctgca |
| | Isoform 2* | agaagcctcaagcctgagcgtgcagcaggggcccaacttgctgcaggtgaggc |
| | | agggcagtcaggcgaccctggtctgccaggtggaccaggccacagcctgggaa |
| | | cggctccgtgttaagtggacaaaggatggggccatcctgtgtcaaccgtacat |
| | | caccaacggcagcctcagcctgggggtctgcgggccccagggacggctctcct |
| | | ggcaggcacccagccatctcaccctgcagctggaccctgtgagcctcaaccac |
| | | agcggggcgtacgtgtgctgggcggccgtagagattcctgagttggaggaggc |
| | | tgagggcaacataacaaggctctttgtggacccagatgaccccacacagaaca |
| | | gaaaccggatcgcaagcttcccaggattcctcttcgtgctgctggggggggaa |
| | | gcatgggtgtggctgcgatcgtgtggggtgcctggttctggggccgccgcagc |
| | | tgccagcaaagggactcaggaaatgcattctacagcaacgtcctataccggcc |
| | | cccggggggcccaaagaagagtgaggactgctctggagaggggaaggaccaga |
| | | ggggccagagcatttattcaacctccttcccgcaaccggccccccgccagccg |
| | | cacctggcgtcaagaccctgccccagcccgagaccctgccccagccccaggcc |
| | | cggccaccccgtctctatggtcagggtctctcctagaccaagccccacccagc |
| | | agccgaggccaaaagggttccccaaagtgggagaggagtga |
| 11 | TMIGD2 - | atggggtccccgggcatggtgctgggcctcctggtgcagatctgggatgaccc |
| | Isoform 3* | cacacagaacagaaaccggatcgcaagcttcccaggattcctcttcgtgctgc |
| | | tgggggtgggaagcatgggtgtggctgcgatcgtgtggggtgcctggttctgg |
| | | ggccgccgcagctgccagcaaagggactcaggtaacagcccaggaaatgcatt |
| | | ctacagcaacgtcctataccggccccggggggccccaaagaagagtgaggact |
| | | gctctggagaggggaaggaccagaggggccagagcatttattcaacctccttc |
| | | ccgcaaccggccccccgccagccgcacctggcgtcaagaccctgccccagccc |
| | | gagaccctgccccagccccaggcccggccaccccgtctctatggtcagggtct |
| | | ctcctagaccaagccccacccagcagccgaggccaaaagggttccccaaagtg |
| | | ggagaggagtga |
| 12 | CD3ζ | cgcgtcaagtttagccgatccgcagatgccccagcttaccagcagggacagaa |
| | | ccagctgtataacgagctgaatctgggccggagagaggaatacgacgtgctgg |
| | | acaaaaggcgcgggcgcgaccctgaaatgggaggaaagccacagcgacggaaa |
| | | aacccccaggagggactgtacaatgaactgcagaaggacaaaatggcagaggc |
| | | ctattcagaaattgggatgaagggagaaagaaggcgcggcaaagggcacgatg |
| | | gcctgtaccaggggctgagcactgccaccaaggacacctatgatgcactgcac |
| | | atgcaggccctgccccctcgc |

*Nucleic acids encoding the intracellular regions of TMIGD2 isoforms 1, 2, and 3 are underlined.

TABLE 3
Exemplary Cleaving Peptides and Encoding Markers

| SEQ | | |
|---|---|---|
| ID NO. | NAME | SEQUENCE |
| 13 | P2A | GSGATNFSLLKQAGDVEENPGP |
| 14 | P2A | ATNFSLLKQAGDVEENPGP |
| 15 | EGFRt | RKVCNGIGIGEFKDSLSINATNIKHFKNCTSISGDLHILPVAFRGDSFTHTPP |
| | | LDPQELDILKTVKEITGFLLIQAWPENRTDLHAFENLEIIRGRTKQHGQFSLA |
| | | VVSLNITSLGLRSLKEISDGDVIISGNKNLCYANTINWKKLFGTSGQKTKIIS |
| | | NRGENSCKATGQVCHALCSPEGCWGPEPRDCVSCRNVSRGRECVDKCNLLEGE |
| | | PREFVENSECIQCHPECLPQAMNITCTGRGPDNCIQCAHYIDGPHCVKTCPAG |
| | | VMGENNTLVWKYADAGHVCHLCHPNCTYGCTGPGLEGCPTNGPKIPSIATGMV |
| | | GALLLLLVVALGIGLFM |
| 16 | P2A | ggaagcggagctactaacttcagcctgctgaagcaggctggagacgtggagga |
| | | gaaccctggacct |
| 17 | P2A | gcaacaaacttctctctgctgaaacaagccggagatgtcgaagagaatcctgg |
| | | accg |
| 18 | EGFRt | cgcaaagtgtgtaacggaataggtattggtgaatttaaagactcactctccat |
| | | aaatgctacgaatattaaacacttcaaaaactgcacctccatcagtggcgatc |
| | | tccacatcctgccggtggcatttaggggtgactccttcacacatactcctcct |
| | | ctggatccacaggaactggatattctgaaaaccgtaaaggaaatcacagggtt |
| | | tttgctgattcaggcttggcctgaaaacaggacggacctccatgcctttgaga |
| | | acctagaaatcatacgcggcaggaccaagcaacatggtcagttttctcttgca |
| | | gtcgtcagcctgaacataacatccttgggattacgctccctcaaggagataag |
| | | tgatggagatgtgataatttcaggaaacaaaaatttgtgctatgcaaatacaa |
| | | taaactggaaaaaactgtttgggacctccggtcagaaaaccaaaattataagc |
| | | aacagaggtgaaaacagctgcaaggccacaggccaggtctgccatgccttgtg |
| | | ctcccccgagggctgctggggcccggagcccagggactgcgtctcttgccgga |
| | | atgtcagccgaggcagggaatgcgtggacaagtgcaaccttctggagggtgag |
| | | ccaagggagtttgtggagaactctgagtgcatacagtgccacccagagtgcct |
| | | gcctcaggccatgaacatcacctgcacaggacggggaccagacaactgtatcc |
| | | agtgtgcccactacattgacggcccccactgcgtcaagacctgcccggcagga |
| | | gtcatgggagaaaacaacaccctggtctggaagtacgcagacgccggccatgt |
| | | gtgccacctgtgccatccaaactgcacctacggatgcactgggccaggtcttg |
| | | aaggctgtccaacgaatgggcctaagatcccgtccatcgccactgggatggtg |
| | | ggggccctcctcttgctgctggtggtggccctggggatcggcctcttcatgta |
| | | a |

In any of the embodiments described herein, nucleic acid molecules encoding CARs may be codon-optimized for a particular cell using known techniques (Scholten et al., 2006). Codon optimization can be performed using, e.g., the GenScript® OptimumGene™ tool. Codon-optimized sequences include sequences that are partially or fully codon-optimized.

A nucleic acid molecule encoding a CAR of this disclosure can be inserted into an expression vector, such as a viral vector, for transduction into a cell, such as a T cell. In some embodiments, an expression construct of the present disclosure comprises a nucleic acid molecule encoding a CAR provided herein and, optionally, further encoding a self-cleaving peptide and/or EGFRt marker operably linked to an expression control sequence such as a promoter.

In certain embodiments, nucleic acid molecules of the present disclosure may be operatively linked to certain elements of the vector. For example, polynucleotide sequences that are needed to affect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the expression construct is comprised in a vector which may integrate into a cell's genome or promote integration of the nucleic acid molecule insert upon introduction into the cell and thereby replicate along with the cell's genome, such as a viral vector. Viral vectors include retrovirus, adenovirus, parvovirus, coronavirus, negative strand RNA viruses, positive strand RNA viruses, and double-stranded DNA viruses. (Coffin, J. M., Retroviridae: The viruses and their replication, In Fundamental Virology, Third Edition, B. N. Fields et al., Eds., Lippincott-Raven Publishers, Philadelphia, 1996).

Construction of an expression vector that is used for genetically engineering and producing a CAR of interest can be accomplished by using any suitable molecular biology engineering techniques known in the art. To obtain efficient transcription and translation, a polynucleotide in each recombinant expression construct includes at least one appropriate expression control sequence, such as a leader sequence and particularly a promoter operably linked to the nucleotide sequence encoding the immunogen. Methods for making CARs of the present disclosure are described, for example, in U.S. Pat. Nos. 6,410,319; 7,446,191; U.S. Patent Publ. No. 2010/065818; U.S. Pat. No. 8,822,647; PCT Publ. No. WO 2014/031687; U.S. Pat. No. 7,514,537; Brentjens et al., 2007; and Walseng et al., 2017; the techniques of which are herein incorporated by reference.

In certain embodiments, nucleic acid molecules of the present disclosure are used to transfect/transduce a cell, such as a T cell, an NK cell, a macrophage or another immune cell, for use in adoptive transfer therapy. Cells may be induced to incorporate the vector or other material by use of a viral vector, transformation via calcium phosphate precipitation, DEAE-dextran, electroporation, microinjection, or other methods. (Sambrook et al., Molecular Cloning: A Laboratory Manual 2d ed. (Cold Spring Harbor Laboratory, 1989)). T cells and/or NK cells can be collected using known techniques, and the various subpopulations or combinations thereof can be enriched or depleted by known techniques, such as by affinity binding to antibodies, flow cytometry, or immunomagnetic selection. In certain embodiments, the T cell is a CD4+ T cell, a CD8+ T cell, a CD4-CD8-double negative T cell, a naïve T cell, a central memory T cell, an effector memory T cell, a stem cell memory T cell, or any combination thereof. Methods for transfecting/transducing T cells with polynucleotides have been previously described (U.S. Patent Application Pub. No. US 2004/0087025) as have adoptive transfer procedures using T cells of desired target-specificity (Schmitt et al. 2009; Dossett et al. 2009; Till et al. 2008; Wang et al. 2007; Kuball et al., 2007; Leen et al., 2007; U.S. Patent Publ. No. 2011/0243972; U.S. Patent Publ. No. 2011/0189141), such that adaptation of these methodologies to the presently disclosed CARs of the present disclosure is within the scope of the present disclosure.

Functional characterization of CARs described herein may be performed according to any art-accepted methodologies for assaying T cell and/or NK cell activity, including determination of T cell and/or NK cell binding, activation or induction and also including determination of T cell and/or NK cell responses that are antigen-specific. Examples include determination of intracellular calcium, T cell proliferation, T cell and/or NK cell cytokine release, antigen-specific T cell and/or NK cell stimulation, MHC-restricted T cell and/or NK cell stimulation, cytotoxic activity, changes in T cell and/or NK cell phenotypic marker expression, phosphorylation of certain T cell and/or NK cell proteins, and other measures of T cell and/or NK cell functions. Procedures for performing these and similar assays are described herein and/or may be found, for example, in Lefkovits (Immunology Methods Manual: The Comprehensive Sourcebook of Techniques, 1998). See, also, Current Protocols in Immunology, Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein.

### Kits

In some embodiments, kits are provided comprising (a) a CAR vector as disclosed herein, (b) a CAR nucleic acid molecule polynucleotide as disclosed herein, optionally encoding a marker peptide and/or self-cleaving peptide, and/or (c) one or more reagents for transducing the vector or nucleic acid molecule into a cell. In certain embodiments, the kits further comprise instructions for use.

### Methods of Use

The present disclosure also provides methods for treating a disease or condition, wherein the methods comprise administering to a subject in need thereof an effective amount of a composition, cell, or unit dose of the present disclosure, wherein the disease or condition expresses or is otherwise associated with the antigen that is specifically bound by a CAR provided herein. In certain embodiments, the disease or condition is a hyperproliferative or proliferative disease, such as a cancer, an autoimmune disease, or an infectious disease (e.g., viral, bacterial, fungal, or parasitic).

In certain embodiments, a subject to be treated using the methods provided herein is a human. In other embodiments, the subject is a non-human animal, for example in a veterinary or medical research setting. In those embodiments where the subject is a human, the subject can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art.

A composition, cell, or unit dose of the present disclosure may be administered intravenously, intraperitoneally, intratumorally, into the bone marrow, into a lymph node, or into the cerebrospinal fluid so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the disease or condition is a malignancy. In some embodiments, the malignancy is cancer. In general, cancers treatable by presently disclosed methods and compositions include carcinomas, sarcomas, gliomas, lymphomas, leukemias, myelomas, cancers of the head or neck, melanoma, pancreatic cancer, cholangiocarcinoma, hepatocellular cancer, breast cancer, gastric cancer, non-small-cell lung cancer, prostate cancer, esophageal cancer, mesothelioma, small-cell lung cancer, colorectal cancer, glioblastoma, Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma; lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In some embodiments, the cancer is one or more of: prostate cancer, liver cancer, melanoma, leukemia, lymphoma, breast cancer, ovarian cancer, pancreatic cancer, colorectal cancer, lung cancer, bladder cancer, renal cancer, brain cancer, stomach cancer, thyroid cancer, anus cancer, bone cancer, cervix cancer, endometrium cancer, esophagus cancer, eye cancer, gallbladder cancer, thymus, sarcoma and osteosarcoma.

In some embodiments, the cancer comprises a hematologic malignancy.

In some embodiments, the disease or condition is a “hyperproliferative disorder” and “proliferative disorder.” In some embodiments, the hyperproliferative disorders and proliferative disorders is one or more of tumors, cancers, neoplastic tissue, carcinoma, sarcoma, malignant cells, pre malignant cells. In some embodiments, the cancer comprises a solid tumor.

In certain embodiments, the methods provided herein comprise administering a cell expressing a CAR of the present disclosure, a composition comprising the cell, or a unit dose thereof. The amount of cells in a composition is at least one cell (for example, one CAR-modified CD8+ T cell subpopulation; one CAR-modified CD4+ T cell subpopulation; one CAR-modified NK cell subpopulation) or is more typically greater than 10² cells, for example, up to 10⁶, up to 10⁷, up to 10⁸ cells, up to 10⁹ cells, or 10¹⁰ cells or more, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well as the type of cells included therein. For example, cells modified to contain a CAR specific for one or more antigens will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In any of the presently disclosed embodiments, the cell is an allogeneic cell, a syngeneic cell, or an autologous cell.

Also contemplated are pharmaceutical compositions that comprise cells expressing the CARs as disclosed herein and a pharmaceutically acceptable carrier, diluent, and/or excipient. Suitable excipients include water, saline, dextrose, glycerol, or the like and combinations thereof. In embodiments, compositions comprising cells as disclosed herein further comprise a suitable infusion media.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, NK cells) in order to administer a desired, CAR-expressing T-cell composition to a subject as an adoptive immunotherapy. In certain embodiments, the cell is a hematopoietic progenitor cell or a human immune cell. In certain embodiments, the immune system cell is a CD4+ T cell, a CD8+ T cell, a CD4− CD8− double-negative T cell, an NK cell, or any combination thereof. In certain embodiments, the immune system cell is a naïve T cell, a central memory T cell, a stem cell memory T cell, an effector memory T cell, an NK cell, or any combination thereof. In particular embodiments, the cell is a CD4+ T cell. In particular embodiments, the cell is a CD8+ T cell. In particular embodiments, the cell is an NK cell.

As used herein, administration of a composition refers to delivering the same to a subject, regardless of the route or mode of delivery. Administration may be affected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., CAR-expressing recombinant (i.e., engineered) cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a cell as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., cell, CAR, unit dose, or composition) refers to an amount sufficient, at dosages and for periods of time needed, to achieve the desired clinical results or beneficial treatment, as described herein. An effective amount may be delivered in one or more administrations.

Methods according to this disclosure may further include administering one or more additional agents to treat the disease or disorder in a combination therapy. For example, in certain embodiments, a combination therapy comprises administering a CAR (or an engineered cell expressing the same) with (concurrently, simultaneously, or sequentially) an immune checkpoint inhibitor. In some embodiments, a combination therapy comprises administering CAR of the present disclosure (or an engineered cell expressing the same) with an agonist of a stimulatory immune checkpoint agent. In further embodiments, a combination therapy comprises administering a CAR of the present disclosure (or an engineered cell expressing the same) with a secondary therapy, such as chemotherapeutic agent, a radiation therapy, a surgery, an antibody, or any combination thereof.

Cytokines are used to manipulate host immune response towards anticancer activity (see, e.g., Floros & Tarhini, 2015). Cytokines useful for promoting immune anticancer or antitumor response include, for example, IFN-α, IL-2, IL-3, IL-4, IL-5, IL-6, IL-9, IL-10, IL-12, IL-13, IL-15, IL-16, IL-17, IL-17A, IL-17F, IL-18, IL-21, IL-22, IL-24, IFN-γ, TNF-α, and GM-CSF, singly or in any combination with the binding proteins or cells expressing the same of this disclosure.

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times. The headings provided herein are for convenience only and do not interpret the scope or meaning of the described technology.

Various embodiments of the invention are set forth herein below in paragraphs 201 to 253:

A chimeric antigen receptor (CAR) comprising an extracellular region comprising an antigen binding domain, a transmembrane region, and an intracellular region comprising an effector domain and a TMIGD2 costimulatory domain.

The CAR of paragraph 201, wherein the TMIGD2 costimulatory domain comprises the intracellular region of TMIGD2.

The CAR of paragraph 202, wherein the TMIGD2 costimulatory domain comprises a sequence selected from the group consisting of residues 172□282 of SEQ ID NO: 3, 172□278 of SEQ ID NO:4, and residues 52-162 of SEQ ID NO:5.

The CAR of paragraph 203, wherein the TMIGD2 costimulatory domain comprises an amino acid sequence with at least 75% identity to a sequence selected from the group consisting of residues 172□282 of SEQ ID NO:3, 172□278 of SEQ ID NO: 4, and residues 52-162 of SEQ ID NO:5.

The CAR of any one of paragraphs 201-204, wherein the antigen binding domain specifically binds a tumor-associated antigen.

The CAR of paragraph 205, wherein the tumor-associated antigen is selected from the group consisting of HHLA2, CD19; CD20; BCMA; CD22; CD3; CEACAM6; c-Met; EGFR; EGFRvIII; ErbB2; ErbB3; ErbB4; EphA2; IGF1R; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTβR; LIFRβ; LRP5; MUC1; OSMRβ; TCRα; TCRβ; CD25; CD28; CD30; CD33; CD52; CD56; CD79a; CD79b; CD80; CD81; CD86; CD123; CD171; CD276; B7-H3; B7H4; TLR7; TLR9; PTCH1; WT-1; HA1-H; Robo1; α-fetoprotein (AFP); Frizzled; OX40; PRAME; and SSX-2 antigen.

The CAR of any one of paragraphs 201-206, wherein the antigen binding domain comprises an scFv.

The CAR of any one of paragraphs 201-207, wherein the antigen binding domain comprises a linker.

The CAR of paragraph 208, wherein the linker is a glycine-serine linker.

The CAR of paragraph 209, wherein the glycine-serine linker comprises (GlyₓSer_{y})_{z}, wherein x and y are each independently an integer from 0 to 10, provided that x and y are not both 0, and z is an integer from 1 to 10.

The CAR of any one of paragraphs 201-210, wherein the extracellular region further comprises an N-terminal leader sequence.

The CAR of any one of paragraphs 201-211, wherein the extracellular region further comprises a hinge region.

The CAR of paragraph 212, wherein the hinge region comprises the amino acid sequence set forth in SEQ ID NO:2.

The CAR of paragraph 213, wherein the hinge region comprises an amino acid sequence with at least 75% identity to the amino acid sequence set forth in SEQ ID NO: 2.

The CAR of any one of paragraphs 201-214, wherein the transmembrane region comprises a CD8α transmembrane region.

The CAR of paragraph 215, wherein the transmembrane region comprises the amino acid sequence set forth in SEQ ID NO:1.

The CAR of paragraph 215, wherein the transmembrane region comprises an amino acid sequence with at least 75% identity to the amino acid sequence set forth in SEQ ID NO:1.

The CAR of any one of paragraphs 201-217, wherein the effector domain is a CD3ζ effector domain.

The CAR of paragraph 218, wherein the effector domain comprises the amino acid sequence set forth in SEQ ID NO:6.

The CAR of paragraph 218, wherein the effector domain comprises an amino acid sequence with at least 75% identity to the amino acid sequence set forth in SEQ ID NO: 5.

The CAR of any one of paragraphs 201-220, wherein the CAR comprises (a) a sequence selected from the group consisting of residues 172□282 of SEQ ID NO:3, 172□278 of SEQ ID NO:4, and residues 52-162 of SEQ ID NO:5; and (b) the sequence set forth in SEQ ID NO:1.

The CAR of any one of paragraphs 201-220, wherein the CAR comprises (a) a sequence selected from the group consisting of residues 172□282 of SEQ ID NO:3, 172□278 of SEQ ID NO:4, and residues 52-162 of SEQ ID NO:5; and (b) the sequence set forth in SEQ ID NO:6.

The CAR of any one of paragraphs 201-220, wherein the CAR comprises (a) a sequence selected from the group consisting of residues 172□282 of SEQ ID NO:3, 172□278 of SEQ ID NO:4, and residues 52-162 of SEQ ID NO:5; and (b) the sequence set forth in SEQ ID NO:1; and (c) the sequence set forth in SEQ ID NO:6.

The CAR of paragraph 222 or 223, wherein the CAR further comprises the sequence set forth in SEQ ID NO:2.

An isolated nucleic acid molecule comprising a nucleic acid sequence encoding the CAR of any one of paragraphs 201-223.

A vector comprising a nucleic acid sequence encoding the CAR of any one of paragraphs 201-223.

The vector of paragraph 226, wherein the nucleic acid sequence encoding the CAR is operably linked to an expression control sequence.

The vector of paragraph 227, wherein the expression control sequence is a promoter.

The vector of any one of paragraphs 226-228, further comprising a nucleic acid sequence encoding a self-cleaving peptide.

The vector of paragraph 229, wherein the self-cleaving peptide is a 2A self-cleaving peptide.

The vector of paragraph 230, wherein the 2A self-cleaving peptide is a P2A peptide.

The vector of any one of paragraphs 226-231, further comprising a nucleic acid sequence encoding a transduction marker polypeptide.

The vector of paragraph 232, wherein the transduction marker polypeptide is a truncated form of epidermal growth factor receptor (EGFRt) or GFP, or a portion or variant thereof.

The vector of any one of paragraphs 230-233, wherein the nucleic acid sequence encoding the self-cleaving peptide is 3′ of the nucleic acid sequence encoding the CAR.

The vector of paragraph 233 or 234, wherein the vector comprises a nucleic acid sequence encoding a self-cleaving peptide, and wherein the nucleic acid sequence encoding the self-cleaving peptide is 5′ of the nucleic acid sequence encoding the marker polypeptide.

The vector of any one of paragraphs 226-235, wherein the vector is a viral vector.

An isolated cell expressing the CAR of any one of paragraphs 201-225.

The cell of paragraph 237, wherein the cell comprises the nucleic acid molecule of paragraph 218.

The cell of paragraphs 237 or 238, wherein the cell comprises a vector of any one of paragraphs 225-234.

The cell of any one of paragraphs 237-239, wherein the cell is a T cell, a natural killer (NK) cell, a macrophage, or other immune cell.

The cell of paragraph 240, wherein the T cell is a CD4+ T cell, a CD8+ T cell, a CD4-CD8-double negative T cell, an NK cell, a macrophage, other immune cell, or any combination thereof.

The cell of paragraph 240, wherein the T cell is a naïve T cell, a central memory T cell, a stem cell memory T cell, an effector memory T cell, an NK cell, a macrophage, other immune cell, or any combination thereof.

The cell of any one of paragraphs 237-242, wherein the cell further expresses a transduction marker on its surface.

The cell of paragraph 243, wherein the transduction marker is a truncated form of epidermal growth factor receptor (EGFRt) or GFP, or a portion or variant thereof.

A method of treating a disease or condition in a subject in need thereof comprising administering to the subject an effective amount of the cell of any one of paragraphs 237-244.

The method of paragraph 245, wherein the disease or condition is a malignancy.

The method of paragraph 246, wherein the malignancy is a cancer.

The method of paragraph 247, wherein the cancer is selected from the group consisting of prostate cancer, liver cancer, melanoma, leukemia, lymphoma, breast cancer, ovarian cancer, pancreatic cancer, colorectal cancer, lung cancer, bladder cancer, renal cancer, brain cancer, stomach cancer, small intestine cancer, bone cancer, cervix cancer, endometrium cancer, eye cancer, gallbladder cancer, thyroid cancer, thymus cancer, sarcoma, and osteosarcoma.

The method of paragraphs 247 or 248, wherein the cancer comprises a solid tumor.

The method of any one of paragraphs 247-249, wherein the cancer comprises a hematologic malignancy.

A method of eliciting an immune response against a tumor-associated antigen that specifically binds the CAR of any one of paragraphs 201-225 in a subject, comprising administering to the subject an effective amount of the cell of any one of paragraphs 237-244.

A composition comprising a CAR of any one of paragraphs 201-225 and a pharmaceutically acceptable excipient, carrier, or diluent.

A composition comprising a cell of any one of paragraphs 237-244 and a pharmaceutically acceptable excipient, carrier, or diluent.

Any patents, applications and other references cited herein are incorporated herein by reference. Aspects of the described technology can be modified, if necessary, to employ the systems, functions, and concepts of the various references described above to provide yet further embodiments.

These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

The foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

The following example is illustrative of several embodiments of the present technology:

### EXAMPLES

### Example 1: CAR-T Cell Therapy Using CAR-T Cells Comprising TMIGD2 as a Costimulatory Molecule

The following example demonstrates the generation of a new CAR-T vector containing the cytoplasmic tail of TMIGD2 (transmembrane and immunoglobulin domain containing 2), wherein the CAR-T vector is capable of transducing normal T cells and can kill human tumor cells.

Generation of a CAR-T Vector with TMIGD2

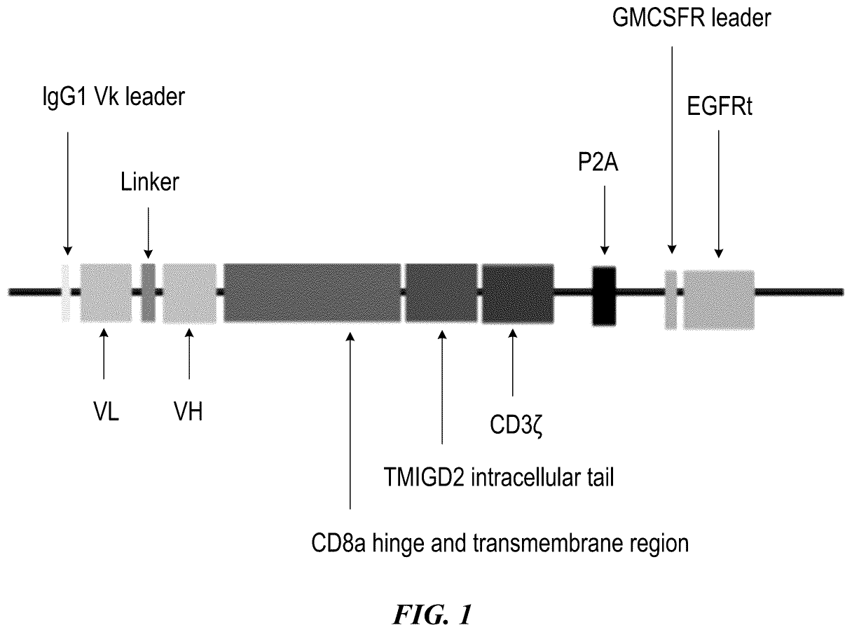

A CAR-T vector using TMIGD2 as a costimulatory molecule has been developed (FIG. 1). The new vector contains an antibody signal leader, VH, VL, human CD8α hinge and transmembrane region, human TMIGD2 intracellular tail, human CD3ζ, P2A, human GMCSFR signal leader, and human EGFRt.

### TGMID2 CAR-T Mediated Killing of Human Tumor Cells

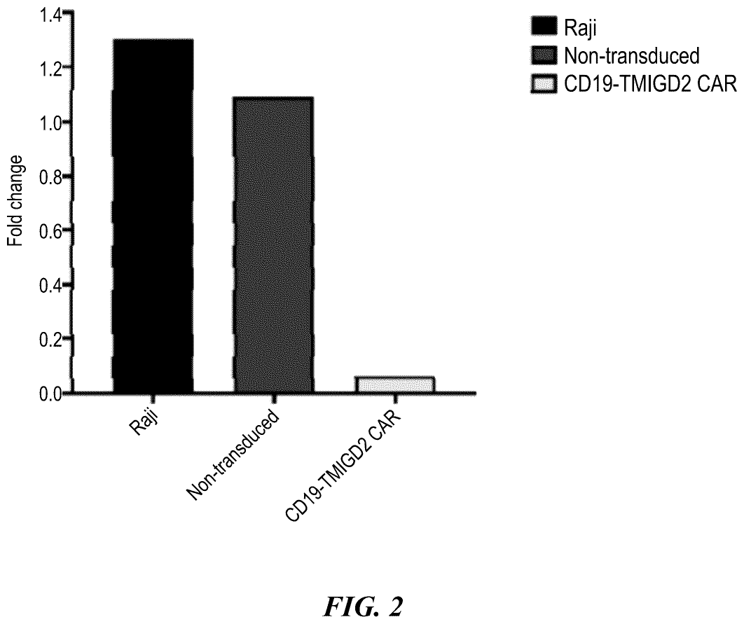

As a proof of principle, VH and VL of a mAb against human CD19 were cloned into the TMIGD2 vector shown in FIG. 1 and experiments for CAR-T mediated killing of human tumor cells were performed. T cells from normal PBMCs were transfected with the TMIGD2 vector to generate CAR-T cells. Human Raji tumor cells expressing the CD19 antigen were then incubated alone, with non-transduced T cells, and with CAR-T transduced T cells. The results showed that CAR-T cells, but not non-transduced T cells, killed almost all of the Raji tumor cells (FIG. 2), demonstrating that the CAR-T cells including a TMIGD2 costimulatory domain are able to efficiently kill tumor cells.

### In Vivo Therapeutic Efficacy of CD19-TMIGD2 CAR-T Cells

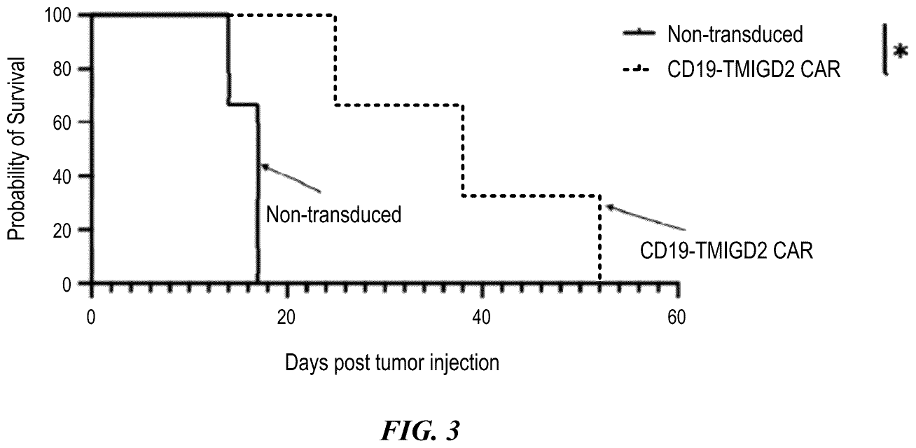

This example demonstrates the in vivo therapeutic efficacy of CD19-TMIGD2 CAR-T cells in a Raji lymphoma model. NSG™ mice were intravenously (I.V.) injected with 0.25×10\hat{ }6 Raji tumor cells on day 0, and with 10\hat{ }7 non-transduced T cells or CD19-TMIGD2 CAR-T cells on day 3 and 10, respectively. The mice were monitored for survival. As demonstrated in FIG. 3, the survival of the mice received CD19-TMIGD2 CAR-T cells was significantly improved compared to the non-transduced control mice.

### Example 2: CAR-T Cell Therapy Using Anti B7-H3 CAR-T Cells Comprising TMIGD2 as a Costimulatory Molecule

The following example demonstrates the generation of anti-B7-H3 CAR-T cells, which incorporate a vector containing the cytoplasmic tail of TMIGD2 (transmembrane and immunoglobulin domain containing 2), wherein the CAR-T vector transduces normal T cells and kills human tumor cells expressing B7-H3 in vitro and in vivo.

### Experimental Methods

### Cell Lines

NIH 3T3 cells (mouse fibroblast) were obtained from the American Type Culture Collection (ATCC); NSO cells (mouse multiple myeloma) from Department of Cell Biology, Albert Einstein College of Medicine, HEK293T cells (human epithelial kidney) from Department of Cell Biology, Albert Einstein College of Medicine, U118 cells (human glioblastoma) from the ATCC, HCC827 cell (human lung adenocarcinoma) from the ATCC, THP-1 cells (human acute monocytic leukemia) from the ATCC, Raji cells (B cell Burkitts lymphoma) from the ATCC, Jurkat (NFAT) cells (T cell leukemia) from BPS Bioscience, AsPC-1 cells (pancreatic adenocarcinoma) from the ATCC, PANC-1 cells (pancreatic ductal epitheloid carcinoma) from the ATCC, and Phoenix-AMPHO cells (human epithelial kidney) from the ATCC. Cell lines were grown in DMEM or RPMI 1640 media supplemented with 10% FBS, 1% penicillin, and 1% streptomycin and cultured in a humidified incubator at 37° C. and 5% CO2.

### Transfection of Tumor Cell Lines

Phoenix-AMPHO cells were co-transfected with a MSCV-YFP plasmid containing the protein-of-interest (mouse B7-H3, human B7-H3, cynomolgus B7-H3, or Luciferase-tdTomato (−Luc)) and the pCMV-VSV-G plasmid. 48 and 72 hour viral supernatant was collected and used to transfect tumor cell lines using polybrene. Transfected cells were sorted at least twice to ensure pure populations using a BD FACS Aria II cell sorter.

### Generation of Anti-B7-H3 Monoclonal Antibodies

C57BL/6 mice were immunized using a recombinant protein comprised of the IgV domain of human B7-H3 fused to a human Fc fragment. After immunization, hybridomas were generated by fusing NSO myeloma cells and mouse splenocytes using standard methods. Antibody-producing hybridomas were screened by flow cytometry to ensure specific binding to mouse and human B7-H3. Afterwards, hybridomas were thrice subcloned by single cell dilution and then expanded in the cell compartment of bioreactor flasks in DMEM high glucose media (supplemented with 10% ultra-low IgG FBS, 10% NCTC-109, 1% penicillin, 1% streptomycin, and 1% non-essential amino acids. The media compartment of the bioreactor flasks contained DMEM high glucose media supplemented with 1% penicillin and 1% streptomycin. Antibody supernatant from the cell compartment was collected and stored at 4° C. until purification using Protein G resin-packed columns. Purified B7-H3 mAbs were analyzed using SDS-PAGE prior to affinity determination, isotype determination, and VH and VL sequencing.

### B7-H3 mAb Affinity Determination

Anti-B7-H3 mAb affinity to mouse and human B7-H3 was determined by biolayer interferometry. Recombinant mouse and human B7-H3-Fc proteins were loaded onto mouse or human capture biosensors, respectively. Afterwards, the protein-loaded biosensors were placed into solutions containing serial dilutions of anti-B7-H3 mAbs. Kon, Koff, and KD were determined by analysis using a 1:1 binding model.

### Generation of CAR Constructs

Anti-B7-H3 single chain variable fragments (scFvs) were generated by cloning the native mAb signal peptide sequence to the VH and VL regions of anti-B7-H3 mAbs connected by a G4S linker. The scFv was connected to a human CD8α hinge and transmembrane domain, the intracellular region of various costimulatory proteins (CD28, 4-1BB, TMIGD2, CD28-4-1BB, or TMIGD2-4-1BB), followed by the intracellular domain of human CD3ζ. A self-cleaving P2A peptide sequence was then inserted followed by the signal peptide from the granulocyte-macrophage colony-stimulating factor receptor (GM-CSFR)-α chain and a truncated human epidermal growth factor receptor (hEGFRt) protein for CAR detection. For in vivo CAR-T cell persistence experiments, the hEGFRt protein was followed by a self-cleaving T2A peptide sequence, followed by a firefly luciferase gene sequence. The entire CAR sequence was cloned into the pL VX-Zsgreen lentiviral expression plasmid under the control of an EF1α promoter. A commercially available CD19-CD28-4-1BB CAR was also modified to include the P2A-EGFRt and T2A-Luciferase sequences.

Production and Determination of Viral Titer from CAR Lentivirus

HEK293T cells were co-transfected with the psPAX packaging plasmid, pMD2.G envelope plasmid, and CAR plasmid. 48- and 72-hour viral supernatant was collected, concentrated 100×, and subsequently pooled to ensure equivalent titer. Virus titer was determined by transducing activated human T cells with the CAR lentivirus as described below. Viral titer was calculated based on EGFR+ T cells.

### Isolation of Human T Cells

Leukopaks from healthy human donors were obtained and peripheral blood mononuclear cells (PBMCs) were isolated using density gradient centrifugation with Lymphoprep. T cells were purified by negative enrichment. T cells were either frozen in freezing media and stored in liquid nitrogen or were used immediately.

### Generation of CAR-T Cells

Fresh or thawed T cells were activated for 24 hours on an OKT3 (1 ug/mL) and CD28 (1 μg/mL) antibody-coated 24-well plate in CTS OpTmizer media supplemented with OpTmizer T-cell expansion supplement, 10% FBS, 1% L-glutamine, 1% penicillin, 1% streptomycin, IL-7, and IL-15. Activated T cells were then transduced on non-tissue culture treated plates coated with RetroNectin reagent (19 ug/mL) and CAR lentivirus (MOI of approximately 10). CAR-T cells were then expanded for at least 7 days before use in experiments. Prior to all experiments, CAR-T cell transduction efficiency was normalized to 50% CAR+ cells (for in vitro cytotoxicity screening assay) or to the lowest efficiency donor by adding of non-transduced T cells. If necessary, CAR-T cells were purified using anti-phycoerythrin (PE) microbeads following anti-EGFR-PE staining.

### In Vitro Coculture Killing Assays

0.1×10⁶ CAR-T cells and either HCC827-Luc (0.01×10⁶ cells), U118-Luc, (0.01×10⁶ cells), and THP-1 (0.01×10⁶ cells) were plated in T cell media without the addition of cytokines. 3-5 days later, the tumor cells were enumerated with flow cytometry. For the Incucyte time-lapse cytotoxicity assays, 5×10³ HCC827-Luc or U118-Luc cells were plated one day prior to imaging. Tumor-coated plates were then placed in the Incucyte machine and imaged every 4 hours at 4 locations per well for a total of 112 hours. 16 hours after the initial imaging, 0.1×10⁶ CAR-T cells were gently added to the wells. Imaging data was analyzed using the Incucyte software.

### Flow Cytometry

Cells were stained using antibodies conjugated to the following fluorophores: fluorescein isothiocyanate (FITC), phycoerythrin (PE), allophycocyanin (APC), APC-Fire 750, PE-cyanine (Cy)7, peridinin-chlorophyll-protein (PerCP)-Cy5.5, brilliant violet (BV)-421, BV-711, alexa fluor (AF)-532, AF-488, brilliant ultra violet (BUV)-496, BUV-395, BUV-496, and BUV-737. Expression of tdTomato and YFP was also used to distinguish cell populations. Antibody targets include CD45, CD3, CD4, CD8, CD45RA, CCR7, EGFR, G4S linker, PD-1, TIM-3, LAG-3, B7-H3, CD69, and CD33. Viability was determined using 7-amino-actinomycin D (7-AAD), zombie NIR, ghost violet 510, and 4′,6-diamidino-2-phenylindole (DAPI). In some experiments, cells were fixed using a 2% paraformaldehyde (PFA) solution prior to analysis. All samples were acquired using a BD LSRII or Cytek Aurora flow cytometer. Data analysis was performed on FlowJo (or SpectroFlo. T-distributed stochastic neighbor embedding (t-SNE) plots were generated using the t-SNE plugin on FlowJo.

### Flow Cytometry Cytokine Analysis

0.4×10⁶ CAR-T cells and 0.1×10⁶ HCC827 tumor cells were cocultured for 24 hours in 24-well plates. Supernatants from the cocultures were collected and stored at −80° C. until analysis. The concentration of IL-2, IL-4, IL-5, IL-6, IL-9, IL-10, IL-13, IL-17A, IL-17F, IL-22, IFN-γ, and TNF-α was determined using the flow cytometry-based LEGENDplex human Th cytokine panel kit on non-diluted or 1:5 diluted supernatant samples. Data was analyzed using the LEGENDplex online data analysis software suite.

### RNA Isolation

For in vitro RNA isolation, CAR-T cells were gently removed from coculture wells and plated into fresh wells for 30 minutes to allow excess tumor cells to attach to the plate. CAR-T cells were gently removed and live cells enriched using Lymphoprep density gradient centrifugation. T cells were further purified isolated using a CD3+ selection beads. RNA was extracted and stored at −80° C. RNA sequencing (40 million paired-end reads) after library preparation (using the NEBNext Ultra II kit) was performed by Admera Health. For in vivo RNA isolation, lung-infiltrating T cells were isolated from mouse lungs. Briefly, single cell suspensions were prepared from lungs by enzymatic digestion (Collagenase IV (200 IU·mL), dispase (0.5 IU/mL), and DNase I (100 U/mL) in RPMI 1640 media. Next, the single cell suspension was layered on a discontinuous density gradient Percoll solutions (40% and 80%), and the immune cells at the 40% 80% interface were collected. A second density gradient centrifugation using Lymphoprep was performed to further enrich the live cell population. Mouse cells were then depleted two times. Afterwards, T cells from the same donor transduced with the same CAR were pooled, and RNA was isolated as described above. RNA sequencing (40 million paired-end reads) after library preparation (using the SMARTseq V4 with NExtera XT kit) was performed by Admera Health

### B7-H3 CAR Vs CD19 CAR RNA Sequencing Analysis

Reads from RNA sequencing were aligned using STAR alignment software (version 2.6.1b) to a reference human genome (hg38; downloaded in November 2019 from the UCSC genome browser). Gene-mapped fragments were counted using HTseq software (version 0.6.1). Genes with an average expression count of 1 or more in either group were considered expressed. Differentially expressed genes (DEGs) were determined by an adjusted p value of <0.05 and log 2 fold change >0.5. Principle component analysis and differential expression analysis were then performed using DESeq2 software (version 3.10). Gene set enrichment analysis (GSEA) was performed using the Hallmark, Kegg, and Reactome databases using ranked gene lists determined by multiplying the −log 10 p-value and the sign of the log 2 (fold change).

### B7-H3 CAR In Vitro and In Vivo RNA Sequencing Analysis

Paired-end reads from RNA sequencing were aligned using the STAR aligner (version 2.7.9a) to the human reference genome (hg38). The alignment data were then used by the RSEM software (version 1.3.3) to quantify expression levels of individual genes (both coding and non-coding) in the GENCODE annotation (version 41), yielding estimated read counts and transcripts per million (TPMs). Genes with a TPM<1 in all samples were excluded from further analysis. Differentially expressed genes (DEGs) were determined by an adjusted p value of <0.05 and log 2 fold change >0.5. Principal component analysis (PCA) and differential expression analysis were then performed using the DESeq2 software (version 1.38.3). Gene Set Enrichment Analysis (GSEA) was performed using the Hallmark, KEGG, and Reactome gene sets after ranking genes by multiplying the −log 10 (p-value) and the sign of log 2 (fold change) for each gene. Gene sets with a false discovery rate (FDR)<0.05 were considered enriched. Over representation analysis was also performed on DEGs or significantly differentially expressed genes (adjusted p-value <0.05) using the cluster. Profiler (version 4.6.2) enrichGO function. Pathways with an adjusted p value <0.05 were considered enriched in either the up- or down-regulated genes. Gene sets describing T cell dysfunction and metabolism were obtained from the literature, and the inhibitory protein list was manually curated.

### Seahorse Metabolic Assay

0.1×10⁶ CAR-T cells were cocultured alone or with 5×10³ HCC827 tumor cells for one or seven days. CAR-T cells were collected from the wells and transferred to a new well for 30 minutes to allow for attachment of any tumor cells. CAR-T cells were re-collected and analyzed using the Seahorse T cell metabolic profiling kit on Seahorse PDL-coated plates. After transfer to PDL-coated plates, CAR-T cells were rested 5 minutes before centrifugation to allow for even cell distribution on the bottom of the well. Seahorse plates were run on a Seahorse XF96 analyzer at baseline and after injections of Oligomycin A, Bam15, and Rotenone/Antimycin A. Data was analyzed using Agilent Seahorse Analytics online software. % ATP from mitochondria was obtained by the following formula: 100%-% ATP from glycolysis.

### Jurkat (NFAT) T Cell Activation Experiment

Jurkat (NFAT) cells were transduced with CAR constructs as described above. Triplicate wells in a 96-well plate were coated with either OKT3 (1 μg/mL) or 0.2×10⁵ tumor cells (U118, HCC827, or AsPC-1) overnight. 0.2×10⁵ Raji tumor cells were added to additional triplicate wells. 0.1×10⁶ non-transduced or CAR-transduced Jurkat (NFAT) cells were added to each well and incubated for 6 hours. The Bio-Glo Luciferase assay system was used to detect the Jurkat (NFAT) luciferase. Luciferase signal was acquired on a plate reader.

### Chronic Antigen Exposure

0.2×10⁶ CAR-T cells were plated in 96-well plates in triplicate wells using T cell media without cytokines. 0.1×10⁵ HCC827 tumor cells were added to each well. Every 3-4 days, CAR-T cells were gently removed from the well and transferred to a new well. A small cell aliquot was then analyzed using flow cytometry. Unanalyzed CAR-T cells were centrifuged, and additional supernatant was removed such that one half of the original volume remained in the well. 0.1×10⁵ HCC827 parental cells in fresh T cell media were then added at an equivalent volume. This was repeated for 17 days.

### Mice

For all in vivo experiments, 8-12 week old female NOD.Cg-Prkdcscid Il2rgtm1Wjl/SzJ (NSG) mice were used. NSG mice were purchased from The Jackson Laboratory and bred at the Albert Einstein College of Medicine. Mice were housed in a specific-pathogen free animal facility under a 12-hour light/dark cycle with food and water freely available. All experimental procedures were approved by the Albert Einstein Institutional Animal Care and Use Committee.

### Bioluminescent Imaging

Bioluminescent imaging was performed using the IVIS Spectrum in vivo imaging system and analyzed using Living Image software (version 3.0). Images were acquired 10-15 minutes after intraperitoneal (I.P.) injection of D-Luciferin (150 μg/g of mouse weight).

### In Vivo Lung Cancer Model

0.5×10⁶ HCC827-Luc cells were injected I.V. in the lateral tail vein of NSG mice in 100 μL of sterile PBS. Three days later, tumor engraftment was confirmed via IVIS imaging and experimental groups were normalized to ensure equivalent baseline tumor burden. One injection of 10×10⁶ CAR-T cells was then given I.V. into the lateral tail vein, followed by an additional injection one week later. Tumor burden was tracked via IVIS imaging over the course of 100 days. Survival was also tracked during this time.

### In Vivo Glioblastoma Model

U118-Luc tumor cells were resuspended at 5×10³ cells/μL in sterile PBS. A small hole was drilled in the skull using an 18-gauge needle 1 mm lateral and 1 mm anterior to bregma using the freehand method in NSG mice anesthetized with continuous isoflurane (2%). 2 μL of tumor cells were injected into the hole perpendicular to the benchtop surface using a blunt end Hamilton syringe fitted with a sterile pre-cut pipette tip which exposed 1 mm of the syringe needle over the course of one minute. Meloxicam (5 mg/kg) was administered pre-emptively and for two days after tumor implantation. 7 days later, tumor engraftment was confirmed using IVIS imaging. Mice were then allocated into experimental groups normalized for baseline tumor burden. Afterwards, 1×10⁶ CAR-T cells were injected intratumorally (I.T.) using the same surgical preparation described above in a 2 μL volume of PBS. Tumor burden and survival was tracked via IVIS imaging for 100 days.

### In Vivo Pancreatic Cancer Model

PANC-1-Luc tumor cells were resuspended at 1×10⁴ cells/50 μL of sterile PBS. The pancreas was expressed though a 1 cm incision made in the skin and musculature of NSG mice. 50 μL of tumor cells were injected into the head of the pancreas using a 27 gauge needle. Meloxicam was administered pre-emptively and for three days after tumor implantation. Seven days later, tumor engraftment was confirmed using IVIS imaging. Mice were then allocated into experimental groups normalized for baseline tumor burden. 10×10⁶ CAR-T cells were then injected I.V. into the lateral tail vein, followed by another injection 7 days later. Tumor burden and survival was tracked via IVIS imaging for 100 days.

### In Vivo CAR-T Cell Persistence Model

0.5×10⁶ parental HCC827 tumor cells were I.V. injected into the lateral tail vein. Three days later, a single dose of 10×10⁶ CAR-Luc T cells was injected I.V. into the lateral tail vein. CAR-Luciferase T cell signal was tracked using IVIS imaging over the course of 46 days.

Immune Cell Isolation from Mouse Blood and Organs

To obtain immune cells from mouse blood, blood was taken from the lateral tail vein in heparinized capillary tubes. Red blood cells (RBCs) were then lyzed. Remaining cells were then washed twice with PBS before flow cytometry analysis. Mice were euthanized and organs of interest were directly removed (spleens) or perfused with PBS then removed (lungs). Spleens were then transferred to C tubes and mechanically dissociated. The cell suspension was strained through a 40 μM filter. To obtain immune cells from the lungs, single cell suspensions were made using enzymatic digestion, and immune cells isolated using discontinuous Percoll gradients. RBC lysis buffer was performed to remove RBCs.

### Statistics

Statistical analysis was performed using GraphPad prism software (version 9.4.1). As described in each figure legend, testing between groups was performed using an unpaired two-tailed Student's t-test, one-way ANVOA, Kruskal-Wallis test, two-way ANOVA, or Mantel-Cox log-rank test. The data are shown as individual values, or as individual values and the mean±SEM. A P value of less than 0.05 was considered statistically significant.

### Generation and Validation of Anti-B7-H3 Monoclonal Antibodies

Six anti-B7-H3 mAb clones denoted as 1G5, 15F9, 23B2, 8B12, 12B4, and 24D12 that bound to both mouse and human B7-H3 at affinities ranging from 0.32 nM to 11.08 nM using biolayer interferometry (BLI) were obtained. All of the anti-B7-H3 mAbs bound to mouse and human B7-H3 were validated that each stably expressed on 3T3 cells by flow cytometry. Additionally, each anti-B7-H3 mAbs was tested to confirm that each could bind to non-human primate cynomolgus B7-H3. All anti-B7-H3 mAbs were able to detect cynomolgus B7-H3 stably expressed on 3T3 cells by flow cytometry. The anti-B7-H3 mAbs were able to recognize human, mouse, and cynomolgus B7-H3.

### In Vitro Screening of Anti-B7-H3 CAR-T Cells

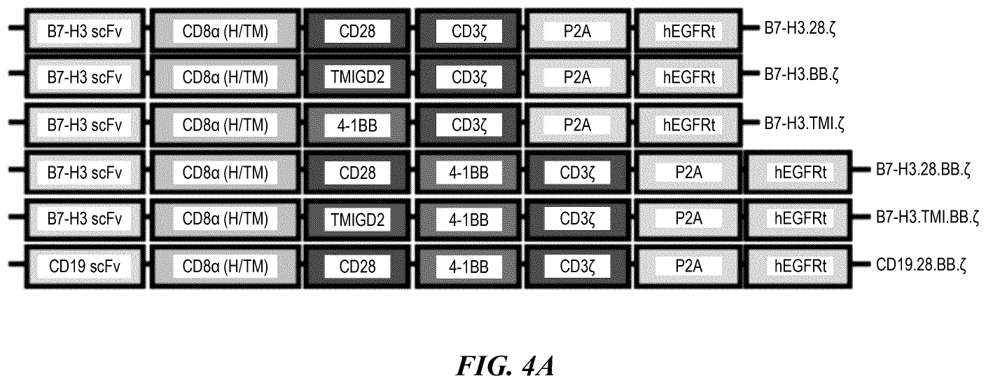

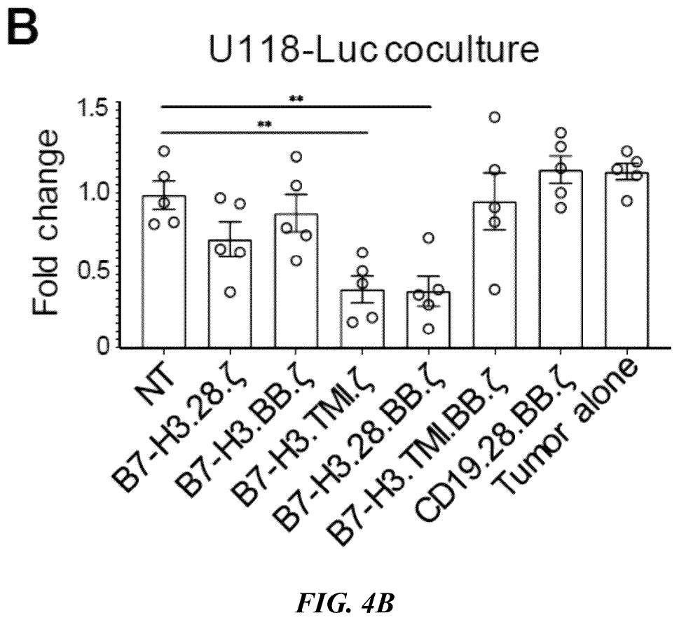

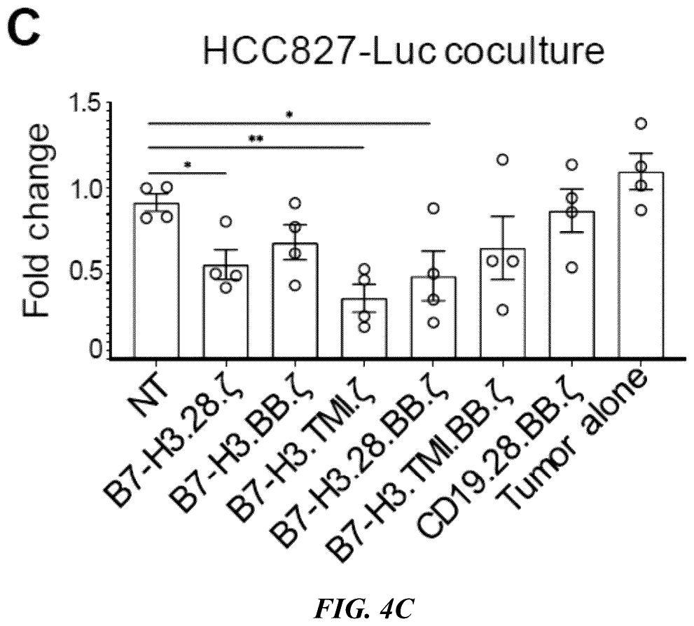

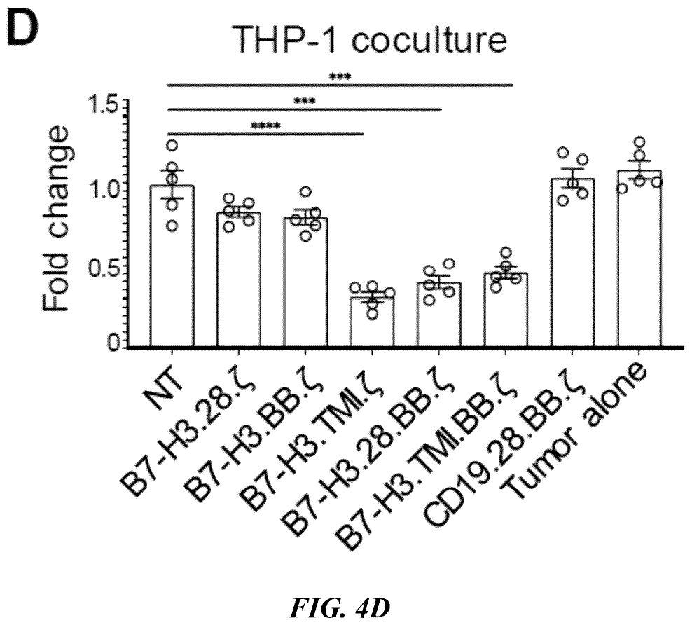

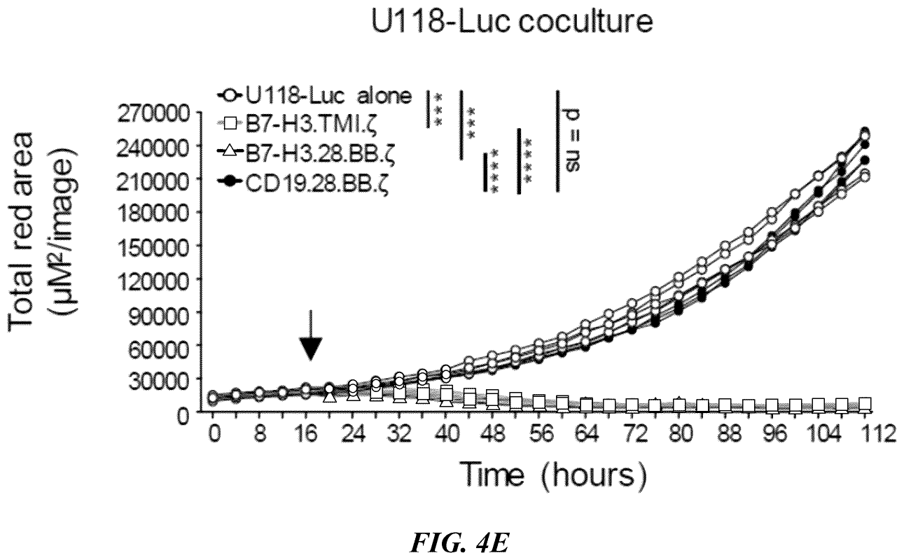

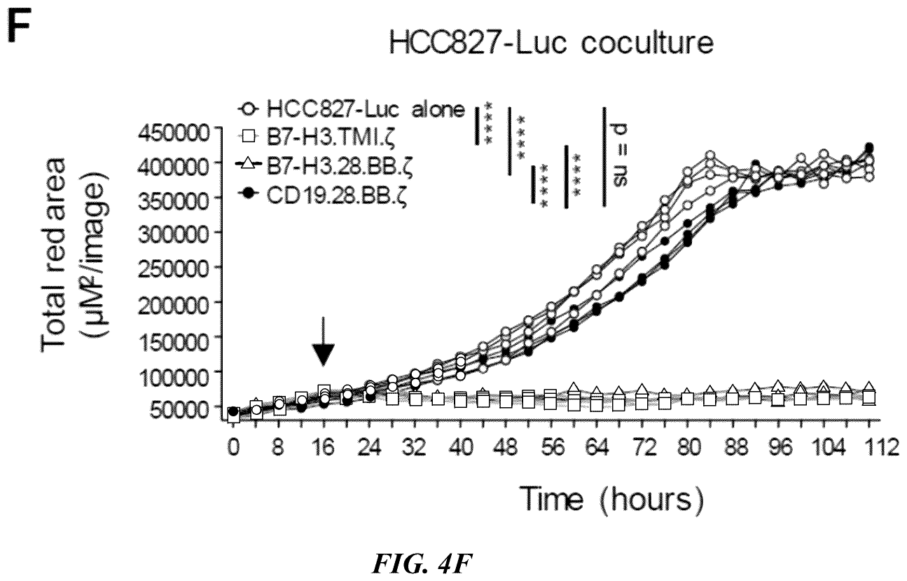

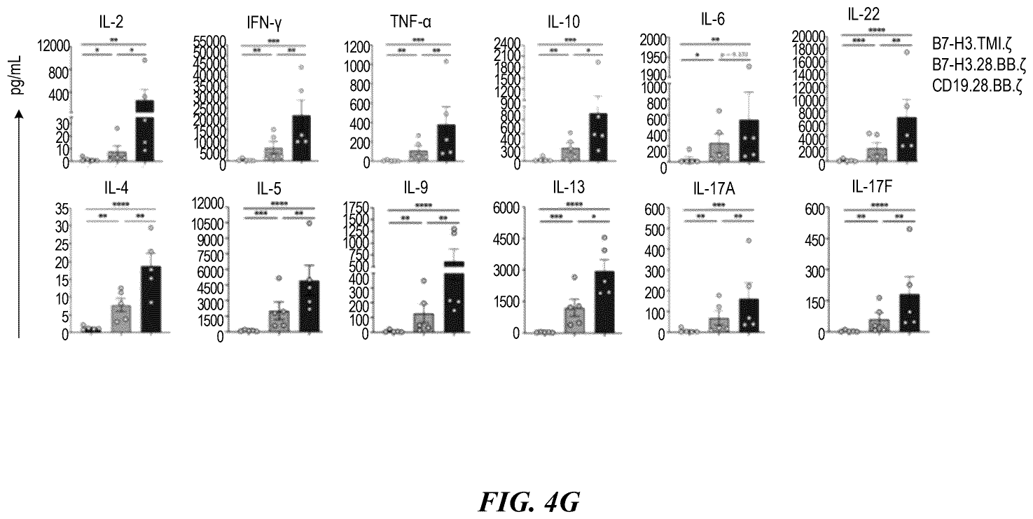

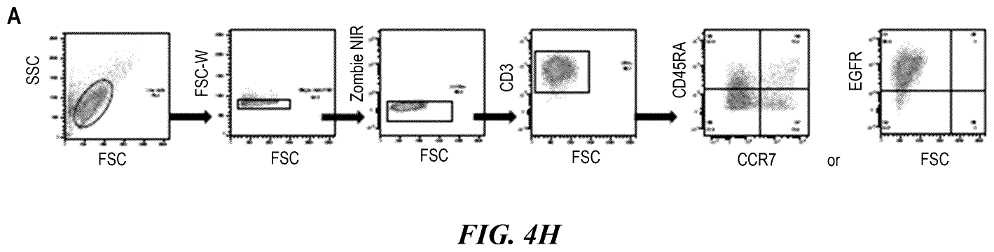

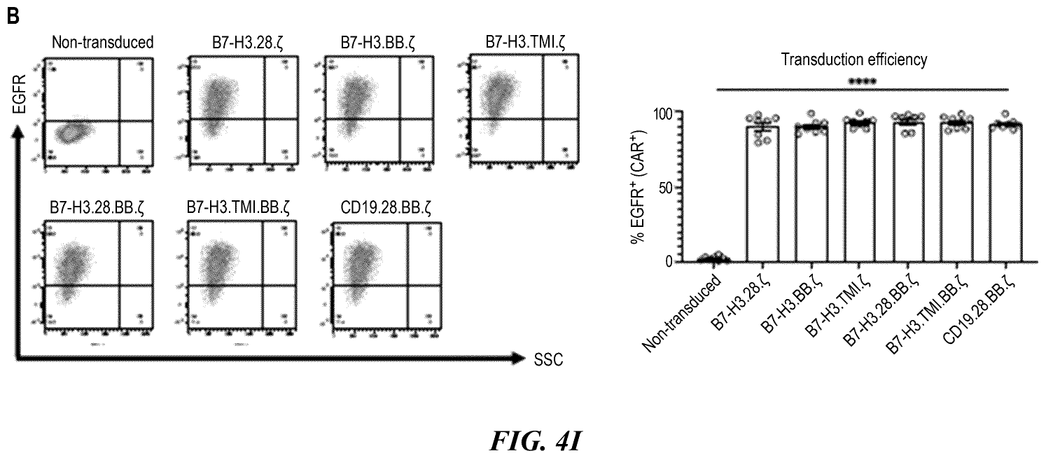

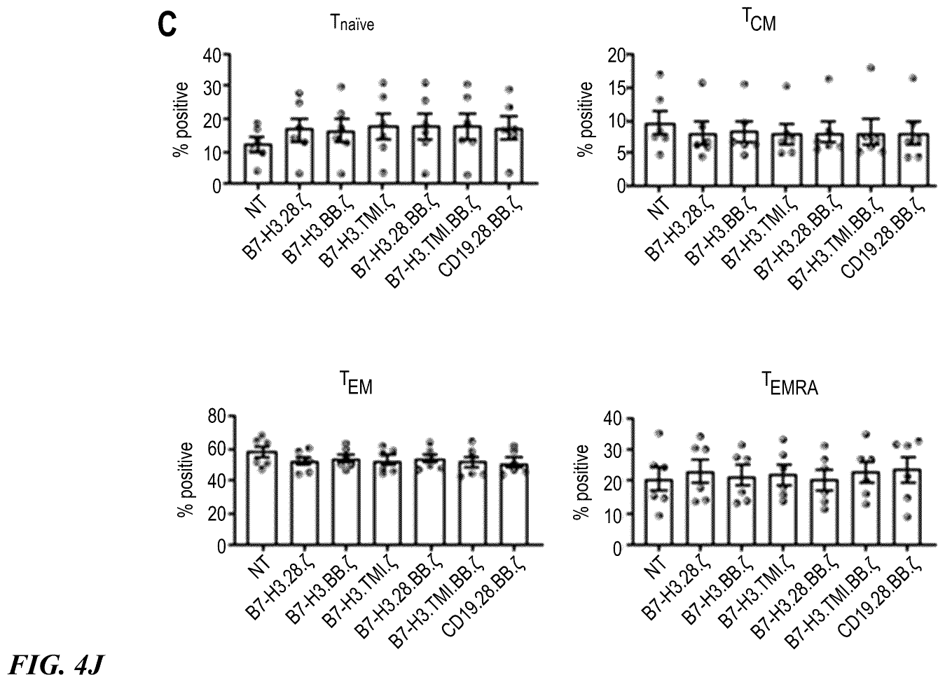

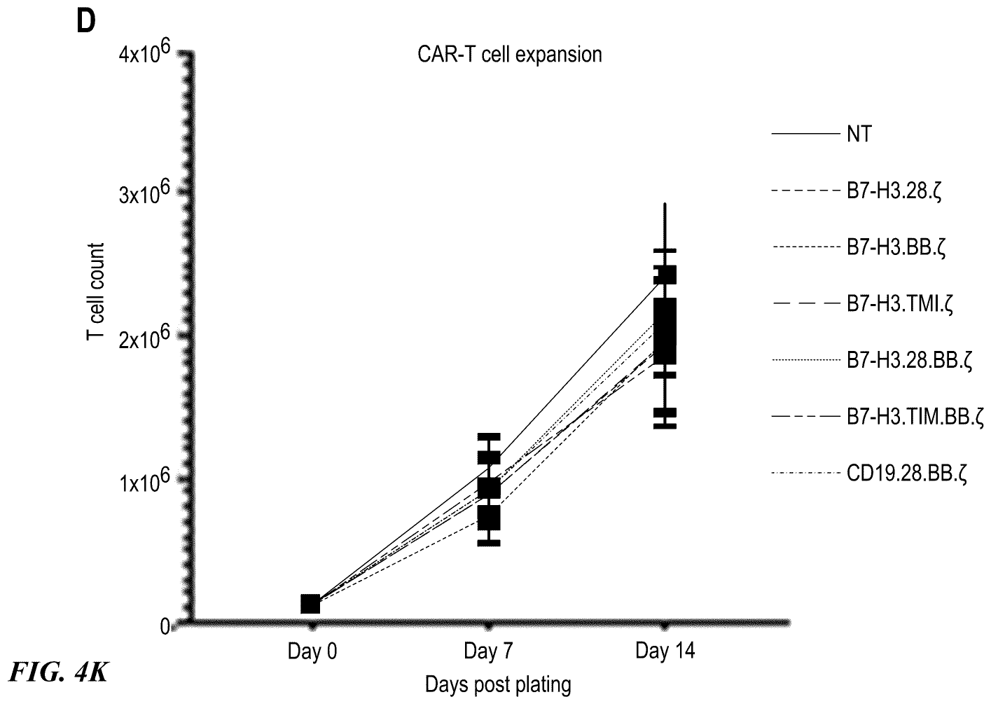

Using a single chain variable fragment (scFv) derived from anti-B7-H3 mAb clone 8B12, anti-B7-H3 CARs were constructed by sequentially linking the scFv to a human CD8α hinge and transmembrane (H/TM) domain, the intracellular domain of various costimulatory proteins, and the intracellular domain of CD3ζ. After the CD3ζ sequence, a self-cleaving P2A peptide and a truncated human epidermal growth factor receptor (hEGFRt) were included (FIG. 4A). The hEGFRt protein was modified by removing two of four extracellular domains and all intracellular domains to prevent antigen binding and signaling. As it can still be recognized by anti-EGFR antibodies, it functioned as marker of transduction efficiency. The following costimulatory domains were utilized in the CAR constructs: CD28 (B7-H3.28.ζ CAR), 4-1BB (B7-H3.BB.ζ CAR), TMIGD2 (B7-H3.TMI.ζ CAR), CD28-4-1BB (B7-H3.28.BB.ζ CAR), or TMIGD2-4-1BB (B7-H3.TMI.BB.ζ CAR). An anti-CD19 CAR was similarly generated using an anti-CD19 scFv and with the CD28-4-1BB costimulatory domains to serve as an irrelevant target control (CD19.28.BB.ζ) (FIG. 4A). On primary human T cells, all CAR constructs were efficiently expressed with transduction efficiencies regularly greater than 85% CAR+ as determined by expression of the hEGFRt protein, and there was no difference in memory phenotype or expansion between the CAR-T cell constructs (FIG. 4H-K).

The in vitro anti-tumor response of the B7-H3 CAR-T cells was tested against multiple tumor types which were B7-H3+ by flow cytometry. The CAR-T cell transduction efficiency was lowered to 50% using non-transduced T cells to better reflect transduction efficiencies seen in other clinical trials. U118 glioblastoma (GBM) and HCC827 lung cancer cell lines were stably transfected with a plasmid expressing Luciferase and tdTomato (−Luc) proteins to allow for tumor cell discrimination. B7-H3.TMI.ζ and B7-H3.28.BB.ζ CAR-T cells showed tumor cell killing against U118-Luc cells (FIG. 4B). B7-H3.28.ζ, B7-H3.TMI.ζ, and B7-H3.28.BB.ζ CAR-T cells showed tumor cell killing against HCC827-Luc cells (FIG. 4C). To confirm our CARs were effective against non-solid tumors as well, we also examined if our CAR-T cells could target the THP-1 acute monocytic leukemia (AML) cell line. B7-H3.TMI.ζ, B7-H3.28.BB.ζ, and B7-H3.TMI.BB.ζ CAR-T cells showed significant tumor cell killing against THP-1 cells (FIG. 4D). Although donor-to-donor variability was high, it was consistently found that B7-H3.TMI.ζ and B7-H3.28.BB.ζ CAR-T cells showed anti-tumor responses across all cell lines tested.

A time-lapse imaging cytotoxicity assay was performed by repeatedly imaging U118-Luc or HCC827-Luc tumor cells alone or cocultured with B7-H3.TMI.ζ, B7-H3.28.BB.ζ, or CD19.28.BB.ζ CAR-T cells to determine if there were differences in the kinetics of tumor cell killing between these two CARs. Live tumor cell growth was tracked based on tdTomato signal and morphological exclusion of dead cells. Against U118-Luc tumor cells, control CD19.28.BB.ζ CAR-T cells showed similar tumor growth to tumor cells alone, whereas both the B7-H3.TMI.ζ and B7-H3.28.BB.ζ CAR-T cells cleared the tumor cells (FIG. 4E). This finding was recapitulated in HCC827-Luc cocultures (FIG. 4F).

The cytokine release profile of B7-H3. TMI.ζ and B7-H3.28.BB.ζ CAR-T cells was evaluated using a multiplexed flow cytometry bead assay measuring the following cytokines: IL-2, IL-4, IL-5, IL-6, IL-9, IL-10, IL-13, IL-17A, IL-17F, IL-22, IFN-γ, and TNF-α. After coculture with HCC827 tumor cells, both B7-H3.TMI.ζ and B7-H3.28.BB.ζ CAR-T cells showed significantly higher cytokine release across all cytokines tested compared to control CD19.28.BB.ζ CAR-T cells (FIG. 4G). B7-H3.TMI.ζ CAR-T cells released significantly less cytokines than B7-H3.28.BB.ζ CAR-T cells across apart from IL-6.

These results demonstrated that B7-H3.TMI.ζ and B7-H3.28.BB.ζ CAR-T cells showed equivalent cytotoxic responses and killing kinetics in vitro. While the cytokine secretion profile of these CAR-T cells was similar, B7-H3.TMI.ζ CAR-T cells mostly secreted lower amounts of these cytokines compared to B7-H3.28.BB.ζ CAR-T cells.

### B7-H3.TMI.ζ CAR-T Cells Show Anti-Tumor Responses In Vivo

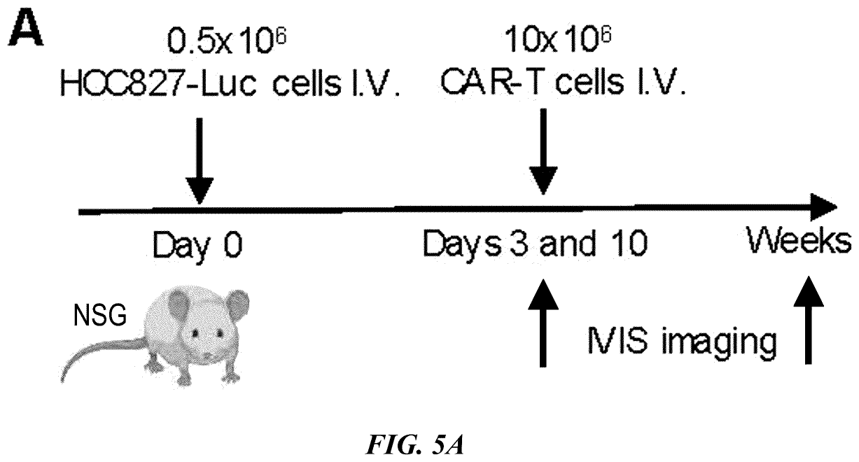

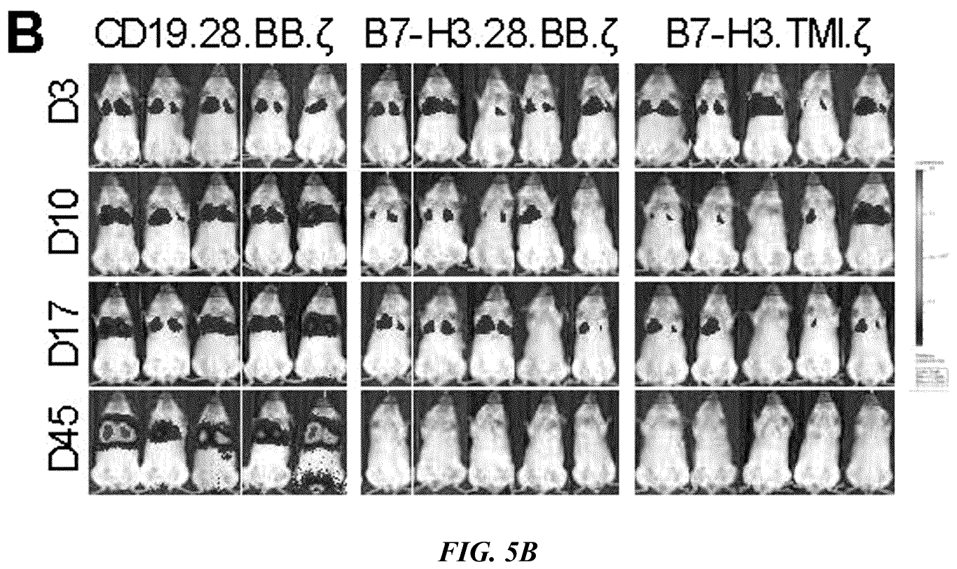

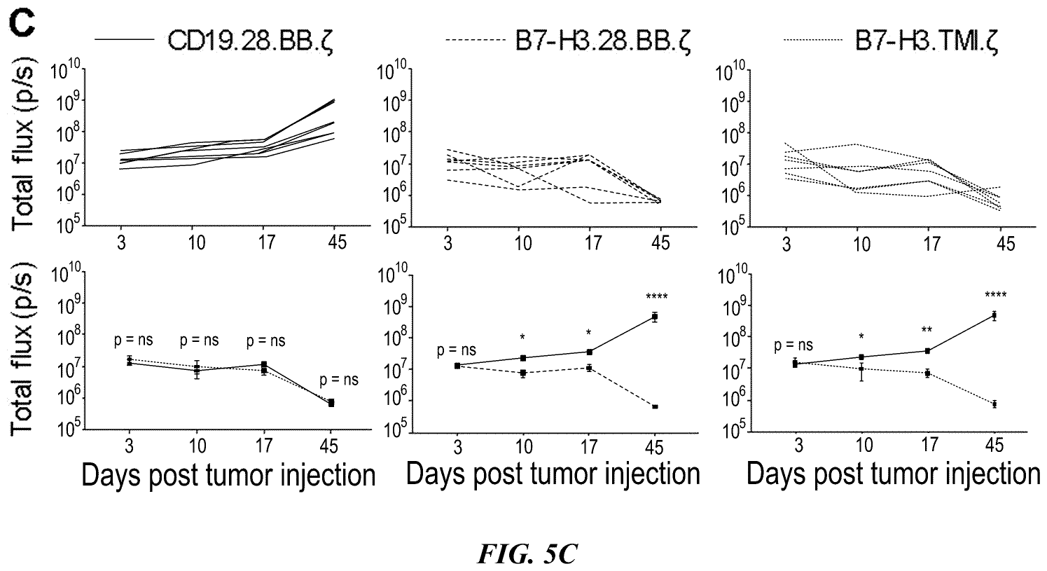

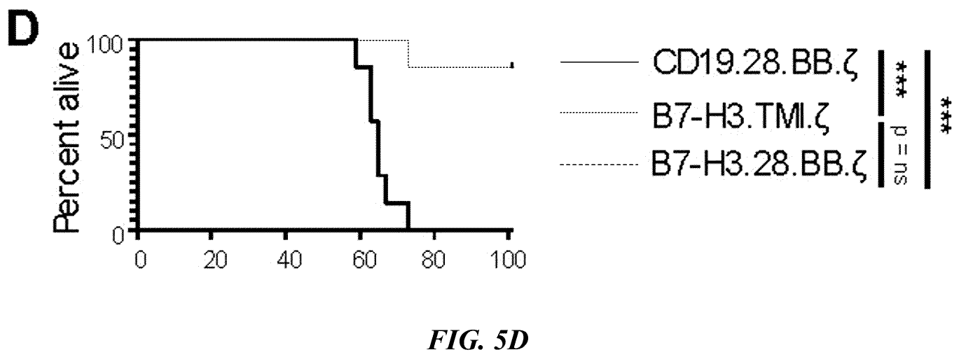

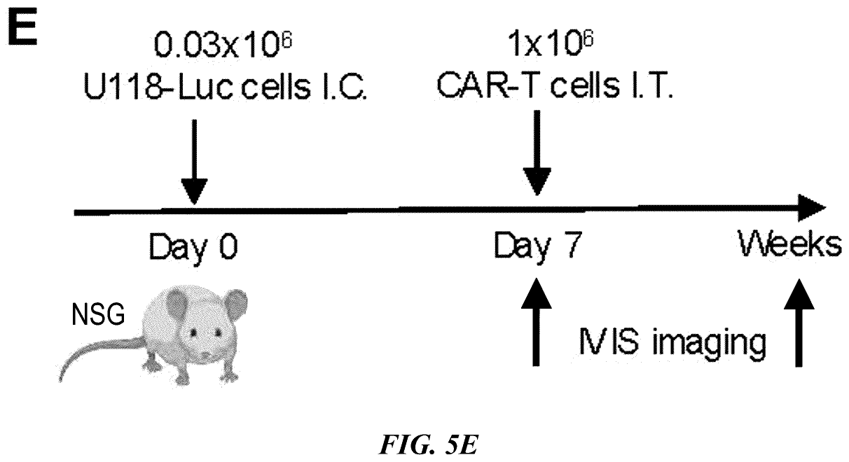

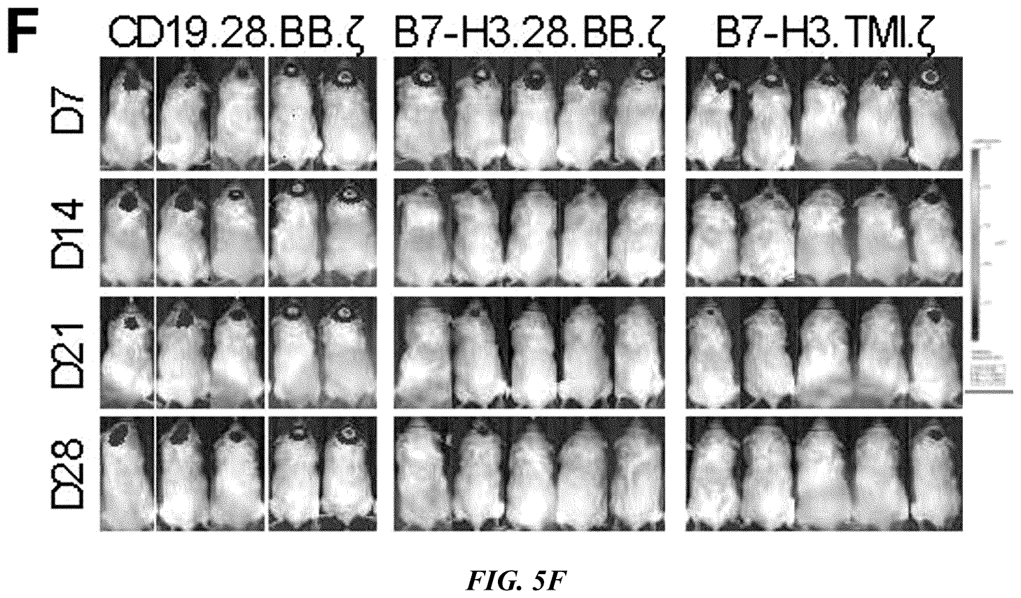

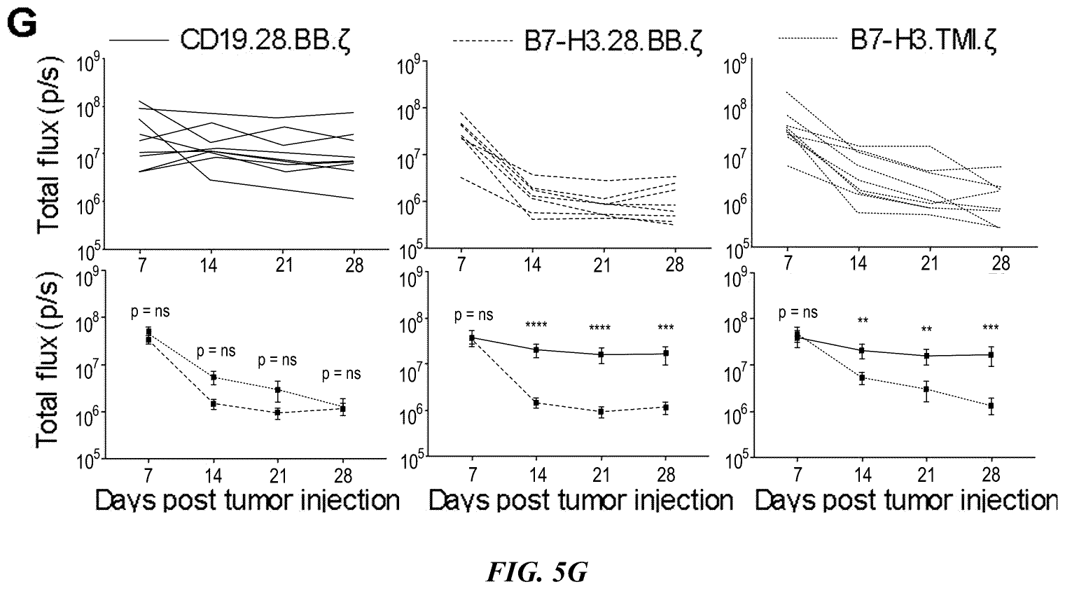

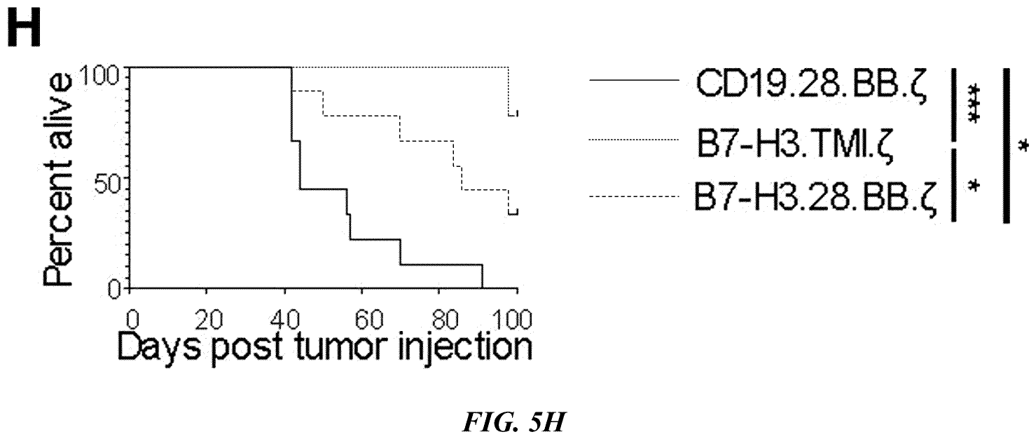

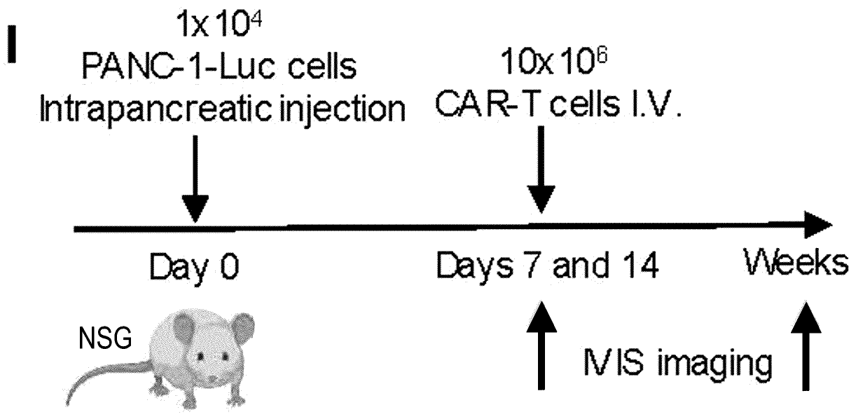

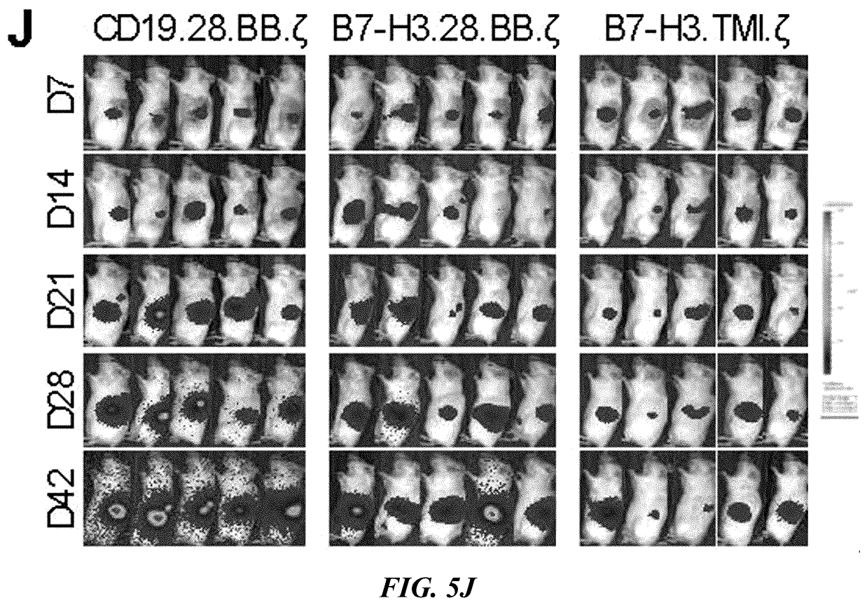

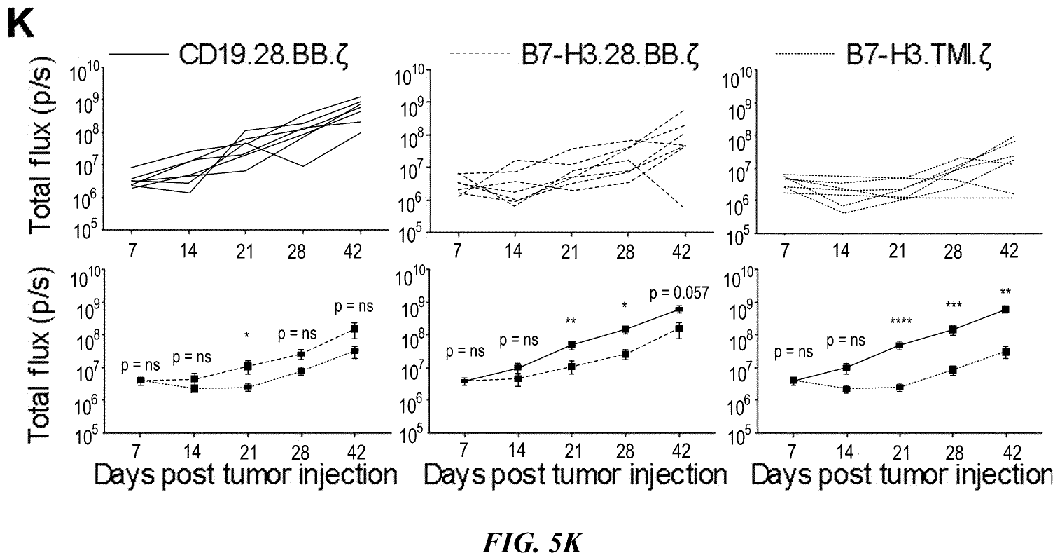

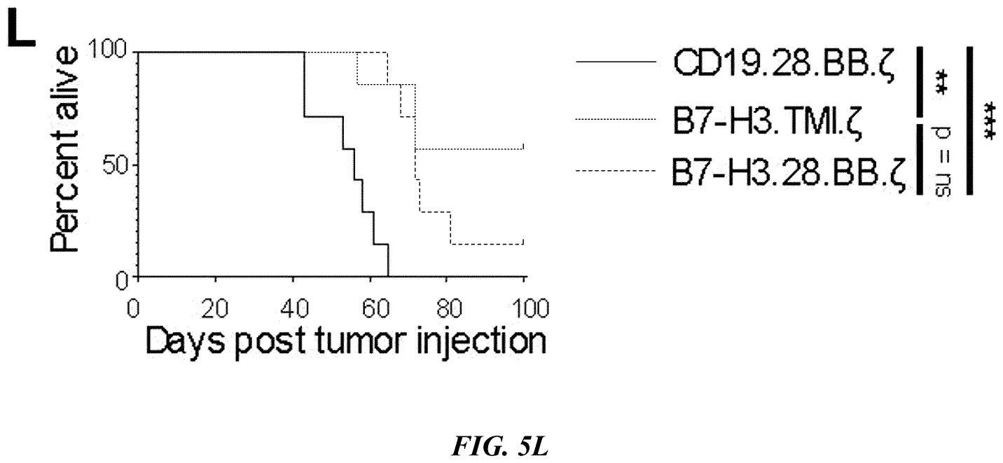

The in vivo anti-tumor responses of the lead B7-H3 CARS, B7-H3.TMI.ζ and B7-H3.28.BB.ζ, alongside control CD19.28.BB.ζ, were assessed in three solid tumor models. All tumor cell lines (HCC827, U118, and PANC-1) were stably transfected with a plasmid containing Luciferase and tdTomato (−Luc) to allow for in vivo tracking by bioluminescent imaging. The CARs B7-H3.TMI.ζ and B7-H3.28.BB.ζ were tested in a metastatic lung cancer model. HCC827-Luc tumor cells were injected intravenously (I.V.) into NSG mice followed by injection of B7-H3.TMI.ζ, B7-H3.28.BB.ζ, or control CD19.28.BB.ζ CAR-T cells I.V. three and ten days later (FIG. 5A). B7-H3.TMI.ζ and B7-H3.28.BB.ζ CAR-T cells reduced tumor burden and showed a concomitant increase of overall survival compared to CD19.28.BB.ζ CAR-T (FIGS. 5B-5D). There was no significant difference in tumor cell signal or survival between the two B7-H3 CAR-T cells (FIG. 5B-5D).

The CAR-T cell therapy was explored in an orthoptic GBM model. U118-Luc cells were intracranially injected into the right cerebral hemisphere of NSG mice followed by an injection of CAR-T cells intratumorally (I.T.) seven days later (FIG. 5E), as it has been shown that intratumorally injected CAR-T cells confer superior anti-tumor responses than I.V. injected CAR-T cells at equivalents low doses. Both B7-H3.TMI.ζ and B7-H3.28.BB.ζ CAR-T cells showed anti-tumor responses against U118-Luc cells compared to CD19.28.BB.ζ CAR-T cells (FIGS. 5F and 5G). There was no difference in tumor burden between the B7-H3.TMI.ζ and B7-H3.28.BB.ζ CAR-T cells (FIGS. 5F and 5G). While both B7-H3 CAR-T cells improved overall survival compared to CD19 CAR-T cells, B7-H3.TMI.ζ CAR-T cells had superior survival outcomes compared to B7-H3.28.BB.ζ CAR-T cells (FIG. 5H). No significant tumor burden prior to death in B7-H3.28.BB.ζ CAR-T cell-treated mice was observed, suggesting that the tumor was not the cause of death in this cohort of mice.

The CAR-T cell therapy was explored in an orthotopic model of pancreatic cancer. NSG mice with were orthotopically injected with PANC-1-Luc tumor cells into the pancreas, followed by I.V. injections of CAR-T cells seven and fourteen days later (FIG. 5I). Both B7-H3 CAR-T cells reduced tumor burden and improved overall survival compared to control CD19.28.BB.ζ CAR-T cells (FIGS. 5J to 5L). B7-H3.TMI.ζ CAR-T cells had less tumor burden compared to B7-H3.28.BB.ζ CAR-T cells, although this only reached statistical significance on day 21 (FIG. 5K). Similarly, there was a trend towards improved survival in B7-H3.TMI.ζ CAR-T cells compared to B7-H3.28.BB.ζ CAR-T cells, with four of seven B7-H3.TMI.ζ CAR-T cell-treated alive 100 days after tumor cell injection compared to one out of seven B7-H3.28.BB.ζ CAR-T cell-treated mice.

Together, these experiments demonstrate that B7-H3.TMI.ζ and B7-H3.28.BB.ζ CAR-T cells show anti-tumor responses against multiple tumor models in vivo. Further, B7-H3.TMI.ζ CAR-T cells demonstrate equivalent or superior outcomes in a tumor-dependent manner.

### B7-H3.TMI.ζ CAR-T Cells Persist In Vivo

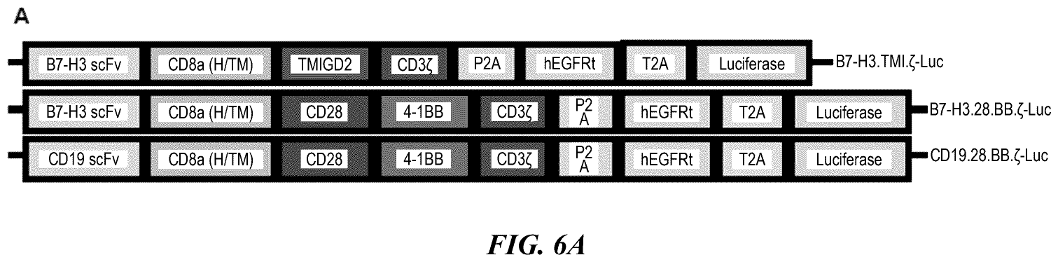

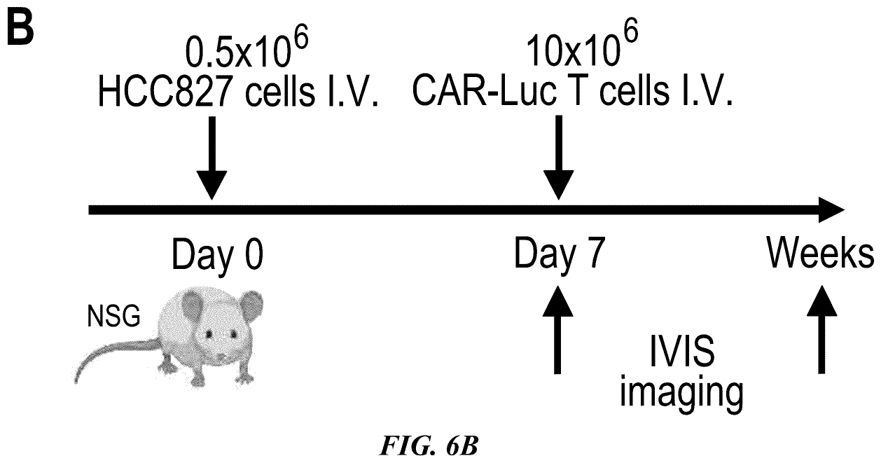

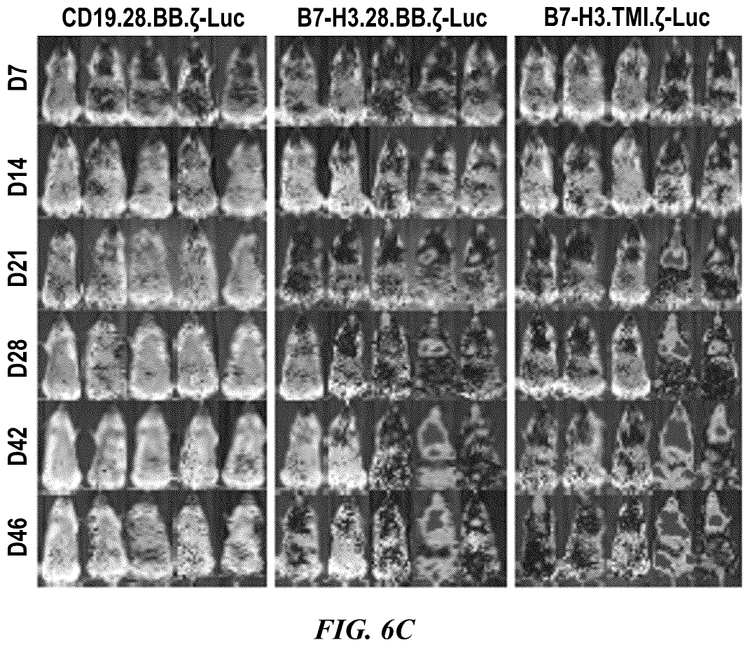

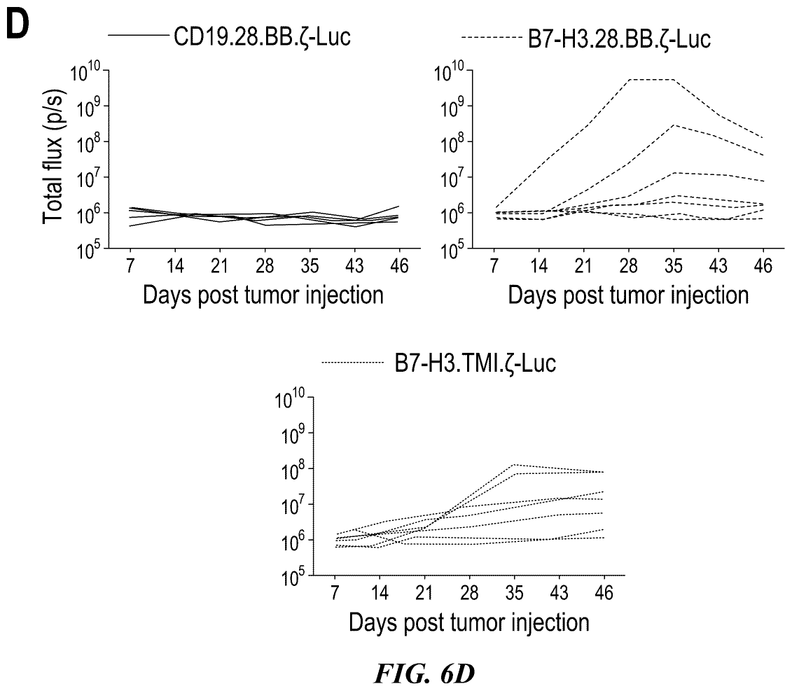

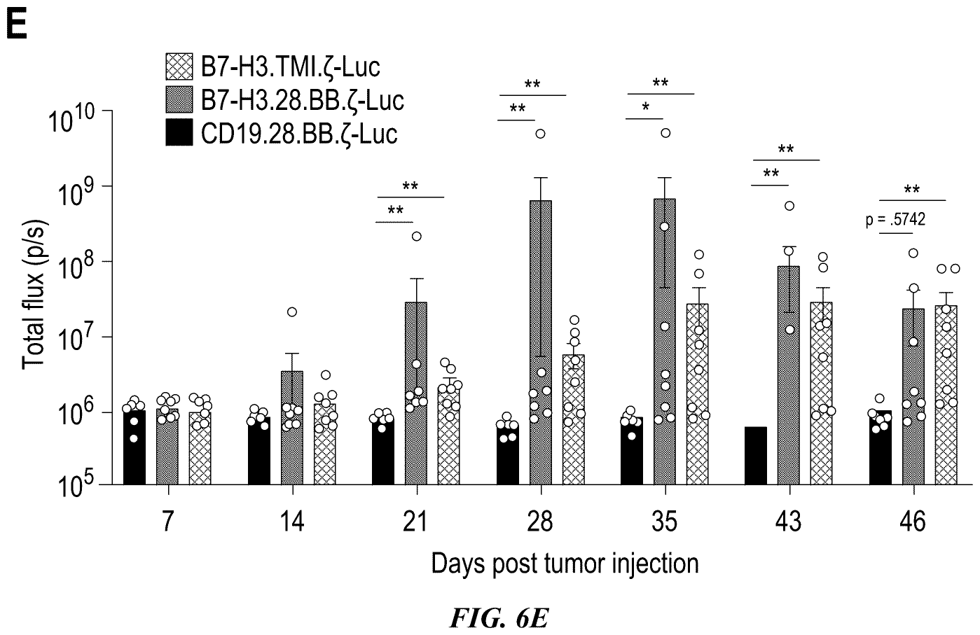

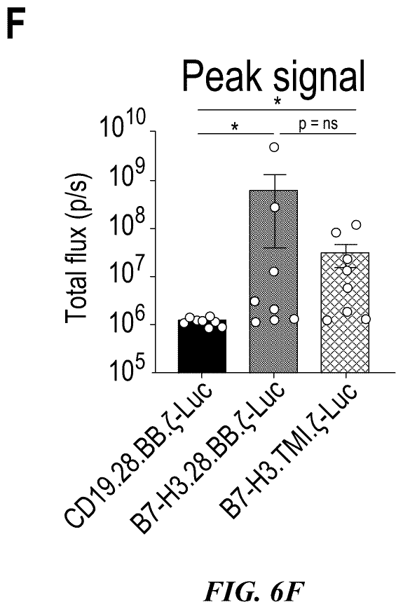

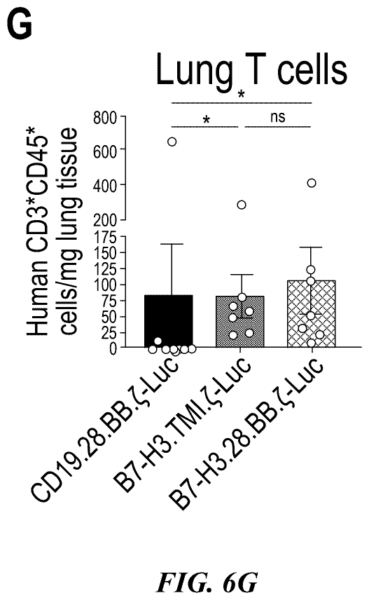

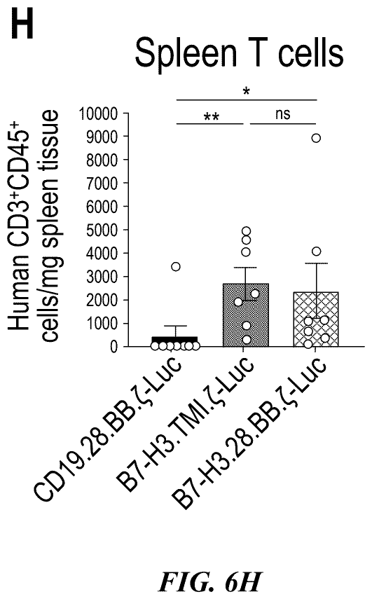

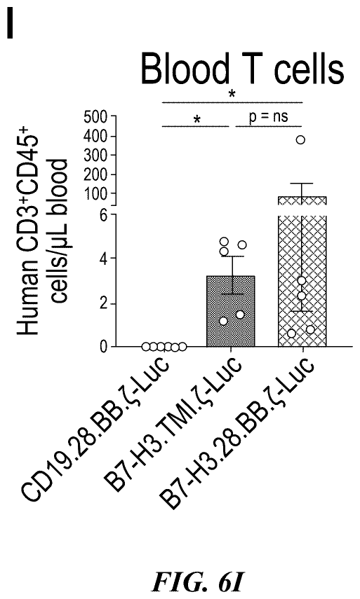

As the B7-H3 CAR-T cells were determined to be cytolytic in multiple tumor models in vivo, the expansion and persistence of B7-H3 CAR-T cells in vivo was examined. The original CAR constructs were modified to include a self-cleaving T2A peptide followed by a luciferase (CAR-Luc) to allow for in vivo CAR-T cell tracking using bioluminescent imaging (FIG. 6A). NSG mice were injected with parental HCC827 cells that do not express a luciferase, followed by a single, sub-therapeutic injection of B7-H3.TMI.ζ-Luc, B7-H3.28.BB.ζ-Luc, or CD19.28.BB.ζ-Luc CAR-T cells I.V. (FIG. 6B). On day 7, CAR-Luc signal was detectable in all constructs and there was no difference between any group; beginning on day 21, both B7-H3 CAR-Luc T cells showed significantly higher signal than CD19 CAR-Luc T cells; however, by day 46 B7-H3.TMI.ζ-Luc but not B7-H3.28.BB.ζ-Luc CAR-T cells showed significantly higher CAR-Luc signal than CD19.28.BB.ζ-Luc CAR-T cells, suggesting that B7-H3.TMI.ζ-Luc CAR-T cells can persist longer that B7-H3.28.BB.ζ-Luc CAR-T cells in vivo, although the latter did approach statistical significance (FIG. 6C to 6E).

Examination of the peak CAR-Luc signal from each mouse throughout the experiment revealed that B7-H3.TMI.ζ-Luc and B7-H3.28.BB.ζ-Luc CAR-T cells showed equivalent expansion that was higher than CD19.28.BB.ζ-Luc CAR-T cells (FIG. 6F). Examining the number of T cells in the lungs, spleen, and blood of CAR-Luc treated mice at the end of the experiment revealed that B7-H3-CAR-Luc T cells were present in greater numbers than CD19.28.BB.ζ-Luc CAR-T cells, and at equivalent numbers between the B7-H3 CAR-Luc T cell constructs (FIG. 6G to 6I). Taken together, these data show that both B7-H3 CAR Luc-T cells can expand and persist in vivo in an antigen-dependent manner. Further, B7-H3.TMI.ζ-Luc CAR-T cells show modestly improved persistence compared to B7-H3.28.BB.ζ-Luc CAR-T at late timepoints, likely due to CAR persistence in locations other than the lungs, spleen, and blood.

### B7-H3.TMI.ζ and B7-H3.28.BB.ζ CAR-T Cells Show Transcriptomic Differences In Vitro

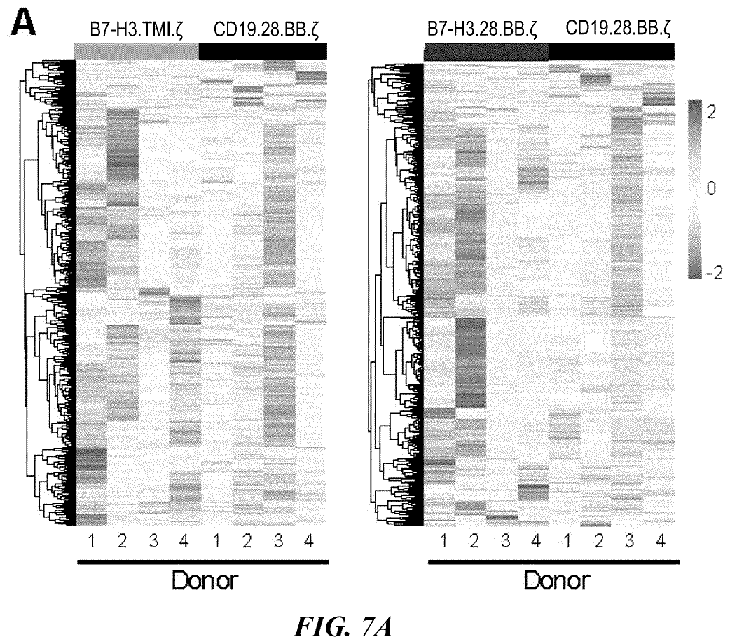

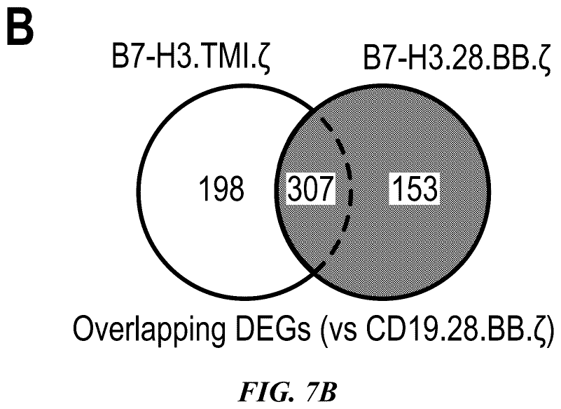

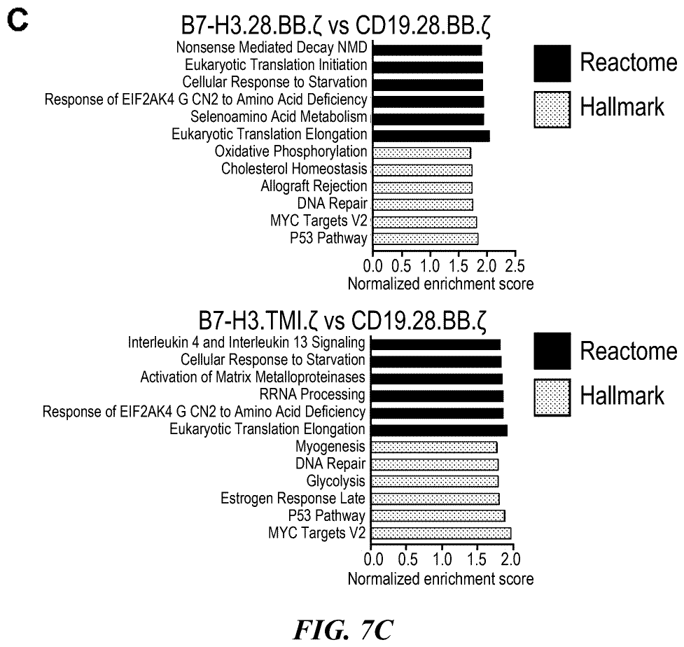

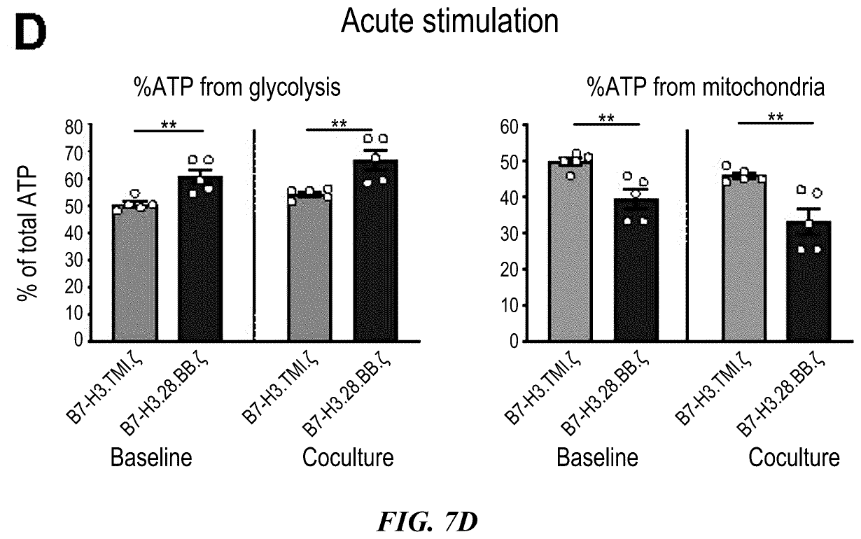

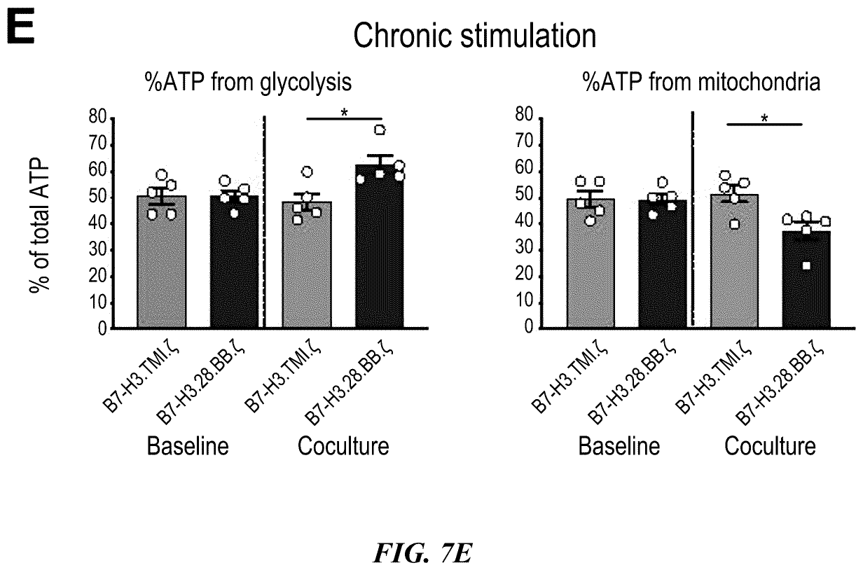

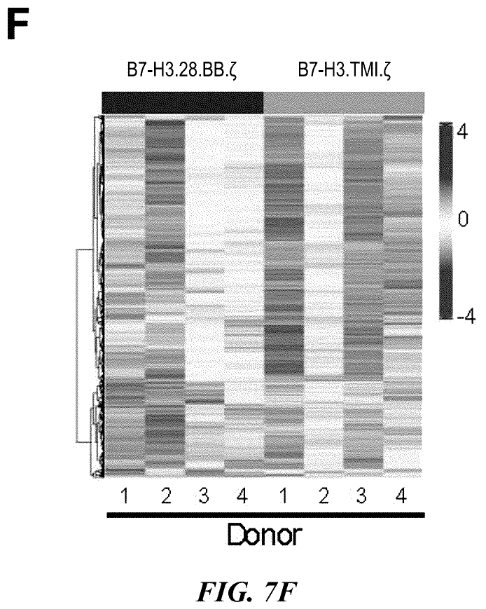

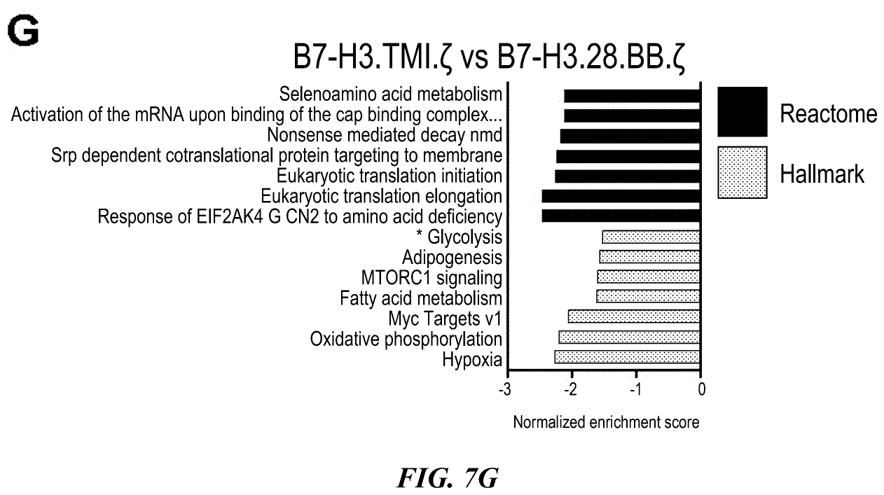

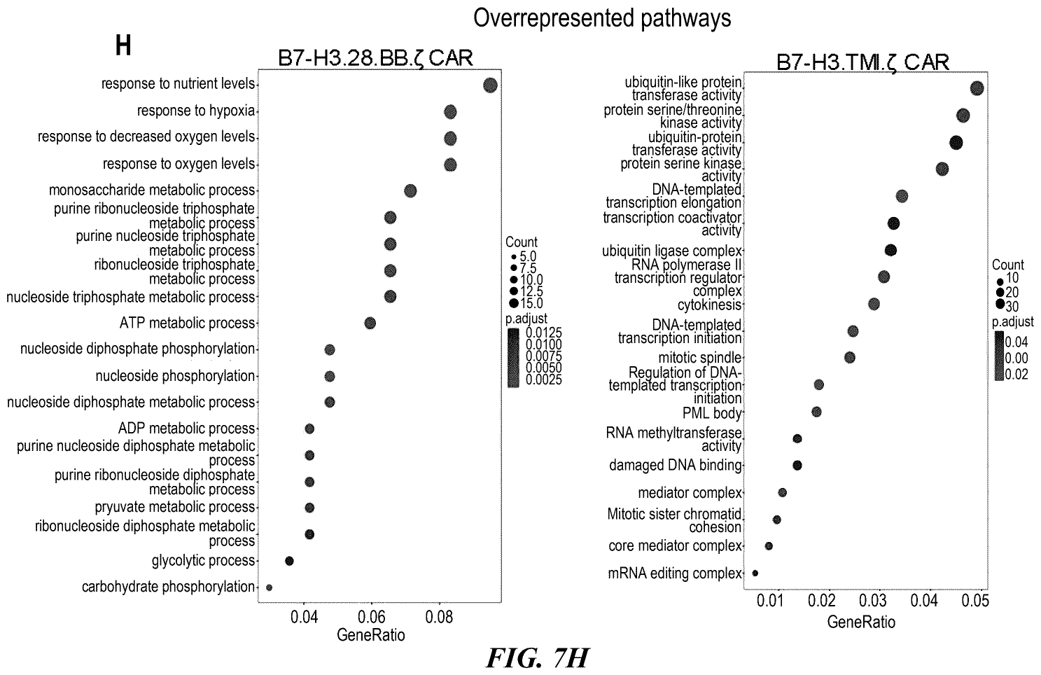

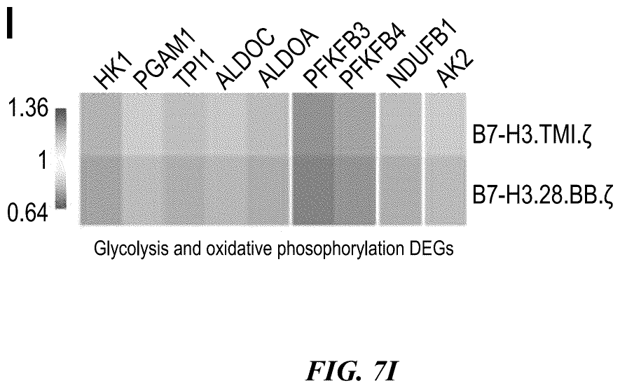

RNA sequencing comparing B7-H3.TMI.ζ and B7-H3.28.BB.ζ CAR-T cells to CD19.28.BB.ζ CAR-T cells after coculture with HCC827 tumor cells for 24 hours in vitro was performed. Compared to CD19.28.BB.ζ CAR-T cells, both B7-H3.TMI.ζ and B7-H3.28.BB.ζ CAR-T cells showed broad transcriptomic differences (FIG. 7A). 307 shared differentially expressed genes (DEGs) were found, as well as 198 and 153 unique DEGs between the B7-H3.TMI.ζ and B7-H3.28.BB.ζ CAR-T cells, respectively (FIG. 7B). Gene set enrichment analysis (GSEA) revealed many enriched pathways in both B7-H3 CAR-T cells compared to CD19 CAR-T cells, with a high degree of similarity in terms of the pathways expressed, but variability in the order of their enrichment (FIG. 7C). Interestingly, among the top enriched pathways, the “Oxidative phosphorylation” pathway in B7-H3.TMI.ζ CAR-T cells was found to be comparable to the “Glycolysis” pathway in the B7-H3.28.BB.ζ CAR-T cells (FIG. 7C), suggesting metabolic differences between these two CAR constructs.

The Seahorse T cell metabolic profiling assay was utilized to measure changes in the % ATP generated from glycolysis and mitochondria after coculture and at baseline (cultured in the absence of tumor cells) to validate the mechanistic findings. In this assay, the % ATP generated from glycolysis and the % ATP generated from mitochondria sum to 100%, thus these values are dependent on one another. Upon acute 24-hour stimulation, B7-H3.TMI.ζ CAR-T cells had a lower % ATP generated from glycolysis (FIG. 7D, left graph) and a concomitant higher % ATP generated from mitochondria (FIG. 7D, right graph) at baseline and after coculture compared to B7-H3.28.BB.ζ CAR-T cells. After chronic stimulation with repeated tumor cell additions, B7-H3.TMI.ζ CAR-T cells did not show differences in the % ATP generated from glycolysis or mitochondria at baseline (FIG. 7E, left graph), but did show a lower % ATP generated from glycolysis and concomitant higher % ATP generated from mitochondria after coculture (FIG. 7E, right graph). It was observed that B7-H3.TMI.ζ CAR-T cells maintained their metabolic signature while B7-H3.28.BB.ζ CAR-T cells modestly increased their glycolytic energy expenditure with an accompanying decrease in mitochondrial energy expenditure (FIGS. 7D and 7E). These functional metabolic assays recapitulated the RNA sequencing experimental findings.

To directly compare B7-H3.TMI.ζ and B7-H3.28.BB.ζ CAR-T cells, an additional RNA sequencing experiment was performed from RNA isolated after 72 hours of coculture with HCC827 tumor cells. Broad transcriptomic differences between these two B7-H3 CARs were present, with a total of 1328 DEGs identified (FIG. 7F). GSEA analysis revealed four metabolic were negatively enriched in B7-H3-TMIGD2 CAR-T cells, including “Oxidative phosphorylation”, “Fatty acid metabolism”, and “Adipogenesis”; “Glycolysis” approached significance (FDR=0.05) (FIG. 7G). These results suggest that B7-H3.28.BB.ζ CAR-T cells were overall more metabolically active than B7-H3.TMI.ζ CAR-T cells after 72 hours of coculture. To determine which pathway(s) contributed the largest role, an overrepresentation analysis (ORA) was performed by examining what pathways were overrepresented among the most highly expressed DEGs (p adj<0.05; fold change >1.5). ORA analysis revealed “Glycolytic process” as the only overrepresented metabolic pathway in B7-H3.28.BB.ζ CAR-T cells, with other pathways broadly describing hypoxia or nucleotide processes. (FIG. 7H). B7-H3.TMI.ζ CAR-T cells broadly showed changes pathways associated with transcription, mitosis, and ubiquitination (FIG. 7H).

Using a previously published gene set describing key enzymes, regulatory proteins, accessory proteins, and other related genes in glycolysis and oxidative phosphorylation, eight upregulated DEGs were found associated with the classical glycolysis enzymes or regulation of glycolysis (HK1, PGAM1, TPI1, ALDOC, ALDOA, PFKFB3, and PFKFB4), compared to only two DEGs associated with a subunit or other function in oxidative phosphorylation (NFDFB1 and AK2) in B7-H3.28.BB.ζ CAR-T cells compared to B7-H3.TMI.ζ CAR-T cells (FIG. 7I). Together, these data suggest that B7-H3.28.BB.ζ CAR-T cells utilize the glycolytic pathway more so than other pathways for their metabolic needs.

### B7-H3.TMI.ζ CAR-T Cells Show Distinct Transcriptional Programs In Vivo

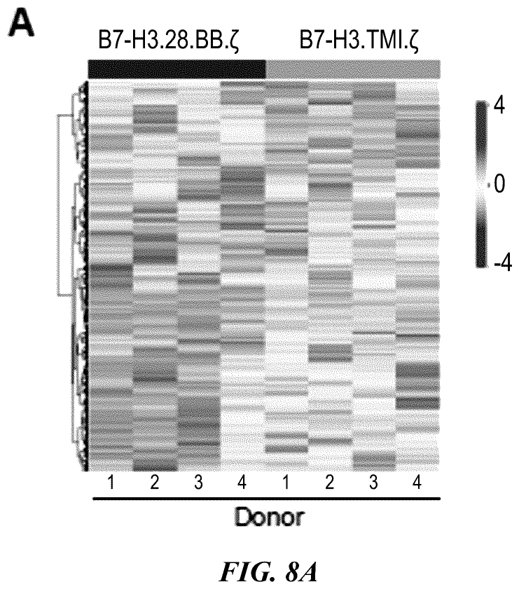

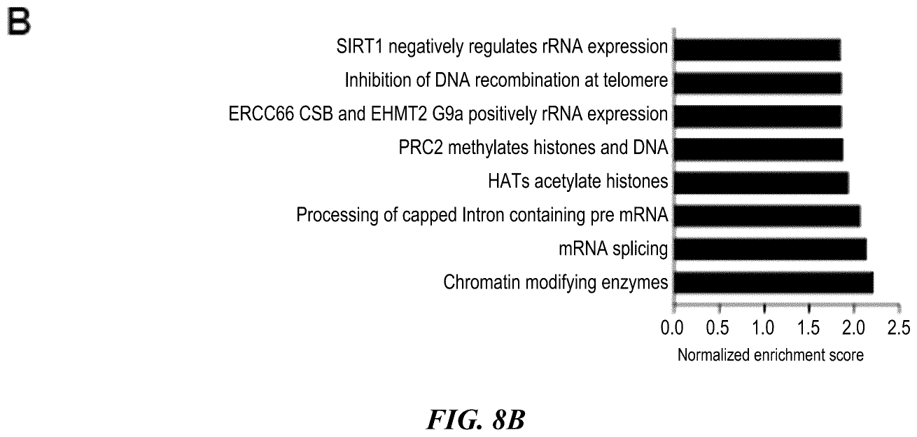

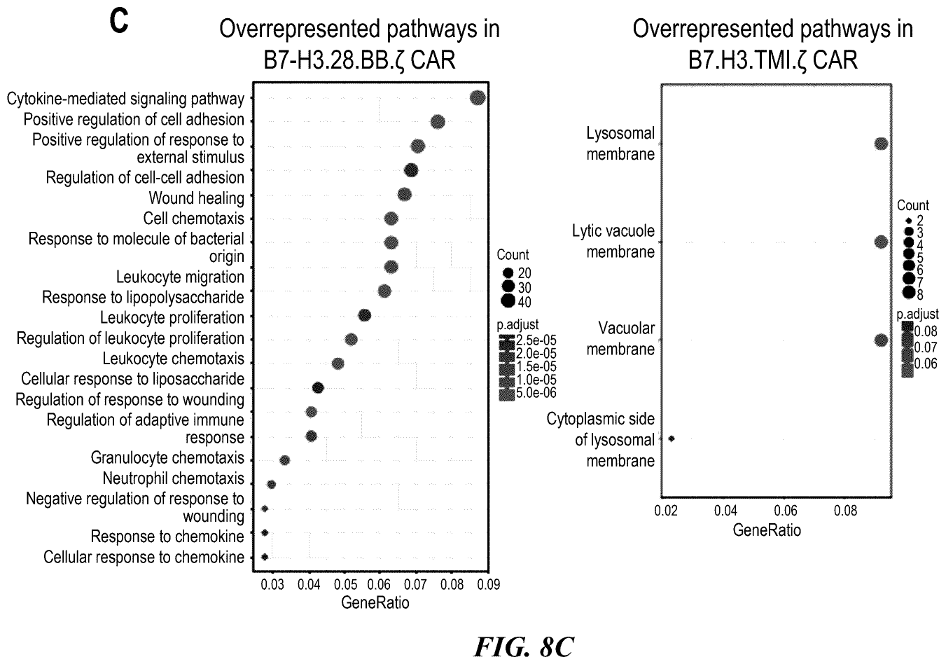

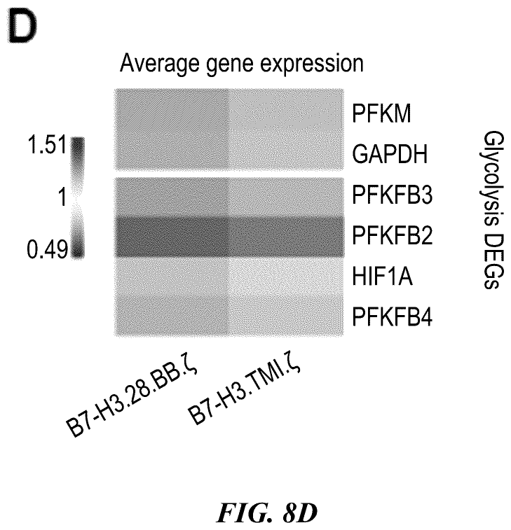

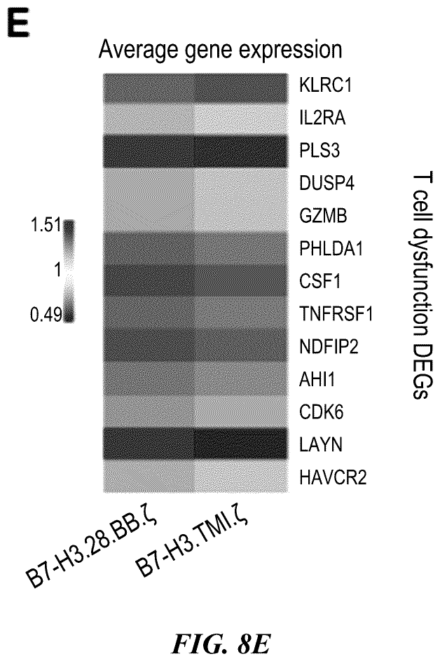

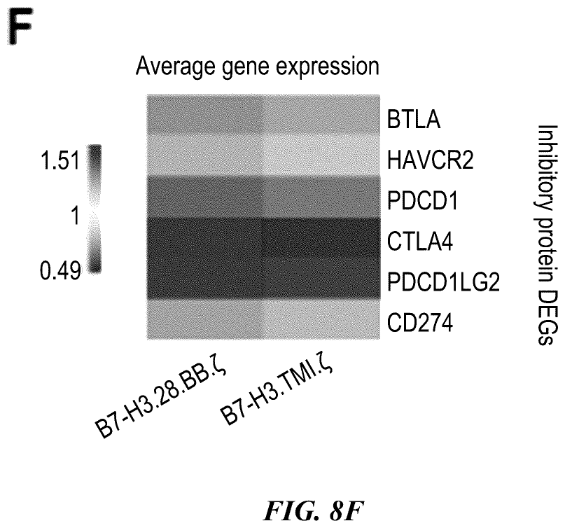

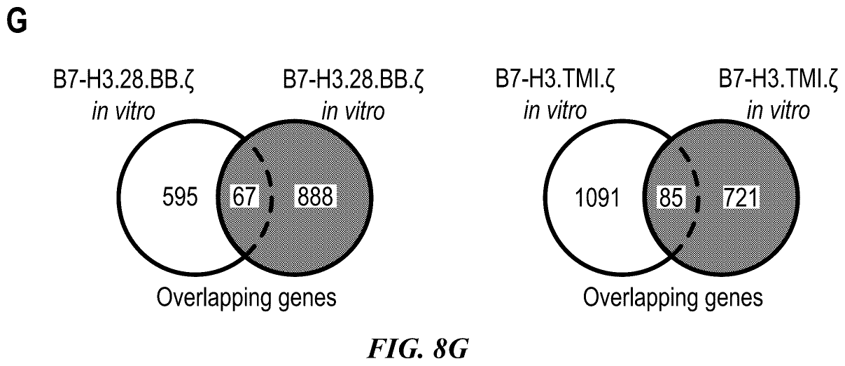

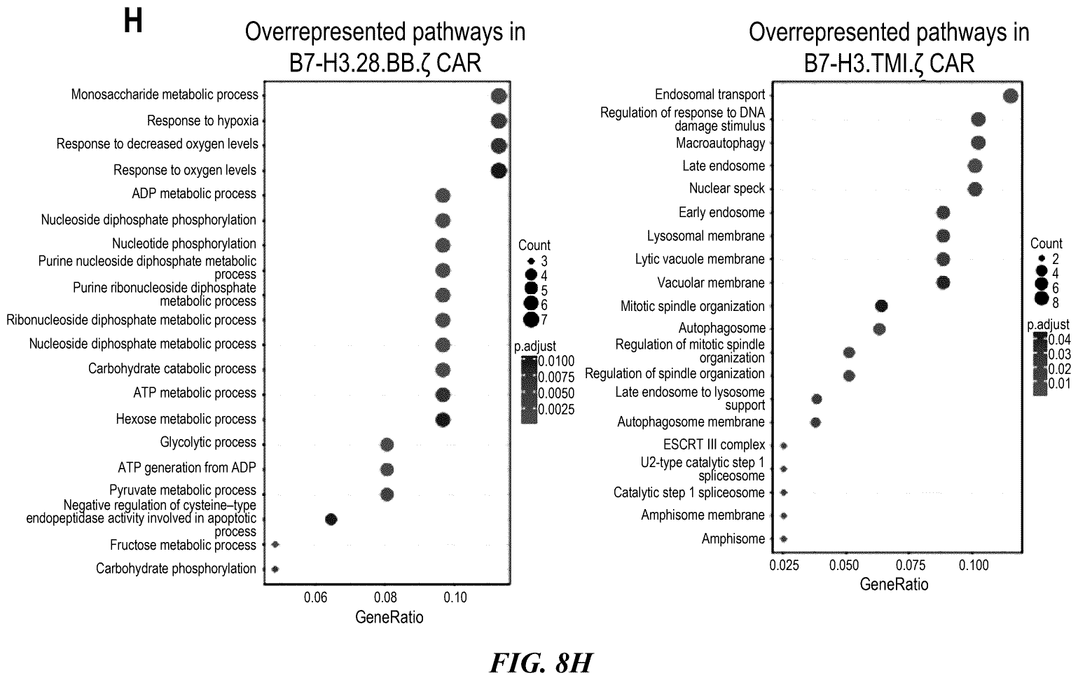

The tumor microenvironment was examined to determine the impact transcriptional programs would have in the B7-H3 CAR-T cells. RNA sequencing was performed from lung-infiltrating T cells collected from the lungs of lung-tumor bearing mice 7 days after B7-H3 CAR-T cell injection. Broad transcriptomic differences were found and 945 DEGs between our B7-H3 CAR-T cells (FIG. 8A). Examining the top enriched pathways by GSEA analysis revealed B7-H3.TMI.ζ CAR T cells were positively enriched for pathways broadly describing RNA- and DNA-associated processes (FIG. 8B). ORA analysis revealed that B7-H3.28.BB.ζ CAR T cells showed overrepresented pathways broadly encompassing cytokines and chemokine pathways among others, and while B7-H3.TMI.ζ CAR T cells did not show statistically significant overrepresented pathways (p adj<0.05) due to few genes meeting the log 2 fold change cutoff, overrepresented pathways approaching significance (p<0.1) described lysosomal and vacuole pathways (FIG. 8C). Using the same metabolism gene set as before, metabolism-associated gene signatures were analyzed in this data set. Six DEGs were found associated with glycolysis enzymes and regulatory proteins upregulated in B7-H3.28.BB.ζ CAR T cells (PFKM, GAPDH, PFKFB3, PFKFB2, HIF1A, and PFKFB4) and no genes associated with oxidative phosphorylation (FIG. 8D).

Using a recently described T cell dysfunction gene signature, 12 DEGs were found upregulated in the B7-H3.28.BB.ζ CAR T cells (IL2RA, PLS3, DUSP4, GZMB, PHLDA1, CSF1, TNFRSF18, NDFIP2, AHI1, CDK6, LAYN, AND HAVCR2) compared to only one in B7-H3.TMI.ζ CAR T cells (KLRC1) (FIG. 8E). Examining a hand-curated gene list of inhibitory proteins, it was found that B7-H3.TMI.ζ CAR T cells showed six downregulated DEGs (BTLA, HAVCR2, PDCD1, CTLA4, PDCDLG2, and CD274), compared to B7-H3.28.BB.ζ CAR T cells (FIG. 8F). Together, these data suggest that B7-H3.TMI.ζ and B7-H3.28.BB.ζ function differently within the in vivo tumor microenvironment, with the former showing a less glycolytic, less dysfunctional, and less inhibited phenotype compared to the latter.

Common significant genes (p adj<0.05) were analyzed between the in vivo and in vitro RNA sequencing experiments that directly compared the two B7-H3 CAR-T cells. 67 shared genes were found in B7-H3.28.BB.ζ CAR and 85 genes in the B7-H3.TMI.ζ CAR (FIG. 8G). ORA analysis of these common genes revealed numerous overrepresented pathways in both CAR constructs (FIG. 8H). In the B7-H3.28.BB.ζ CAR, pathways broadly related to hypoxia and nucleotide metabolism were present among others; notably, “glycolytic process” also appeared in this list. In the B7-H3.TMI.ζ CAR, pathways broadly related to endosomes, lysosomes, autophagy, and others were present. Taken tougher, these results show that, when examining common DEGs across experiments, B7-H3.TMI.ζ and B7-H3.28.BB.ζ CAR-T cells show distinct pathway signatures.

B7-H3.TMI.ζ CAR-T Cells Show Distinct Phenotypic Changes after Chronic Antigen Exposure

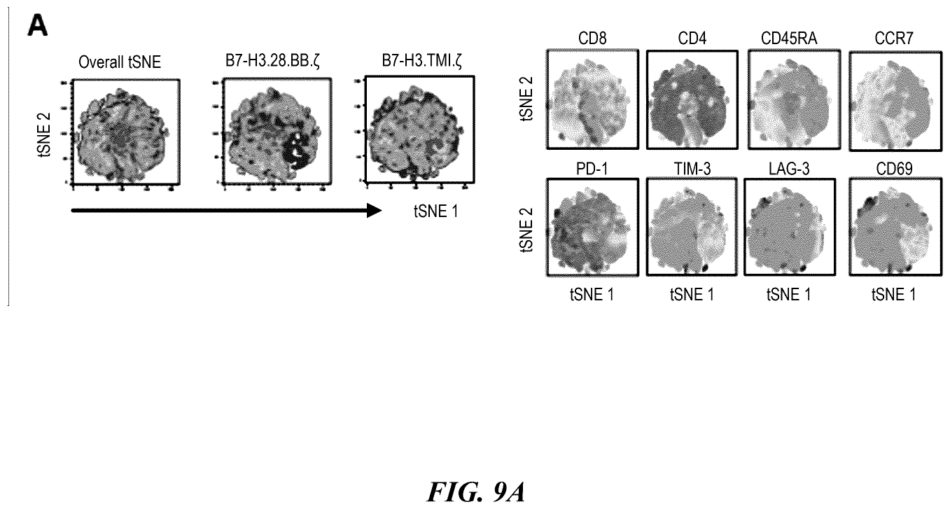

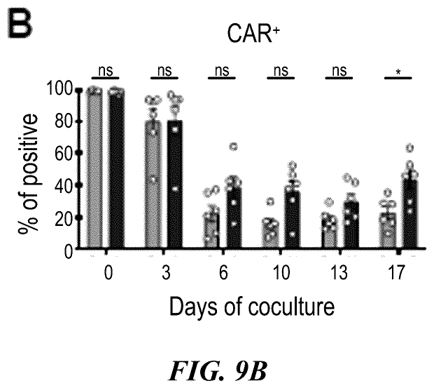

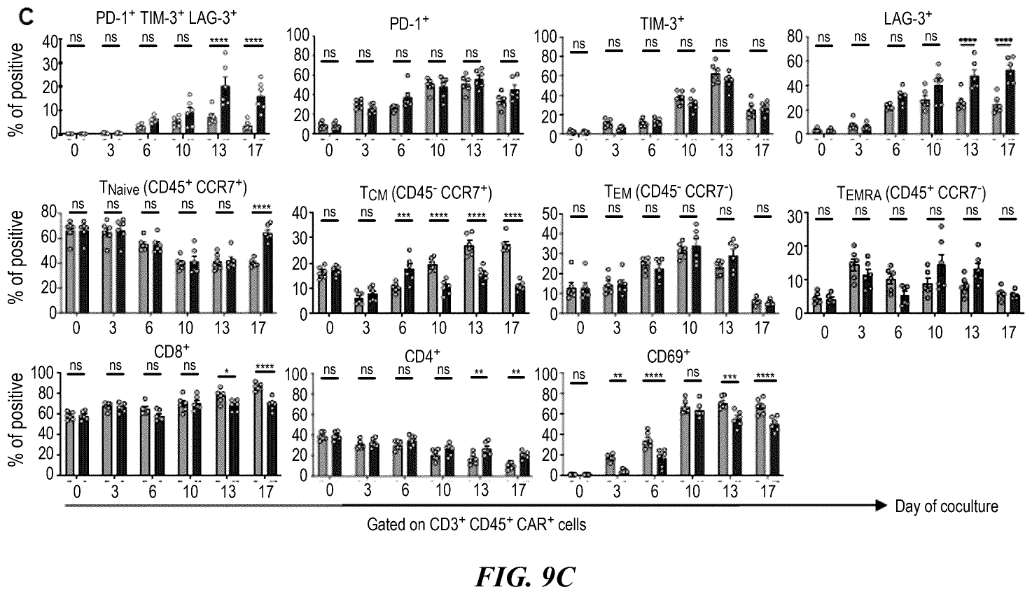

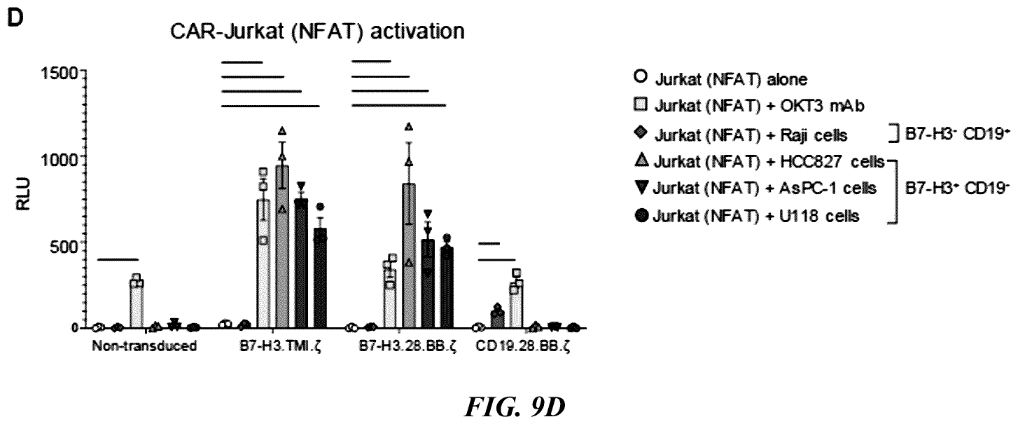

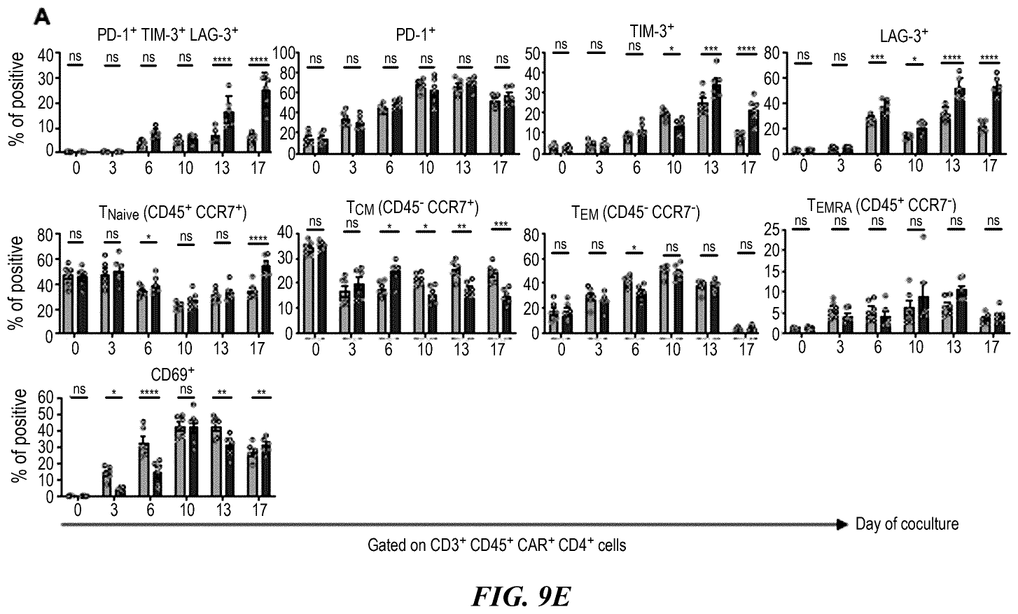

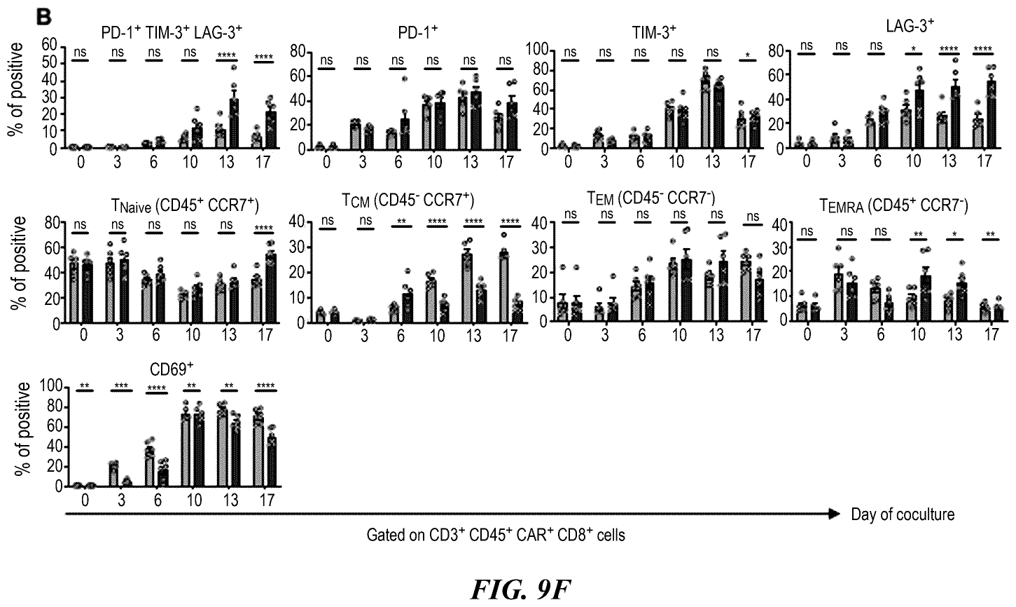

Persistent exposure to antigens can lead to dysfunctional phenotypes and suboptimal effector responses in CAR-T cells. In this setting, different CAR constructions can significantly influence the expression of a variety cell surface markers. An in vitro model of chronic antigen exposure (CAE) was adapted wherein CAR T cells were continuously cultured with sufficient HCC827 tumor cells so that the tumor cells were always present in the coculture to examine if the TMIGD2 and CD28-4-1BB costimulatory domains would also differentially alter cell surface protein expression. The phenotype of CD3+, CD4+, and CD8+ CAR+ B7-H3.TMI.ζ and B7-H3.28.BB.ζ CAR-T cells were compared at each analysis timepoint over the course of 17 days of coculture with HCC827 tumor cells. Describing first the results from the total CD3+ CAR+ population, global changes between the two B7-H3 CAR constructs were observed via t-distributed stochastic neighbor embedding (t-SNE) analysis generated from CAR+ cells at all timepoints (FIG. 9A). CAR expression decreased in both B7-H3 CAR-T cells, with B7-H3.TMI.ζ CAR-T cells showing fewer CAR+ cells on day 17 (FIG. 9B). This finding demonstrates CAR downregulation post-tumor encounter. Looking at PD-1+ TIM-3+ LAG-3+ exhausted CAR-T cells, it was found that B7-H3.TMI.ζ CAR-T cells acquired fewer exhausted cells by days 13 and 17 than B7-H3.28.BB.ζ CAR-T cells (FIG. 9C). As differences in the expression of PD-1, TIM-3, LAG-3, or a combination could underlie this finding, each protein was subsequently individually examined. No differences in PD-1 or TIM-3 expression were detected (FIG. 9C). By contrast, it was found that LAG-3 expression in B7-H3.TMI.ζ CAR T cells was significantly lower on days 13 and 17 compared to B7-H3.28.BB.ζ CAR T cells (FIG. 9C).

When examining memory phenotypes, a significantly enriched population of central memory cells (CD45RA− CCR7+) cells in B7-H3.TMI.ζ CAR-T cells was found beginning on day 10 onwards, while on Day 6 the opposite was true (FIG. 9C). A higher percentage of naïve T cells (CD45R+ CCR7+) in B7-H3.28.BB.ζ were observed on day 17, and no differences in effector memory (CD45RA− CCR7−) or terminally differentiated EMRA cells (CD45RA+ CCR7−) between the groups (FIG. 9C). It was also found that B7-H3.TMI.ζ CAR-T cells exhibited higher CD69+ expression on days 3 and 6, equivalent expression on day 10, and higher expression on days 13 and 17 compared to B7-H3.28.BB.ζ CAR-T cells (FIG. 9C).

It was found that while both B7-H3 CAR-T cells showed a trend of decreasing CD4+ T cells and increasing CD8+ T cells, B7-H3.TMI. (CAR-T cells showed significantly lower CD4+ and significantly higher CD8+ T cells compared to B7-H3.28.BB.ζ CAR-T cells beginning on day 13 (FIG. 9C). The DEGs from the in vivo RNA sequencing data (FIG. 8) were reexamined to validate the finding, given that this experimental design more closely mimics CAE than the other experiments. The CD4 gene was found to be a significant downregulated DEG (p adj<0.05; log 2 fold change >+0.5) and the CD8A gene was significantly upregulated (p adj<0.05; log 2 fold change=0.4206) in B7-H3.TMI.ζ CAR T cells; the CD8B gene showed a non-significant (p adj=0.161; log 2 fold change=0.31) upregulation in B7-H3.TMI.ζ CAR-T cells as well. To confirm this finding at the protein level, lung-infiltrating and splenic T cells were analyzed from lung-tumor bearing mice 46 days post tumor injection. One donor was the same as in FIG. 6, while another was a separate donor. B7-H3.TMI.ζ CAR-T cells showed a higher percentage of CD8+ T cells and lower percent of CD4+ T cells compared to B7-H3.28.BB.ζ CAR-T cells in lung-infiltrating T cells, whereas splenic T cells showed no difference in either population.

The trends described above were largely replicated in CD4+ CAR+ and CD8+ CAR+ populations with only minor changes in temporal dynamics (FIG. 9E-9F) except for notable exceptions described below. B7-H3.TMI.ζ CD4+ CAR+ T cells expressed more TIM-3 day 10, and lower TIM-3 on days 13 and 17 compared to B7-H3.28.BB.ζ CD4+ CAR+ CAR-T cells (FIG. 9E). They also showed equivalent percent of CD69+ cells on day 17 due to a decrease in CD69 expression. B7-H3.TMI.ζ CD8+ CAR+ T cells showed a lower percentage of TEMRA cells on Days 10 and 13 compared to B7-H3.28.BB.ζ CAR-T cells (FIG. 9F).

It has been reported that the antigen affinity and T cell receptor (TCR) signaling strength can alter T cell memory formation in CD8+ T cells, and that TCR signal strength can affect the expression of PD-1 and LAG-3. As the B7-H3 CARs utilize the same scFv and thus share the same antigen affinity, the TMIGD2 and CD28-4-1BB costimulatory domains were examined to determine if they altered T cell activation strength as a mechanism underlying the phenotypic differences described above. A T cell activation reporter cell line, Jurkat (NFAT) cells—which express firefly luciferase via NFAT response elements—was transduced with the B7-H3.TMI.ζ, B7-H3.28.BB.ζ, and CD19.28.BB.ζ CARs. Non-transduced Jurkat (NFAT) cells or CAR-transduced Jurkat (NFAT) cells were cultured either alone, with plate-bound activating anti-CD3 OKT3 antibody, with CD19+ B7-H3-cells (Raji), or with CD19-B7-H3+ cell lines (HCC827, AsPC-1, and U118) (FIG. 9D). All Jurkat (NFAT) cells signaled in response to OKT3 stimulation and did not signal when cultured alone. B7-H3.TMI.ζ- and B7-H3.28.BB.ζ-transduced Jurkat (NFAT) cells signaled in response to HCC827, AsPC-1, and U118 tumor cell lines to broadly similar degrees, but not to Raji cells. Notably, the level was nearly identical for the HCC827 cell line used for CAE stimulation and all RNA sequencing experiments. CD19.28.BB.ζ-transduced Jurkat (NFAT) cells signaled in response to Raji cells but not HCC827, AsPC-1, and U118 tumor cell lines. Taken together, these results show that B7-H3.TMI.ζ CAR-T cells acquire differences in memory, exhaustion, activation, and CD4/CD8 phenotype upon CAE, and that this effect is not mediated by differences in signal strength.

### DISCUSSION

B7-H3.TMI.ζ and B7-H3.28.BB.ζ CAR-T cells were the top performing CARs using in vitro killing assays. B7-H3.TMI.ζ CAR-T cells were also efficacious in multiple solid tumor models, showing equivalent or superior outcomes to B7-H3.28.BB.ζ CAR-T cells. Additionally, B7-H3.TMI.ζ CAR-T cells showed unique transcriptomic, metabolomic, and phenotypic profiles, indicating that TMIGD2 costimulation offers distinct benefits from CD28-41BB costimulation.

Using in vitro killing assays with lowered transduction efficiency, it was found that only B7-H3.TMI.ζ and B7-H3.28.BB.ζ CAR-T cells could kill three different tumor cell lines. TMIGD2 costimulation may be superior to current FDA-approved CD28 and 4-1BB costimulatory domains. Comparing the two lead constructs, the B7-H3.TMI.ζ CAR-T cells released a lower concentration of cytokines. Given that cytokine release syndrome (CRS) may be mediated in part by cytokines released from CAR-T cells or cells activated by CAR-T cells (e.g. macrophages and monocytes), TMIGD2-based CARs may represent a safer costimulatory domain as well.

Unexpectedly, combining TMIGD2 and 4-1BB signaling using a third-generation CAR did not show cytotoxicity against two solid tumor cells lines (HCC827 and U118). Jurkat (NFAT) cells transduced with our B7-H3.TMI.BB.ζ CAR showed reduced activation compared to B7-H3.28.BB.ζ CAR-transduced cells.

In the orthotopic GBM model, but not other tumor models, it was noted an unexpected toxicity in mice treated with B7-H3.28.BB.ζ CAR-T cells but not B7-H3.TMI.ζ CAR-T cells, suggesting that the latter is safer in this context. As these mice died without any significant tumor burden, it is likely that the CAR-T cells rather than the tumor cells were the underlying cause. As B7-H3.28.BB.ζ CAR-T cells exhibit significantly higher levels of cytokine release than B7-H3.TMI.ζ CAR-T cells, localized cytokine release syndrome may be a factor. A similar effect has been reported in the clinic, described as a “local” or “compartmental” cytokine release syndrome. This toxicity could also be magnified by the I.T. injection of CAR-T cells in this model compared to I.V. administration.

CAR costimulatory domains have significant impacts on CAR-T cell metabolism. CAR-T cells with a CD28 costimulatory domain utilize glycolytic metabolism, whereas a 4-1BB costimulatory domain utilizes oxidative metabolism. Further, analysis of a CD19 CAR-T cell product from one clinical trial has demonstrated that non-responders and partial responders show enrichment of glycolysis gene signatures. In the constructs, B7-H3.28.BB.ζ CAR-T cells showed a metabolic profile more reliant on glycolysis than B7-H3.TMI.ζ CAR-T cells based on RNA sequencing pathway analysis, overrepresentation analysis, acute and chronic stimulation Seahorse metabolic assays, and analysis of common DEGs between RNA sequencing experiments.

Compared to B7-H3.28.BB.ζ CAR-T cells, B7-H3.TMI.ζ CAR-T cells reduced dysfunction- and exhaustion-associated phenotypes which are involved in CAR-T cell anti-tumor responses, and can be modified by the choice of costimulatory domain. Therefore, TMIGD2 costimulation is a new method to prevent T cell dysfunction and exhaustion.

It was found that upon CAE, but not during the initial generation, B7-H3. TMI.ζ CAR-T cells showed a time-dependent enrichment of central memory cells. A higher percentage of this population is associated with better outcomes in CAR-T cell therapy. In addition, CAR-T cells generated from bulk CD8+ T cells compared to central memory-enriched populations show an increased risk for CRS. The TMIGD2 costimulatory domain can increase central memory cells and may be beneficial for improving therapeutic efficacy and safety.

An unexpected enrichment of CD8+ T cells in B7-H3.TMI.ζ CAR-T cells was found compared to the B7-H3.28.BB.ζ CAR-T cells upon CAE. This finding was consistent with phenotypic analysis of in vivo lung-infiltrating T cells and re-analysis of in vivo RNA sequencing gene expression data. CD8+ CAR-T cells make up a larger population than CD4+ CAR-T cells after infusion of bulk CAR-T cell products and after infusion of CAR-T cells products with a 1:1 ratio of CD8:CD4 T cells. TMIGD2 costimulation improves the proliferation of CD8+ CAR-T cells compared to CD28-4-1BB costimulation and/or TMIGD2 costimulation improves the survival of CD8+ CAR-T cells.

### REFERENCES

1. Crespo, J., et al. Phenotype and tissue distribution of CD28H(+) immune cell subsets. Oncoimmunology 6, e1362529 (2017).
2. Janakiram, M., et al. Expression, clinical significance, and receptor identification of the newest B7 family member HHLA2 protein. Clin Cancer Res 21, 2359-2366 (2015).
3. Janakiram, M., Chinai, J. M., Zhao, A., Sparano, J. A. & Zang, X. HHLA2 and TMIGD2: new immunotherapeutic targets of the B7 and CD28 families. Oncoimmunology 4, e1026534 (2015).
4. Janakiram, M., et al. The third group of the B7-CD28 immune checkpoint family: HHLA2, TMIGD2, B7x, and B7-H3. Immunol Rev 276, 26-39 (2017).
5. Zhao, R., et al. HHLA2 is a member of the B7 family and inhibits human CD4 and CD8 T-cell function. Proc Natl Acad Sci USA 110, 9879-9884 (2013).
6. Zhu, Y., et al. B7-H5 costimulates human T cells via CD28H. Nat Commun 4, 2043 (2013).
7. Subramanian, A. et al. Gene set enrichment analysis: A knowledge-based approach for interpreting genome-wide expression profiles. PNAS 102 (43), 15545-15550 (2005). [Hallmark]
8. Liberzon, A. et al. Molecular signatures database (MSigDB) 3.0 Bioinformatics 27 (12), 1739-1740 (2011). [Hallmark]
9. Liberzon, A. et al. The molecular signatures database (MSigDB) hallmark gene set collection Cell Syst. 1 (6), 417-425 (2015). [Hallmark]
10. Gillespie, M. et al. The reactome pathway knowledgebase 2022 Nucleic Acid Research 50 (D1), D687-D692 (2022). [Reactome]
11. Griss, J. et al. ReactomeGSA—Efficient multi-omics comparative pathway analysis Mol. Cell Proteomics 19 (12), 2115-2125 (2020). [Reactome]
12. Jassal, B. et al. The reactome pathway knowledgebase Nucleic Acids Res. 48 (D1), D498-D503 (2020). [Reactome]
13. Wu, G. and Haw, R. Functional interaction network construction and analysis for disease discovery Methods Mol. Biol. 1558, 235-253 (2017). [Reactome]
14. Neelapu S S, Locke F L, Bartlett N L, Lekakis L J, Miklos D B, Jacobson C A, et al. Axicabtagene Ciloleucel CAR T-Cell Therapy in Refractory Large B-Cell Lymphoma. New England Journal of Medicine. 2017; 377 (26): 2531-44.
15. Laetsch T W, Maude S L, Rives S, Hiramatsu H, Bittencourt H, Bader P, et al. Three-Year Update of Tisagenlecleucel in Pediatric and Young Adult Patients With Relapsed/Refractory Acute Lymphoblastic Leukemia in the ELIANA Trial. J Clin Oncol. 2023; 41 (9): 1664-9.
16. Rodriguez-Otero P, Ailawadhi S, Arnulf B, Patel K, Cavo M, Nooka A K, et al. Ide-cel or Standard Regimens in Relapsed and Refractory Multiple Myeloma. New England Journal of Medicine. 2023; 388 (11): 1002-14.
17. Chen Y-J, Abila B, and Mostafa Kamel Y. CAR-T: What Is Next? Cancers. 2023; 15 (3): 663.
18. O'Rourke D M, Nasrallah M P, Desai A, Melenhorst J J, Mansfield K, Morrissette J J D, et al. A single dose of peripherally infused EGFRvIII-directed CAR T cells mediates antigen loss and induces adaptive resistance in patients with recurrent glioblastoma. Sci Transl Med. 2017; 9(399).
19. Goff S L, Morgan R A, Yang J C, Sherry R M, Robbins P F, Restifo N P, et al. Pilot Trial of Adoptive Transfer of Chimeric Antigen Receptor-transduced T Cells Targeting EGFRvIII in Patients With Glioblastoma. J Immunother. 2019; 42 (4): 126-35.
20. Kershaw M H, Westwood J A, Parker L L, Wang G, Eshhar Z, Mavroukakis S A, et al. A phase I study on adoptive immunotherapy using gene-modified T cells for ovarian cancer. Clin Cancer Res. 2006; 12 (20 Pt 1): 6106-15.
21. Zhang C, Wang Z, Yang Z, Wang M, Li S, Li Y, et al. Phase I Escalating-Dose Trial of CAR-T Therapy Targeting CEA (+) Metastatic Colorectal Cancers. Mol Ther. 2017; 25 (5): 1248-58.
22. Hou A J, Chen L C, and Chen Y Y. Navigating CAR-T cells through the solid-tumour microenvironment. Nature Reviews Drug Discovery. 2021; 20 (7): 531-50.
23. Weinkove R, George P, Dasyam N, and Mclellan A D. Selecting costimulatory domains for chimeric antigen receptors: functional and clinical considerations. Clin Transl Immunology. 2019; 8 (5): e1049.
24. Long A H, Haso W M, Shern J F, Wanhainen K M, Murgai M, Ingaramo M, et al. 4-1BB costimulation ameliorates T cell exhaustion induced by tonic signaling of chimeric antigen receptors. Nat Med. 2015; 21 (6): 581-90.
25. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet. 2015; 385 (9967): 517-28.
26. Ying Z, He T, Wang X, Zheng W, Lin N, Tu M, et al. Parallel Comparison of 4-1BB or CD28 Co-stimulated CD19-Targeted CAR-T Cells for B Cell Non-Hodgkin's Lymphoma. Mol Ther Oncolytics. 2019; 15:60-8.
27. Nehama D, Di lanni N, Musio S, Du H, Patane M, Pollo B, et al. B7-H3-redirected chimeric antigen receptor T cells target glioblastoma and neurospheres. EBioMedicine. 2019; 47:33-43.
28. Kawalekar O U, O'Connor R S, Fraietta J A, Guo L, McGettigan S E, Posey A D, Jr., et al. Distinct Signaling of Coreceptors Regulates Specific Metabolism Pathways and Impacts Memory Development in CAR T Cells. Immunity. 2016; 44 (2): 380-90.
29. Drent E, Poels R, Ruiter R, van de Donk N, Zweegman S, Yuan H, et al. Combined CD28 and 4-1BB Costimulation Potentiates Affinity-tuned Chimeric Antigen Receptor-engineered T Cells. Clin Cancer Res. 2019; 25 (13): 4014-25.
30. Zhao X, Yang J, Zhang X, Lu X A, Xiong M, Zhang J, et al. Efficacy and Safety of CD28− or 4-1BB-Based CD19 CAR-T Cells in B Cell Acute Lymphoblastic Leukemia. Mol Ther Oncolytics. 2020; 18:272-81.
31. Tian Y, Sun Y, Gao F, Koenig M R, Sunderland A, Fujiwara Y, et al. CD28H expression identifies resident memory CD8+ T cells with less cytotoxicity in human peripheral tissues and cancers. Oncoimmunology. 2019; 8 (2): e1538440.
32. Xiao X, Huang S, Chen S, Wang Y, Sun Q, Xu X, et al. Mechanisms of cytokine release syndrome and neurotoxicity of CAR T-cell therapy and associated prevention and management strategies. Journal of Experimental & Clinical Cancer Research. 2021; 40 (1): 367.
33. Picarda E, Ohaegbulam K C, and Zang X. Molecular Pathways: Targeting B7-H3 (CD276) for Human Cancer Immunotherapy. Clin Cancer Res. 2016; 22 (14): 3425-31.
34. Wang X, Chang W C, Wong C W, Colcher D, Sherman M, Ostberg J R, et al. A transgene-encoded cell surface polypeptide for selection, in vivo tracking, and ablation of engineered cells. Blood. 2011; 118 (5): 1255-63.
35. Spiegel J Y, Patel S, Muffly L, Hossain N M, Oak J, Baird J H, et al. CAR T cells with dual targeting of CD19 and CD22 in adult patients with recurrent or refractory B cell malignancies: a phase 1 trial. Nature Medicine. 2021; 27 (8): 1419-31.
36. Zhang Y, Li C, Du M, Jiang H, Luo W, Tang L, et al. Allogenic and autologous anti-CD7 CAR-T cell therapies in relapsed or refractory T-cell malignancies. Blood Cancer J. 2023; 13 (1): 61.
37. Theruvath J, Sotillo E, Mount C W, Graef C M, Delaidelli A, Heitzeneder S, et al. Locoregionally administered B7-H3-targeted CAR T cells for treatment of atypical teratoid/rhabdoid tumors. Nat Med. 2020; 26 (5): 712-9.
38. Chen W, Yang Z, and Chen Y. A Novel Oxidative Phosphorylation-Associated Gene Signature for Prognosis Prediction in Patients with Hepatocellular Carcinoma. Dis Markers. 2022; 2022:3594901.
39. Kankeu Fonkoua L A, Sirpilla O, Sakemura R, Siegler E L, and Kenderian S S. CAR T cell therapy and the tumor microenvironment: Current challenges and opportunities. Molecular Therapy-Oncolytics. 2022; 25:69-77.
40. Good C R, Aznar M A, Kuramitsu S, Samareh P, Agarwal S, Donahue G, et al. An NK-like CAR T cell transition in CAR T cell dysfunction. Cell. 2021; 184 (25): 6081-100.e26.
41. Schoutrop E, Poiret T, El-Serafi I, Zhao Y, He R, Moter A, et al. Tuned activation of MSLN-CAR T cells induces superior antitumor responses in ovarian cancer models. Journal for Immuno Therapy of Cancer. 2023; 11 (2): e005691.
42. Selli M E, Landmann J H, Terekhova M, Lattin J, Heard A, Hsu Y S, et al. Costimulatory domains direct distinct fates of CAR-driven T-cell dysfunction. Blood. 2023; 141 (26): 3153-65.
43. Caruso H G, Hurton L V, Najjar A, Rushworth D, Ang S, Olivares S, et al. Tuning Sensitivity of CAR to EGFR Density Limits Recognition of Normal Tissue While Maintaining Potent Antitumor Activity. Cancer Res. 2015; 75 (17): 3505-18.
44. Li W, Qiu S, Chen J, Jiang S, Chen W, Jiang J, et al. Chimeric Antigen Receptor Designed to Prevent Ubiquitination and Downregulation Showed Durable Antitumor Efficacy. Immunity. 2020; 53 (2): 456-70.e6.
45. Solouki S, Huang W, Elmore J, Limper C, Huang F, and August A. TCR Signal Strength and Antigen Affinity Regulate CD8(+) Memory T Cells. J Immunol. 2020; 205 (5): 1217-27.
46. Shakiba M, Zumbo P, Espinosa-Carrasco G, Menocal L, Dündar F, Carson S E, et al. TCR signal strength defines distinct mechanisms of T cell dysfunction and cancer evasion. Journal of Experimental Medicine. 2021; 219 (2).
47. Guedan S, Chen X, Madar A, Carpenito C, McGettigan S E, Frigault M J, et al. ICOS-based chimeric antigen receptors program bipolar TH17/TH1 cells. Blood. 2014; 124 (7): 1070-80.
48. Pule M A, Straathof K C, Dotti G, Heslop H E, Rooney C M, and Brenner M K. A chimeric T cell antigen receptor that augments cytokine release and supports clonal expansion of primary human T cells. Molecular Therapy. 2005; 12 (5): 933-41.
49. Song D G, Ye Q, Poussin M, Harms G M, Figini M, and Powell D J, Jr. CD27 costimulation augments the survival and antitumor activity of redirected human T cells in vivo. Blood. 2012; 119 (3): 696-706.
50. Goodman D B, Azimi C S, Kearns K, Talbot A, Garakani K, Garcia J, et al. Pooled screening of CAR T cells identifies diverse immune signaling domains for next-generation immunotherapies. Sci Transl Med. 2022; 14 (670): eabm1463.
51. Gill S, and Brudno J N. CAR T-Cell Therapy in Hematologic Malignancies: Clinical Role, Toxicity, and Unanswered Questions. American Society of Clinical Oncology Educational Book. 2021 (41): e246-e65.
52. Giavridis T, van der Stegen S J C, Eyquem J, Hamieh M, Piersigilli A, and Sadelain M. CAR T cell-induced cytokine release syndrome is mediated by macrophages and abated by IL-1 blockade. Nat Med. 2018; 24 (6): 731-8.
53. Norelli M, Camisa B, Barbiera G, Falcone L, Purevdorj A, Genua M, et al. Monocyte-derived IL-1 and IL-6 are differentially required for cytokine-release syndrome and neurotoxicity due to CAR T cells. Nature Medicine. 2018; 24 (6): 739-48.
54. Finney H M, Lawson A D, Bebbington C R, and Weir A N. Chimeric receptors providing both primary and costimulatory signaling in T cells from a single gene product. J Immunol. 1998; 161 (6): 2791-7.
55. Guedan S, Posey A D, Jr., Shaw C, Wing A, Da T, Patel P R, et al. Enhancing CAR T cell persistence through ICOS and 4-1BB costimulation. JCI Insight. 2018; 3 (1).
56. Pollok K E, Kim Y J, Zhou Z, Hurtado J, Kim K K, Pickard R T, et al. Inducible T cell antigen 4-1BB. Analysis of expression and function. J Immunol. 1993; 150 (3): 771-81.
57. Majzner R G, Theruvath J L, Nellan A, Heitzeneder S, Cui Y, Mount C W, et al. CAR T Cells Targeting B7-H3, a Pan-Cancer Antigen, Demonstrate Potent Preclinical Activity Against Pediatric Solid Tumors and Brain Tumors. Clin Cancer Res. 2019; 25 (8): 2560-74.
58. Nguyen P, Okeke E, Clay M, Haydar D, Justice J, O'Reilly C, et al. Route of 41BB/41BBL Costimulation Determines Effector Function of B7-H3-CAR.CD28ζ T Cells. Mol Ther Oncolytics. 2020; 18:202-14.
59. Luan C, Zhou J, Wang H, Ma X, Long Z, Cheng X, et al. Case Report: Local Cytokine Release Syndrome in an Acute Lymphoblastic Leukemia Patient After Treatment With Chimeric Antigen Receptor T-Cell Therapy: A Possible Model, Literature Review and Perspective. Front Immunol. 2021; 12:707191.
60. Tanyi J L, Stashwick C, Plesa G, Morgan M A, Porter D, Maus M V, et al. Possible Compartmental Cytokine Release Syndrome in a Patient With Recurrent Ovarian Cancer After Treatment With Mesothelin-targeted CAR-T Cells. J Immunother. 2017; 40 (3): 104-7.
61. Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med. 2018; 24 (5): 563-71.
53. Titov A, Kaminskiy Y, Ganeeva I, Zmievskaya E, Valiullina A, Rakhmatullina A, et al. Knowns and Unknowns about CAR-T Cell Dysfunction. Cancers (Basel). 2022; 14 (4).
54. Gumber D, and Wang L D. Improving CAR-T immunotherapy: Overcoming the challenges of T cell exhaustion. eBioMedicine. 2022; 77:103941.
55. Tan J, Jia Y, Zhou M, Fu C, Tuhin I J, Ye J, et al. Chimeric antigen receptors containing the OX40 signalling domain enhance the persistence of T cells even under repeated stimulation with multiple myeloma target cells. Journal of Hematology & Oncology. 2022; 15 (1): 39.
56. Boucher J C, Li G, Kotani H, Cabral M L, Morrissey D, Lee S B, et al. CD28 Costimulatory Domain-Targeted Mutations Enhance Chimeric Antigen Receptor T-cell Function. Cancer Immunol Res. 2021; 9 (1): 62-74.
57. Deng Q, Han G, Puebla-Osorio N, Ma M C J, Strati P, Chasen B, et al. Characteristics of anti-CD19 CAR T cell infusion products associated with efficacy and toxicity in patients with large B cell lymphomas. Nature Medicine. 2020; 26 (12): 1878-87.
58. Hay K A, Hanafi L A, Li D, Gust J, Liles W C, Wurfel M M, et al. Kinetics and biomarkers of severe cytokine release syndrome after CD19 chimeric antigen receptor-modified T-cell therapy. Blood. 2017; 130 (21): 2295-306.
59. Talleur A C, Qudeimat A, Metais J Y, Langfitt D, Mamcarz E, Crawford J C, et al. Preferential expansion of CD8+ CD19-CAR T cells postinfusion and the role of disease burden on outcome in pediatric B-ALL. Blood Adv. 2022; 6 (21): 5737-49.
60. Turtle C J, Hanafi L A, Berger C, Gooley T A, Cherian S, Hudecek M, et al. CD19 CAR-T cells of defined CD4+: CD8+ composition in adult B cell ALL patients. J Clin Invest. 2016; 126 (6): 2123-38.
61. Dobin A, Davis C A, Schlesinger F, Drenkow J, Zaleski C, Jha S, et al. STAR: ultrafast universal RNA-seq aligner. Bioinformatics. 2013; 29 (1): 15-21.
62 Anders S, Pyl P T, and Huber W. HTSeq—a Python framework to work with high-throughput sequencing data. Bioinformatics. 2015; 31 (2): 166-9.
63 Love M I, Huber W, and Anders S. Moderated estimation of fold change and dispersion for RNA-seq data with DESeq2. Genome Biol. 2014; 15 (12): 550.
64. Kanehisa M, Furumichi M, Tanabe M, Sato Y, and Morishima K. KEGG: new perspectives on genomes, pathways, diseases and drugs. Nucleic Acids Res. 2017; 45 (D1): D353-d61.
65. Li B, and Dewey C N. RSEM: accurate transcript quantification from RNA-Seq data with or without a reference genome. BMC Bioinformatics. 2011; 12:323.
67. Ozawa T, and James C D. Establishing intracranial brain tumor xenografts with subsequent analysis of tumor growth and response to therapy using bioluminescence imaging. J Vis Exp. 2010 (41).

1. A chimeric antigen receptor (CAR) comprising:
(a) an extracellular region comprising an antigen binding domain;
(b) a transmembrane region; and
(c) an intracellular region comprising an effector domain and a TMIGD2 costimulatory domain.

2. The CAR of claim 1, wherein the TMIGD2 costimulatory domain comprises the intracellular region of TMIGD2.

3. (canceled)

4. The CAR of claim 2, wherein the TMIGD2 costimulatory domain comprises an amino acid sequence with at least 75% identity to a sequence selected from the group consisting of residues 172-282 of SEQ ID NO:3, 172-278 of SEQ ID NO:4, and residues 52-162 of SEQ ID NO:5.

5. The CAR of claim 1, wherein the antigen binding domain specifically binds a tumor-associated antigen.

6. The CAR of claim 5, wherein the tumor-associated antigen is selected from the group consisting of HHLA2, CD19; CD20; BCMA; CD22; CD3; CEACAM6; c-Met; EGFR; EGFRvIII; ErbB2; ErbB3; ErbB4; EphA2; IGF1R; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTβR; LIFRβ; LRP5; MUC1; OSMRβ; TCRα; TCRβ; CD25; CD28; CD30; CD33; CD52; CD56; CD79a; CD79b; CD80; CD81; CD86; CD123; CD171; CD276; B7-H3; B7H4; TLR7; TLR9; PTCH1; WT-1; HA1-H; Robo1; α-fetoprotein (AFP); Frizzled; OX40; PRAME; and SSX-2 antigen.

7. The CAR of claim 1, wherein the antigen binding domain comprises an scFv.

8. The CAR of claim 7, wherein the antigen binding domain comprises a glycine-serine linker comprising (GlyₓSer_{y})_{z}, wherein x and y are each independently an integer from 0 to 10, provided that x and y are not both 0, and z is an integer from 1 to 10.

9. (canceled)

10. (canceled)

11. The CAR of claim 1, wherein the extracellular region further comprises an N-terminal leader sequence and a hinge region comprising an amino acid sequence with at least 75% identity to the amino acid sequence set forth in SEQ ID NO: 2.

12. (canceled)

13. (canceled)

14. (canceled)

15. The CAR of claim 1, wherein the transmembrane region comprises a CD8α transmembrane region comprising an amino acid sequence with at least 75% identity to the amino acid sequence set forth in SEQ ID NO:1.

16. (canceled)

17. (canceled)

18. The CAR of claim 1, wherein the effector domain is a CD3ζ effector domain comprising an amino acid sequence with at least 75% identity to the amino acid sequence set forth in SEQ ID NO:5.

19. (canceled)

20. (canceled)

21. The CAR of claim 1, wherein the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:1.

22. The CAR of claim 1, wherein the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:6.

23. The CAR of claim 1, wherein the CAR comprises (a) a sequence selected from the group consisting of residues 172-282 of SEQ ID NO:3, 172-278 of SEQ ID NO:4, and residues 52-162 of SEQ ID NO:5; and (b) the sequence set forth in SEQ ID NO:1; and (c) the sequence set forth in SEQ ID NO:6.

24. (canceled)

25. (canceled)

26. A vector comprising a nucleic acid sequence encoding a chimeric antigen receptor (CAR) comprising:
(a) an extracellular region comprising an antigen binding domain;
(b) a transmembrane region; and
(c) an intracellular region comprising an effector domain and a TMIGD2 costimulatory domain.

27. (canceled)

28. (canceled)

29. (canceled)

30. (canceled)

31. (canceled)

32. The vector of claim 26, further comprising a nucleic acid sequence encoding a transduction marker polypeptide and a nucleic acid sequence encoding a self-cleaving peptide, wherein the nucleic acid sequence encoding the self-cleaving peptide is 5′ of the nucleic acid sequence encoding the marker polypeptide.

33. (canceled)

34. (canceled)

35. (canceled)

36. (canceled)

37. An isolated cell expressing the CAR of claim 1.

38. (canceled)

39. (canceled)

40. (canceled)

41. (canceled)

42. (canceled)

43. (canceled)

44. (canceled)

45. A method of treating a disease or condition in a subject in need thereof comprising administering to the subject an effective amount of a cell expressing a chimeric antigen receptor (CAR) comprising:
(a) an extracellular region comprising an antigen binding domain;
(b) a transmembrane region; and
(c) an intracellular region comprising an effector domain and a TMIGD2 costimulatory domain.

46. The method of claim 45, wherein the disease or condition is a malignancy.

47. The method of claim 46, wherein the malignancy is a cancer.

48. The method of claim 47, wherein the cancer is selected from the group consisting of prostate cancer, liver cancer, melanoma, leukemia, lymphoma, breast cancer, ovarian cancer, pancreatic cancer, colorectal cancer, lung cancer, bladder cancer, renal cancer, brain cancer, stomach cancer, small intestine cancer, bone cancer, cervix cancer, endometrium cancer, eye cancer, gallbladder cancer, thyroid cancer, thymus cancer, sarcoma, and osteosarcoma.

49. (canceled)

50. (canceled)

51. (canceled)

52. (canceled)

53. (canceled)
